(19) United States
(12) Patent Application Publication
(10) Pub. No.: US 2014/0100224 A1
(43) Pub. Date: Apr. 10, 2014

(54) NEUROTROPHIN MIMETICS AND USES THEREOF

(71) Applicants: The University of North Carolina at Chapel Hill, Chapel Hill, NC (US); U.S. Government represented by the Department of Veterans Affairs, Washington, DC (US); The Regents of the University of California, Oakland, CA (US)

(72) Inventors: Frank M. Longo, Menlo Park, CA (US); Stephen M. Massa, Burlingame, CA (US)

(73) Assignees: The University of North Carolina at Chapel Hill, Chapel Hill, NC (US); U.S. Government represented by the Department of Veterans Affairs, Washington, DC (US); The Regents of the University of California, Oakland, CA (US)

(21) Appl. No.: 13/927,302

(22) Filed: Jun. 26, 2013

Related U.S. Application Data
(63) Continuation of application No. 12/910,349, filed on Oct. 22, 2010, now abandoned.
Application No. 12/910,349 is a continuation of application No. 12/718,675, filed on Mar. 5, 2010, now abandoned.
Application No. 12/718,675 is a continuation-in-part of application No. 11/396,936, filed on Apr. 3, 2006, now Pat. No. 7,723,328.
(60) Provisional application No. 60/671,785, filed on Apr. 15, 2005.
(60) Provisional application No. 61/158,306, filed on Mar. 6, 2009.
(60) Provisional application No. 61/164,282, filed on Mar. 27, 2009.

(51) Int. Cl. C07D 265/30; C07D 233/61; C07D 473/08; C07D 413/06; C07D 413/12

(52) U.S. Cl. CPC C07D 265/30; C07D 413/06; C07D 413/12; C07D 473/08; C07D 233/61
USPC 514/234.2; 544/168; 544/166; 544/121; 544/139; 544/141; 544/271; 544/118; 548/338.1; 514/237.8; 514/237.5; 514/231.2; 514/235.8; 514/237.2; 514/263.35; 514/263.22; 514/399

(57) ABSTRACT

The present application is related to compounds which are novel neurotrophin mimetics. The application also discloses the treatment of disorders associated with p75 expression, such as degradation or dysfunction of cells expressing p75 in a mammal by administering an effective amount of such compounds.

### RELATED APPLICATIONS

This application is a Continuation of U.S. patent application Ser. No. 12/910,349, filed on Oct. 22, 2010, which is a Continuation of U.S. patent application Ser. No. 12/718,675, filed on Mar. 5, 2010, which is a Continuation-in-Part of U.S. Ser. No. 11/396,936, filed on Apr. 3, 2006, now U.S. Pat. No. 7,723,328, which claims priority to U.S. Ser. No. 60/671,785, filed Apr. 15, 2005, each of which is herein incorporated by reference in its entirety. U.S. Ser. No. 12/718,675 also claims priority to U.S. Ser. No. 61/158,306, filed Mar. 6, 2009 and U.S. Ser. No. 61/164,282, filed Mar. 27, 2009, each of which is herein incorporated by reference in its entirety.

### GOVERNMENT SUPPORT

This invention was made with government support under Grant No. NS30687 awarded by the National Institutes of Health. The Government has certain rights in the invention.

### TECHNICAL FIELD

The present application generally relates to compounds having a binding specificity for p75^{NTR} molecule and to the use of such compounds in the treatment of disorders involving degradation or dysfunction of cells expressing p75, including, for example neurodegenerative disorders.

TABLE OF ABBREVIATIONS

2D—two-dimensional
3D—three-dimensional
Aβ—amyloid-β
Ab—antibody
AD—Alzheimer's disease
BCA—bicinchoninic acid
BDNF—brain-derived neurotrophic factor
b.i.d.—twice daily
cm—centimeter
d—day
D—Dalton
DMEM—Dulbecco's Modified Eagle Media
ECL—electrogenerated chemiluminescence
EDTA—ethylenediamine tetraacetic acid
ELISA—Enzyme Linked ImmunoSorbent Assay
ERK—extracellular signal-regulated protein kinase
FBS—fetal bovine serum
g—gram
h—hour
HBA—hydrogen bond acceptor
HBD—hydrogen bond donor
HEPES—4-2-hydroxyethyl-1-piperazineethanesulfonic acid
HRP—horseradish peroxidase
IgG—Immunoglobin G
IP—Intraperitoneal
IV—intravenous
K³²—lysine residue number 32
kcal—kilocalorie
kg—kilogram
MBP—myelin basic protein
mg—milligram
min—minute
ml—milliliter
mM—millimolar
mol—mole
MTT—3-(4,5-dimethylthiazol-2-yl)-2,5-diphenyltetrazolium bromide
MW—molecular weight
NaCl—sodium chloride
ng—nanogram
nM—nanomolar
NS—not significant
NMR—nuclear magnetic resonance
NGF—nerve growth factor
nM—nanomolar
p—probability
p75^{NTR}—p75 neurotrophin receptor
PBS—phosphate-buffered saline
pmol—picomole
PMSF—phenylmethylsulfonyl fluoride
PO—per os (by mouth)
pro-NGF—unprocessed precursor of NGF
PVDF—Polyvinylidine Difluoride
SDS—sodium dodecyl sulfate
SE—standard error
s.e.m.—standard error of measurement
Tris—2-Amino-2-(hydroxymethyl)-1,3-propanediol
TUNEL—Terminal deoxynucleotidyl transferase-mediated
deoxyuridine triphosphate nick-end labeling
μg—microgram
μl—microliter
μM—micromolar
%—percent
° C.—degrees Celsius
≧—greater than or equal to
>—greater than
≦—less than or equal to
<—less than

### BACKGROUND

Neurotrophins are polypeptides that play a role in the development, function, and/or survival of certain cells, including neurons, oligodendrocytes, Schwann cells, hair follicle cells, and other cells. The death or dysfunction of neurons and other cell types has been directly implicated in a number of neurodegenerative disorders. It has been suggested that alterations in neurotrophin localization, expression levels of neurotrophins, and/or expression levels of the receptors that bind neurotrophins are therefore linked to neuronal degeneration. Degeneration occurs in the neurodegenerative disorders Alzheimer's, Parkinson's and ALS, among others. Degeneration of oligodendrocytes can occur in central nervous system injury, multiple sclerosis, and other pathological states.

A variety of neurotrophins have been identified, including Nerve Growth Factor (NGF), Neurotrophin-3 (NT-3), Neurotrophin-4/5 (NT-4/5), Neurotrophin 6 (NT-6) and Brain Derived Neurotrophic Factor (BDNF). Neurotrophins are found in both precursor form, known as pro-neurotrophins, and in mature form. The mature forms are proteins of about 120 amino acids in length that exist in physiological states as stable, non-covalent approximately 25 kDa homodimers. Each neurotrophin monomer includes three solvent-exposed β-hairpin loops, referred to as loops 1, 2, and 4 that exhibit relatively high degrees of amino acid conservation across the neurotrophin family.

Mature neurotrophins bind preferentially to the receptors Trk and p75^{NTR} (p75 neurotrophin receptor, also called the Low Affinity Nerve Growth Factor Receptor or LNGFR) while pro-neurotrophins, which contain an N-terminal domain proteolytically removed in mature forms, interact principally with p75^{NTR} and through their N-terminal domains, with the sorting receptor sortilin (Fahnestock, M., et al. (2001) Mol Cell Neurosci 18, 210-220; Harrington, A. W. et al. (2004) Proc Natl Acad Sci USA 101, 6226-6230; Nykiaer. A. et al., (2004) Nature 427, 843-848). p75^{NTR} interacts with Trks and modulates Trk signaling, but is also independently coupled to several signaling systems, including pro-survival signals, IRAK/TRAF6/NF.kappa.B, PI3/AKT, and pro-apoptotic signals, NRAGE/JNK (Mamidipudi, V., et al. (2002) J Biol Chem 277, 28010-28018; Roux, P. P., et al. (2001) J Biol Chem 276, 23097-23104; Salehi, A. H., et al. (2000) Neuron 27, 279-288).

When administered for therapeutic use, neurotrophins exhibit suboptimal pharmacological properties, including poor stability with low serum half lives, likely poor oral bioavailability, and restricted central nervous system penetration (Podulso, J. F., Curran, G. L. (1996) Brain Res Mol Brain Res 36, 280-286; Saltzman, W. M., et al (1999) Pharm Res 16, 232-240; Partridge, W. M. (2002) Adv Exp Med Bio 513, 397-430). Additionally, the highly pleiotropic effects of neurotrophins achieved through action of the dual receptor signaling network increases the chances of adverse effects.

It has been suggested that the unliganded form of p75^{NTR} is proapoptotic, and that homodimerization induced by neurotrophin binding eliminates the effect (Wang, J. J., et al (2000) J Neurosci Res 60, 587-593), consistent with studies showing no effects on survival of monomeric p75^{NTR} ligands, including monovalent Fabs (Maliartchouk, S., et al (2000) J Biol Chem 275, 9946-9956) and monomeric cyclic peptides (Longo, F. M., (1997) J Neurosci Res 48, 1-17), while related bivalent forms in each study promote cell survival. However, these monomeric ligands may not engage the receptor in the same way as the natural ligands. Though active NGF is a homodimers containing 2 potential p75^{NTR} binding sites, recent structural evidence suggests that it engages only one p75^{NTR} molecule, disallowing the binding of another (He, X. L., (2004) Science 304, 870-875).

Unfortunately, technical and ethical considerations have thus far hampered the development of therapeutic agents based upon neurotrophins. For example, it is technically difficult to produce sufficient quantities of pure neurotrophins using recombinant DNA techniques. Additionally, although it is possible to utilize human fetal cells to produce neurotrophins, the ethical ramifications raised by the use of such cells (typically obtained from an aborted fetus) have all but prevented the utilization of this approach. Accordingly, there is an unmet need in the art for the development of small molecule agents with favorable drug-like features based upon neurotrophins that are capable of targeting specific neurotrophin receptors for use in the treatment of disorders or diseases.

### SUMMARY

This application generally discloses compounds having binding specificity for p75^{NTR}, as well as to methods for the preparation and use of such compounds, and to pharmaceutical compositions containing the same. More specifically, compounds of the present application are represented by the general structures:

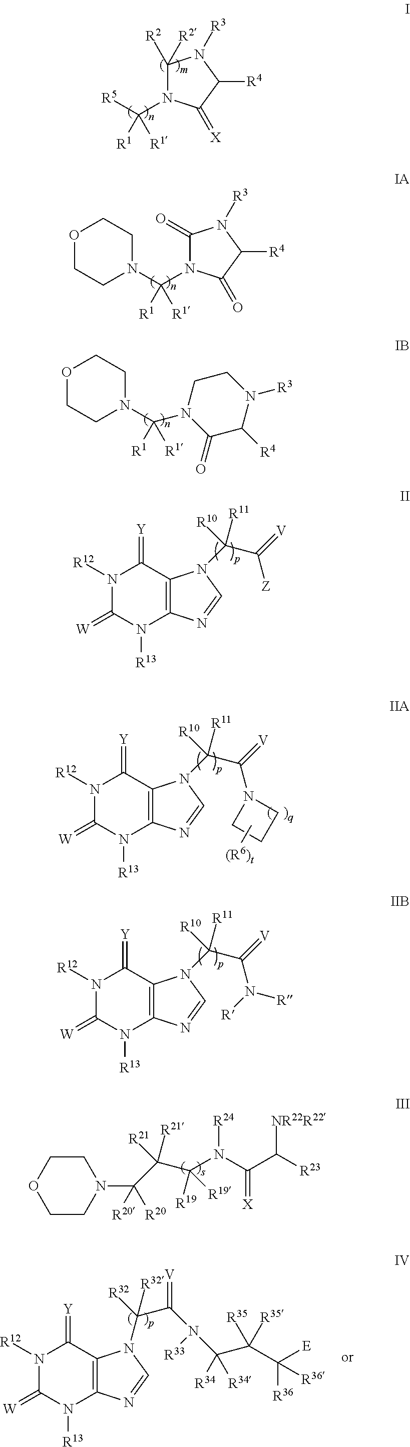

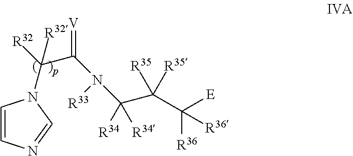

including pharmaceutically acceptable salts, esters, solvates, and prodrugs thereof, wherein R¹, R^{1′}, R², R^{2′}, R³, R⁴, R⁵, R⁶, R¹⁰, R¹¹, R¹², R¹³, R¹⁹, R^{19′}, R²⁰, R^{20′}, R²¹, R^{21′}, R²², R²³, R²⁴, R³⁰, R³¹, R³², R^{32′}, R³³, R³⁴, R^{34′}, R³⁵, R^{35′}, R³⁶, R^{36′}, E, V, W, X, Y, Z m, n, p, q, r, s, t, are as defined below.

Additionally disclosed are stereoisomers of having the structural formula:

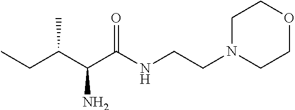 [SMILES: CC[C@H](C)[C@H](N)C(=O)NCCN1CCOCC1]

### (2S,3S)-2-amino-3-methyl-N-(2-morpholinoethyl)pentanamide

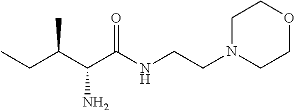 [SMILES: CC[C@@H](C)[C@@H](N)C(=O)NCCN1CCOCC1]

### (2R,3R)-2-amino-3-methyl-N-(2-morpholinoethyl)pentanamide

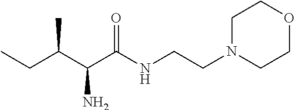 [SMILES: CC[C@@H](C)[C@H](N)C(=O)NCCN1CCOCC1]

### (2S,3R)-2-amino-3-methyl-N-(2-morpholinoethyl)pentanamide

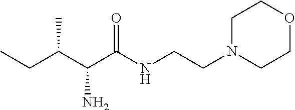 [SMILES: CC[C@H](C)[C@@H](N)C(=O)NCCN1CCOCC1]

### (2R,3S)-2-amino-3-methyl-N-(2-morpholinoethyl)pentanamide

Generally disclosed herein are methods of treating a neurodegenerative or other disorder in a subject, comprising administering to the subject an effective amount of a compound having binding specificity for a p75^{NTR}. Also disclosed herein are methods of facilitating neural, oligodendrocyte, or other cell survival comprising treating such cells with a compound having binding specificity for a p75^{NTR} molecule. Further disclosed herein are methods for treating a disorder associated with p75 expression.

One object of the presently disclosed subject matter having been stated hereinabove, and which is addressed in whole or in part by the present presently disclosed subject matter, other objects will become evident as the description proceeds when taken in connection with the accompanying examples and drawings as best described hereinbelow.

### BRIEF DESCRIPTION OF THE DRAWINGS

FIG. 1a is a ribbon representation of the X-ray crystal structure of human NGF with β-turn loops 1, 2, and 4 designated. The average side chain positions for loop 1 are illustrated.

FIG. 1b represents the comparison of peptide sequences (SEQ ID NOs:1-3) of loop 1 from NGF and NT3 from the indicated species and the assignment of pharmacophores. Positively ionizable groups are signified by “+”. “HBD” and “HBA” represent hydrogen bond donor and hydrogen bond acceptor, respectively.

FIG. 1c shows application of the pharmacophoric features to a 3D loop model. Hydrogen bonding features are represented by pairs of spheres with their relative positions indicating the locations of the acceptor and the donor. One of the spheres of the pair is centered on putative acceptor/donor features in the model, while the other indicates the target location of a complementary feature on any potentially interacting molecule. The diameter of the spheres represents the spatial tolerance for chemical feature matching in 3D conformer library scans.

FIG. 1d is a 3D loop model disclosing representative fits to the pharmacophore of two compounds identified by application of the novel pharmacophore in library screening subsequently found to be active as disclosed herein.

FIG. 2a is a series of fluorescence photomicrographs of E16-17 mouse hippocampal neuronal cultures treated with culture medium only (CM) or medium containing BDNF or Compound (i) (referred to in the figures as “LM11A-28” or “28”), Compound (II) (referred to in the figures as “LM11A-7” or “7”), Compound (iii) (referred to in the figures as “LM11A-24” or “24”), Compound (iv) (referred to in the figures as “LM11A-31” or “31”), or Compound (v) (referred to in the figures as “LM11A-36” or “36”). The cultures were stained for expression of the neuron-specific, growth-associated protein GAP43 at 48 hours post treatment. The 2D structure of each compound is located adjacent to each image.

FIG. 2b is a series of neuron survival dose-response curves of BDNF, NGF, and Compounds (i-v), showing similar potency and maximal responses between NGF and Compounds (i-iv) up to 5 nM, with no response to Compound (v). BDNF has similar potency, but a higher maximal response. Each of Compounds (i-v), show a decrementing response above 5 nM. Survival was determined as the total number of cells in each well that were both morphologically intact and filled with blue formazan MTT-conversion product (Longo, F. M., Manthorpe, M., Xie, Y. M., and Varon, S. (1997) J Neurosci Res 48, 1-17). Counts were normalized to survival achieved with 25 ng/ml BDNF or to baseline survival. n is 4-18 for all determinations. Symbols and bars indicate mean+/−s.e.m., and lines are fits of a single exponential rise model to the data. Dotted lines in each graph represent the fitted NGF response.

FIG. 3a is a series of NGF/p75^{NTR}-Fc binding curves, in the presence of increasing concentrations of Compound (iv), as detected by NGF ELISA. Symbols are mean+/−s.e.m. n>10 for all determinations. Lines represent fitting to a modified Gaddum/Schild equation, with an overall R² value of 0.93 for Compound (iv). Also, P<0.0001 by ANOVA with post-hoc Bonferroni/Dunn testing, for comparisons between binding curves at 0 nM compound and curves with ≧500 nM Compound (iv). K_{D} for NGF in the absence of compounds was 0.8-0.9 nM, consistent with previous reports of approximately 1 nM (Nykjaer, A. et al., (2004) Nature 427, 843-848). The symbols “”, “ [symbol] ”, “▪”, “⋄”, “▴”, and “◯” represent Compound (iv) concentrations of zero, 125, 500, 1,500, 4,500, and 10,000 nanomolar, respectively.

FIG. 3b is a series of NGF/p75^{NTR}-Fc binding curves, in the presence of increasing concentrations of Compound (iii), as detected by NGF ELISA. Symbols are mean+/−s.e.m. n>10 for all determinations. Lines represent fitting to a modified Gaddum/Schild equation, with an overall R² value of 0.96 for Compound (iii). Also, P<0.0001 by ANOVA with post-hoc Bonferroni/Dunn testing, for comparisons between binding curves at 0 nM compound and curves with ≧125 nM Compound (iii). K_{D} for NGF in the absence of compounds was 0.8-0.9 nM, consistent with previous reports of approximately 1 nM (Nykjaer, A. et al., (2004) Nature 427, 843-848). The symbols “”, “ [symbol] ”, “▪”, “⋄”, “▴”, and “◯” represent Compound (iii) concentrations of zero, 125, 500, 1,500, 4,500, and 10,000 nanomolar, respectively.

FIG. 3c is a series of NGF/TrkA-Fc binding curves in the presence of increasing concentrations of Compound (v), showing no significant effect up to 10,000 nM. Symbols are mean+/−s.e.m. n≧10 for all determinations. The symbols “”, “ [symbol] ”, and “▪”, represent Compound (v) concentrations of zero, 4,500, and 10,000 nanomolar, respectively.

FIG. 3d is a series of NGF/TrkA-Fc binding curves in the presence of increasing concentrations of Compound (iv) showing no compound effects up to 10,000 nM. Symbols are mean+/−s.e.m. n≧4 for all determinations. The symbols “”, “ [symbol] ”, and “▪”, represent Compound (iv) concentrations of zero, 4,500, and 10,000 nanomolar, respectively.

FIG. 3e is a series of NGF/TrkA-Fc binding curves in the presence of increasing concentrations of Compound (iii) showing no compound effects up to 10,000 nM. Symbols are mean+/−s.e.m. n≧4 for all determinations. The symbols “”, “ [symbol] ”, and “▪”, represent Compound (iii) concentrations of zero, 4,500, and 10,000 nanomolar, respectively.

FIG. 3f is a digital image of a western blot showing displacement of anti-p75^{NTR} Ab 9651 from anti-p75^{NTR}-expressing 3T3 cells by Compound (iv), but not Compound (v). The upper panel represents IgG heavy chain, the lower panel represents β-actin. The graph represents quantitation. The bars represent mean+/−s.e.m., normalized to bound antibody (lane 4). n=4 for each condition. A single asterisk (*) represents P<0.0005, for comparison with binding in the absence of compound, by Student t-test. Antibody and compound treatments are designated above each lane. Ab 9651 did not bind to p75^{NTR}-negative cells (lanes 1 and 2). Ab 9651 bound to p75^{NTR}-positive cells (lane 4) and was significantly displaced by Compound (iv) (lane 5), while Compound (v) had no effect (lane 6).

FIG. 3g is a bar graph showing that Ab 9651 has no effect on baseline survival (CM), partially inhibits BDNF, and completely inhibits Compound (iii) and Compound (iv) promotion of hippocampal neuron survival. The solid bars represent non-immune serum treatment. The shaded bars represent Ab 9651 treatment. The bars represent mean+/−s.e.m. n≧26 for each condition. Double asterisks (**) represent P<0.00001 (for comparisons between Ab 9651 and non-immune). NS represents not significant by Student t-test. Survival in the presence of BDNF+Ab 9651 is shown to be significantly greater than CM+Ab 9651 (P<0.00001), while the differences between CM and Compound (iii), Compound (iv), and Compound (v) in the presence of antibody are not significant.

FIG. 3h is a bar graph showing that p75^{NTR}-deficiency partially inhibits BDNF and completely inhibits NGF, Compound (iii), and Compound (iv) promotion of hippocampal neuron survival. Neurotrophins were applied at 1.8 nM, and compounds at 5 nM. The solid bars represent p75^{NTR+/+} cells. The shaded bars represent cells. The bars represent mean+/−s.e.m. n≧5 for each condition. The single asterisk (*) represents P<0.05, the double asterisk (**) represents P<0.005. NS represents not significant (for comparisons between knockout and wild type) by Student t-test. In p75^{NTR-/-} cultures, BDNF treatment produced greater survival than NGF (P<0.05) or Compounds (iii-v) (P<0.01). There was no significant difference in baseline survival between the genotypes.

FIG. 3i shows digital images of western blots of hippocampal neuron cultures using anti-phosphorylated Trk^{Y490}, compared with total TrkB. BDNF activated TrkB, while NGF and Compounds (iii-v) resulted in no detectable activation at 10 or 30 minutes.

FIG. 3j shows digital images of western blots of TrkA-expressing 3T3 cells using anti-phosphorylated Trk^{Y490} compared with total TrkA. NGF is shown to activate TrkA, while Compound (iv) produced no detectable activation. Results of two additional independent assays for TrkB and TrkA activation were identical.

FIGS. 4a-4d are digital images of western blots of extracts of hippocampal cultures treated with culture media (C), BDNF (B) at 50 ng/ml, NGF (N) at 50 ng/ml, or Compounds (iii-v) at 20 nM showing representative bands corresponding to phosphorylated signaling factors (open arrowheads) and the corresponding total factor (filled arrowheads) and quantitation of the ratio of phospho- to total factor, indicating degree of activation. Bars indicate mean+/−s.e.m. Solid bars represent sampling at 10 minutes. Shaded bars represent sampling at 30 minutes. n=6 independent blots for each determination. Single asterisks (*) represent P<0.001 for comparison with CM by Student t-test. Other comparisons are as indicated, with P values by Student t-test indicated above each bracket.

FIG. 4a is a digital image of a western blot indicating NFκB-p65 activation analysis, showing similar activation kinetics for all biologically active treatments.

FIG. 4b is a digital image of a western blot representing AKT activation analysis, showing a small lag in activation by the active compounds relative to NGF.

FIG. 4c is a digital image of a western blot representing ERK44 activation analysis, showing less activation at 10 minutes for the compounds relative to NGF.

FIG. 4d is the digital image of a western blot representing ERK42 activation analysis, showing prolonged activation with BDNF treatment relative to NGF and Compounds (iii-v).

FIG. 4e is a bar graph indicating survival of hippocampal neurons in cultures treated with signaling pathway inhibitors and BDNF (25 ng/ml), NGF (25 ng/ml), or Compounds (iii-v) (5 nM), showing substantial inhibition by NFκB and P13K pathway inhibitors, small effects of ERK inhibition on BDNF and NGF activity, and no effect of ERK inhibition on the activity of Compounds (iii-v). SN50 is an NFκB translocation inhibitor. LY represents LY294002, a P13K inhibitor. PD represents PD98059, an ERK inhibitor. n=18 for each bar, showing mean±s.e.m. NS represents that the data is not significant. A single asterisk (*) indicates P<0.05, double asterisks (**) indicates that P<0.001 for comparison with control (no inhibitor) in each group. The open, solid, lighter-shaded, and darker-shaded bars represent control, SN50, LY, and PD, respectively.

FIG. 4f is the digital image of a western blot of signaling activation analysis of NFκB pathway activation. Bars indicate mean+/−s.e.m. n≧6 for each condition. P values are as indicated. Activation is detected between 0 and 0.5 nM for NFκB, reaching a plateau level at 5 nM.

FIG. 4g is the digital image of a western blot of signaling activation analysis of AKT pathway activation. Bars indicate mean+/−s.e.m. n≧6 for each condition. P values are as indicated. Activation is detected between 0.5 and 1 nM for AKT, reaching a plateau level at 5 nM.

FIG. 4h is the digital image of a western blot indicating AKT activation by growth factors and compounds in p75^{NTR-/-} cells. n≧9 for each condition. There are no significant differences between culture medium alone and NGF or Compounds (iii-v).

FIG. 5a is a bar graph disclosing that Compounds (iii-v) do not promote death of mature oligondendrocytes and inhibits proNGF-induced death. Mature oligondendrocytes were treated as indicated and cell death assessed by determining the proportion of MBP-positive cells that are also TUNEL-positive. In the absence of pro-NGF, compounds did not promote cell death. In the presence of 2.8 ng/ml (0.05 nM) proNGF, Compound (iii) and Compound (iv), but not Compound (v), blocked cell death. Bars represent mean±s.e.m. n≧2 for each condition except for 1 nM Compound (v) with proNGF which had a single determination. P<0.05, by Student t-test for comparisons with proNGF treatment without compounds. The closed, lighter-shaded, and darker-shaded bars represent Compound (iii), Compound (iv), and Compound (v), respectively.

FIG. 5b is a line graph showing proNGF displacement from p75^{NTR} by Compound (iii) and Compound (iv). 100 ng/ml proNGF was incubated with the indicated concentrations of compounds and detected by ELISA. n=4 for each condition. Symbols indicate means+/−s.e.m. The signal from all compound-treated samples were significantly less than proNGF alone, with P<0.01 by Student t-test. The symbols “Δ” and “” represent Compound (v) and Compound (iii), respectively.

FIGS. 6a-6b demonstrate that Compound (iii) blocks Aβ-induced neural degeneration. FIG. 6a is a bar graph representing percentage of surviving hippocampal neuronal cells after addition of Aβ₁₋₄₂ (10 μM or 30 μM). Addition of Aβ₁₋₄₂ resulted in an approximate 40% loss of neurons after 3 days of exposure. Addition of NGF (100 pg/ml) did not protect against Aβ₁₋₄₂. FIG. 6b is a bar graph representing the percentage of surviving hippocampal neural cells after addition of Aβ₁₋₄₂ (10 μM) with test compounds.

### DETAILED DESCRIPTION

In subjects with disorders related to degeneration or dysfunction of cells expressing p75, such as neurodegenerative disorders, alterations in neurotrophin localization, expression levels of neurotrophins, expression levels of the receptors that bind neurotrophins, and/or receptor signaling and functional outcomes can occur. In addition within these disorders, alterations in signaling pathways or other mechanisms that are linked to p75 receptor mechanisms and that can be regulated by p75 signaling can occur. Other disorders involve cells not expressing the p75 receptor however, cells expressing p75 have the ability to compensate for impairment or loss of non p75-expressing cells. Accordingly, by providing subjects suffering from such disorders with a corresponding neurotrophic factor or mimetic thereof that modulates p75^{NTR} function or proNGF/NGF binding to prevent cellular degeneration or dysfunction, such neural degeneration can be alleviated or prevented. As disclosed herein, methods of treating neurodegenerative and other disorders and/or facilitating cell survival by administering a compound having binding specificity for a p75^{NTR} molecule are provided.

The methods and compounds of the present application relate to compounds having binding specificity for a p75^{NTR} molecule. Compounds having binding specificity for p75^{NTR} are suitable for positively regulating survival and/or inhibiting degeneration of neural and other cells, e.g. inhibition or reversal of neuronal spine loss. Particularly, in cells showing trophic responses to neurotrophins or cells expressing p75^{NTR} either constitutively or in response to injury or disease, the compounds promote survival signaling and/or inhibit degenerative or dysfunctional signaling. In cells susceptible to neurotrophin-induced death, the compounds do not induce apoptosis, but inhibit neurotrophin-mediated death. Such mechanisms are relevant beyond the nervous system and include neurotrophin regulation of p75 receptor-expressing hair follicle cell survival and hair loss.

Additional embodiments and advantages of the application will be set forth in part in the description that follows, and in part will be obvious from the description, or may be learned by practice of the invention. It is to be understood that both the foregoing general description and the following detailed description are exemplary and explanatory only and are not restrictive of the invention as claimed.

### DEFINITIONS

It is to be understood that the terminology used herein is for the purpose of describing particular embodiments only and is not intended to be limiting.

Unless defined otherwise, all technical and scientific terms used herein have the same meaning as commonly understood to one of ordinary skill in the art to which the present application belongs. Although any methods and materials similar or equivalent to those described herein can be used in the practice or testing of the present application, representative methods and materials are herein described.

Following long-standing patent law convention, the terms “a”, “an”, and “the” refer to “one or more” when used in this application, including the claims. Thus, for example, reference to “a carrier” includes mixtures of one or more carriers, two or more carriers, and the like.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the present application. Generally the term “about”, as used herein when referring to a measurable value such as an amount of weight, time, dose, etc. is meant to encompass in one example variations of ±20% or ±10%, in another example ±5%, in another example ±1%, and in yet another example ±0.1% from the specified amount, as such variations are appropriate to perform the disclosed method.

As used herein, the phrase “a disorder involving degeneration or dysfunction of cells expressing p75” includes, but is not limited to disorders related to upregulation of p75. Such disorders include neurodegenerative disorders, as well as conditions involving degeneration of p75^{NTR}-expressing cells, such as hair loss. Within the nervous system, the p75 receptor is expressed by various cell types including neurons, oligodendrocytes, astrocytes and microglia. Compounds targeting p75 receptors expressed by neurons can be used to prevent loss of function, degeneration and/or death of neurons in a number of nervous system disorders including, but not limited to, Alzheimer's disease, Parkinson's disease, Huntington's disease, stroke, traumatic brain injury, spinal cord injury, epilepsy, multiple sclerosis, amyotrophic lateral sclerosis, neuropathies, myopathies and various forms of retinal degeneration. In each of these disorders, neurons and other cells expressing p75 are affected.

Compounds targeting p75 receptors expressed by oligodendrocytes can be used to prevent loss of function, degeneration and/or death of oligodendrocytes in a number of nervous system disorders including, but not limited to, multiple sclerosis, spinal cord injury and perinatal anoxia. Compound targeting p75 receptors expressed by microglia can be used to inhibit deleterious activation of microglia and thereby decrease the inflammatory component of neurodegenerative and other disorders.

Outside of the nervous system, a number of cell populations express the p75 receptor. These include hair follicle cells, hepatic cells, vascular endothelial, vascular smooth muscle cells, cardiomyocytes. In addition, the p75 receptor is expressed by certain tumor cells such as those involved in breast or prostate cancer. Given this expression pattern, compounds targeting p75 receptors can be used for the following indications: to prevent loss of hair follicle cells and thereby prevent hair loss; to prevent hepatic cirrhosis and promote liver regeneration; to regulate angiogenesis and promote neovascularization in the setting of diabetic wounds or other ischemic settings; to prevent cardiomyopathy by preventing myocardial cell loss or by stimulating growth of new cardiomyocytes either in the setting of ischemia or after myocardial infarction; and to inhibit tumor cell growth. In addition p75 is expressed by stem cells and is known to regulate stem cell growth; therefore, p75 ligands can be used to promote stem cell growth as part of a strategy to promote tissue and organ regeneration. P75 receptor ligands can also be used to tag or identify cells expressing p75 or having upregulated p75 as part of diagnostic or cell-harvesting strategy.

As used herein, the teen “neurodegenerative disorder” includes any disorder characterized by neural damage or dysfunction and includes but is not limited to Alzheimer's disease, Huntington's disease, Pick's disease, amyotrophic lateral sclerosis, epilepsy, Parkinson's disease, spinal cord injury, stroke, hypoxia, ischemia, brain injury, diabetic neuropathy, peripheral neuropathy, nerve transplantation, multiple sclerosis, and peripheral nerve injury.

The compounds disclosed herein function as ligands at the p75 neurotrophin receptor and thereby induce intracellular signaling that prevents cellular degeneration or death and/or upregulates cell function or growth. The intracellular signaling mechanisms regulated by the p75 receptor are fundamental mechanisms present in essentially all cell types; therefore, it is expected that any cell or tissue expressing this receptor would be amendable to treatment with these compounds for the goal of preventing cellular or tissue degeneration, promoting cell survival and/or for upregulating function or growth.

The term “alkyl,” alone or in combination, refers to an optionally substituted straight-chain or branched-chain alkyl radical having from 1 to about 20 carbon atoms. The term also includes optionally substituted straight-chain or branched-chain alkyl radicals having from 1 to about 6 carbon atoms as well as those having from 1 to about 4 carbon atoms. Examples of alkyl radicals include methyl, ethyl, n-propyl, isopropyl, n-butyl, isobutyl, sec-butyl, tert-butyl, tert-amyl, pentyl, hexyl, heptyl, octyl and the like. “Branched” refers to an alkyl group in which a lower alkyl group, such as methyl, ethyl or propyl, is attached to a linear alkyl chain “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls. Alkyl groups can be optionally substituted.

The term “heteroalkyl” refers to alkyl groups, as described above, in which one or more skeletal atoms are oxygen, nitrogen, sulfur or combinations thereof. The term heteroalkyl also includes alkyl groups in which one 1 to about 6 skeletal atoms are oxygen, nitrogen, sulfur or combinations thereof, as well as those in which 1 to 4 skeletal atoms are oxygen, nitrogen, sulfur or combinations thereof and those in which 1 to 2 skeletal atoms are oxygen, nitrogen, sulfur or combinations thereof. Heteroalkyl groups are optionally substituted.

The term “alkenyl,” alone or in combination, refers to an optionally substituted straight-chain or branched-chain hydrocarbon radical having one or more carbon-carbon double-bonds and having from 2 to about 18 carbon atoms. The term also includes optionally substituted straight-chain or branched-chain hydrocarbon radicals having one or more carbon-carbon double bonds and having from 2 to about 6 carbon atoms as well as those having from 2 to about 4 carbon atoms. Examples of alkenyl radicals include ethenyl, propenyl, butenyl, 1,4-butadienyl and the like. Suitable alkenyl groups include allyl. The terms “allylic group” or “allyl” refer to the group —CH₂HC═CH₂ and derivatives thereof formed by substitution. Thus, the terms alkenyl and/or substituted alkenyl include allyl groups, such as but not limited to, allyl, methylallyl, di-methylallyl, and the like. The term “allylic position” or “allylic site” refers to the saturated carbon atom of an allylic group. Thus, a group, such as a hydroxyl group or other substituent group, attached at an allylic site can be referred to as “allylic.” “1-alkenyl” refers to alkenyl groups where the double bond is between the first and second carbon atom.

The term “alkynyl,” alone or in combination, refers to an optionally substituted straight-chain or branched-chain hydrocarbon radical having one or more carbon-carbon triple-bonds and having from 2 to about 12 carbon atoms. The term also includes optionally substituted straight-chain or branched-chain hydrocarbon radicals having one or more carbon-carbon triple bonds and having from 2 to about 6 carbon atoms as well as those having from 2 to about 4 carbon atoms. Examples of alkynyl radicals include ethynyl, propynyl, butynyl and the like. “1-alkynyl” refers to alkynyl groups where the triple bond is between the first and second carbon atom.

“Cyclic alkyl” and “cycloalkyl” refer to a non-aromatic mono- or multicyclic ring system of about 3 to about 10 carbon atoms, e.g., 3, 4, 5, 6, 7, 8, 9, or 10 carbon atoms, alternately from about 3 to about 6 carbon atoms. The cycloalkyl group can be optionally partially unsaturated. The cycloalkyl group also can be optionally substituted as defined herein. Representative monocyclic cycloalkyl rings include, but are not limited to, cyclopropyl, cyclobutyl, cyclopentyl, cyclohexyl, cycloheptyl, and the like. Further, the cycloalkyl group can be optionally substituted with a linking group, such as an alkylene group as defined hereinabove, for example, methylene, ethylene, propylene, and the like. In such cases, the cycloalkyl group can be referred to as, for example, cyclopropylmethyl, cyclobutylmethyl, and the like. Additionally, multicyclic cycloalkyl rings include adamantyl, octahydronaphthyl, decalin, camphor, camphane, and noradamantyl.

The term “heterocyclic alkyl” and “heterocycloalkyl” refer to cyclic groups of 3 to 6 atoms, or 3 to 10 atoms, containing at least one heteroatom. In one aspect, these groups contain 1 to 3 heteroatoms. Suitable heteroatoms include oxygen, sulfur, and nitrogen. Heterocyclic groups may be attached through a nitrogen or through a carbon atom in the ring. Suitable heterocyclic groups include pyrrolidinyl, morpholino, morpholinoethyl, and pyridyl. Such groups may be substituted.

The term “aryl” refers to aromatic groups which have 5-14 ring atoms and at least one ring having a conjugated pi electron system and includes carbocyclic aryl, heterocyclic aryl and biaryl groups, all of which may be optionally substituted. The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The aromatic ring(s) can comprise phenyl, naphthyl, biphenyl, diphenylether, diphenylamine and benzophenone, among others. all of which can be optionally substituted. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings. Examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like, all optionally substituted.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

A structure represented generally by a formula such as:

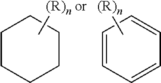

as used herein refers to a 6-carbon ring structure comprising a substituent R group, wherein the R group can be present or absent, and when present, one or more R groups can each be substituted on one or more available carbon atoms of the ring structure. The presence or absence of the R group and number of R groups is determined by the value of the integer n. Each R group, if more than one, is substituted on an available carbon of the ring structure rather than on another R group. For example, the structure:

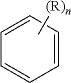

wherein n is an integer from 0 to 2 comprises compound groups including, but not limited to:

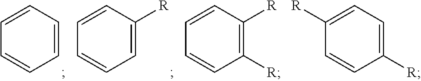

and the like.

The structure:

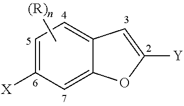

wherein n is one (1) comprises compound groups including:

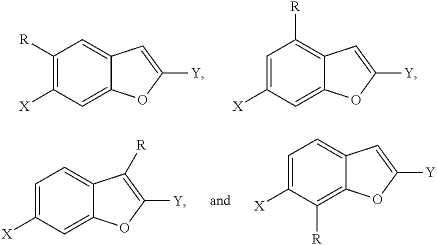

wherein the one (1) R substituent can be attached at any carbon on the benzofuran parent structure not occupied by another designated substituent, as in this case carbon 6 is substituted by X and carbon 2 is substituted by Y.

A dashed line representing a bond in a cyclic ring structure indicates that the bond can be either present or absent in the ring. That is a dashed line representing a bond in a cyclic ring structure indicates that the ring structure is selected from the group consisting of a saturated ring structure, a partially saturated ring structure, and an unsaturated ring structure.

“Carbocyclic aryl” groups are groups wherein the ring atoms on the aromatic ring are carbon atoms. Carbocyclic aryl groups include monocyclic carbocyclic aryl groups and polycyclic or fused compounds such as optionally substituted naphthyl groups.

“Heterocyclic aryl” or “heteroaryl” groups are groups having from 1 to 4 heteroatoms as ring atoms in the aromatic ring and the remainder of the ring atoms being carbon atoms. Suitable heteroatoms include oxygen, sulfur, nitrogen, and selenium. Suitable heteroaryl groups include furanyl, thienyl, pyridyl, pyrrolyl, N-lower alkyl pyrrolyl, pyridyl-N-oxide, pyrimidyl, pyrazinyl, imidazolyl, and the like, all optionally substituted.

The phrase “carbocyclic ring” refers to a saturated or unsaturated monocyclic or bicyclic ring in which all atoms of all rings are carbon. Thus, the term includes cycloalkyl and carbocyclic aryl rings.

The phrase “heterocyclic ring” refers to a saturated or unsaturated monocyclic or bicyclic ring having from 1 to 4 heteroatoms as ring atoms in the aromatic ring and the remainder of the ring atoms being carbon atoms. Thus, the term includes heterocycloalkyl and heterocyclic aryl rings.

The term “optionally substituted” or “substituted” includes groups substituted by one to four substituents, independently selected from lower alkyl, lower aryl, lower aralkyl, lower alicyclic, heterocyclic alkyl, hydroxyl, lower alkoxy, lower aryloxy, perhaloalkoxy, aralkoxy, heteroaryl, heteroaryloxy, heteroarylalkyl, heteroaralkoxy, azido, amino, guanidino, amidino, halo, lower alkylthio, oxo, acylalkyl, carboxy esters, carboxyl, -carboxamido, nitro, acyloxy, aminoalkyl, alkylaminoaryl, alkylaryl, alkylaminoalkyl, alkoxyaryl, arylamino, aralkylamino, phosphono, sulfonyl, -carboxamidoalkylaryl, -carboxamidoaryl, hydroxyalkyl, haloalkyl, alkylaminoalkylcarboxy-, aminocarboxamidoalkyl-, cyano, lower alkoxyalkyl, lower perhaloalkyl, and arylalkyloxyalkyl.

In some embodiments, the compounds described by the presently disclosed subject matter contain a linking group. As used herein, the term “linking group” comprises a chemical moiety which is bonded to two or more other chemical moieties to form a stable structure. Representative linking groups include but are not limited to a furanyl, phenylene, thienyl, or pyrrolyl radical bonded two or more aryl groups.

When a named atom of a ring or chain is defined as being “absent,” the named atom is replaced by a direct bond or is incorporated into double bond along with the atom to which it is attached. When the linking group or spacer group is defined as being absent, the linking group or spacer group is replaced by a direct bond.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

The term “alkenylene” denotes an acyclic carbon chain (i.e., having an open-chain structure) having a carbon-to-carbon double bond and is represented by the formula CₙH₂ₙ₋₂, which optionally can be substituted one or more times. Representative alkenylene groups include, but are not limited to, ethenylene, propenylene, 1- or 2-butenylene, 1-, or 2-pentylene, and the like.

As used herein, the term “acyl” refers to an organic acid group wherein the —OH of the carboxyl group has been replaced with another substituent (i.e., as represented by RCO—, wherein R is an alkyl or an aryl group as defined herein). As such, the term “acyl” specifically includes arylacyl groups, such as an acetylfuran and a phenacyl group. Specific examples of acyl groups include acetyl and benzoyl.

“Alkoxyl” or “alkoxyalkyl” refer to an alkyl-O— group wherein alkyl is as previously described. The term “alkoxyl” as used herein can refer to C₁₋₂₀ inclusive, linear, branched, or cyclic, saturated or unsaturated oxo-hydrocarbon chains, including, for example, methoxyl, ethoxyl, propoxyl, isopropoxyl, butoxyl, t-butoxyl, and pentoxyl.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl.

“Dialkylamino” refers to an —NRR′ group wherein each of R and R′ is independently an alkyl group and/or a substituted alkyl group as previously described. Exemplary alkylamino groups include ethylmethylamino, dimethylamino, and diethylamino.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O—CO— group. Exemplary aryloxycarbonyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an H₂N—CO— group.

“Alkylcarbamoyl” refers to a R′RN—CO— group wherein one of R and R′ is hydrogen and the other of R and R′ is alkyl and/or substituted alkyl as previously described.

“Dialkylcarbamoyl” refers to a R′RN—CO— group wherein each of R and R′ is independently alkyl and/or substituted alkyl as previously described.

“Acyloxyl” refers to an acyl-O— group wherein acyl is as previously described.

“Acylamino” refers to an acyl-NH— group wherein acyl is as previously described.

“Aroylamino” refers to an aroyl-NH— group wherein aroyl is as previously described.

The term “amino” refers to the —NH₂ group.

The term “carbonyl” refers to the —(C═O)— group.

The term “carboxyl” refers to the —COOH group.

The term “cyano” refers to the —CN group.

The tennis “halo”, “halide”, or “halogen” as used herein refer to fluoro, chloro, bromo, and iodo groups.

The term “hydroxyl” refers to the —OH group.

The term “hydroxyalkyl” refers to an alkyl group substituted with an —OH group.

The term “mercapto” refers to the —SH group.

The term “oxo” refers to ═O.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term “cycloalkenyl” refers to a partially unsaturated cyclic hydrocarbon group containing one or more rings, for example, one ring, two rings, three rings, or four rings, with three or more carbon atoms per ring, for example, 3, 4, 5, 6, 7, or 8 carbon atoms per ring. Exemplary cycloalkenyl groups include, but are not limited to, cyclopropenyl, cyclobutenyl, cyclopentenyl, cyclohexenyl, and the like. Cycloalkenyl groups can be optionally substituted, such as with one or more substituents, e.g. 1, 2, 3, or 4 substituents, at any available point of attachment. Exemplary substituents include, but are not limited to, alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl.

The term “substituted cycloalkenyl” refers to a cycloalkenyl group substituted with one or more substituents, preferably 1, 2, 3, or 4 substituents, at any available point of attachment. Exemplary substituents include, but are not limited to, alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl.

When the term “independently selected” is used, the substituents being referred to (e.g., R groups, such as groups R¹ and R², or groups X and Y), can be identical or different. For example, both R¹ and R² can be substituted alkyls, or R¹ can be hydrogen and R² can be a substituted alkyl, and the like.

A named “R”, “R′,” “X,” “Y,” “Y′”, “A,” “A′”, “B,” “L,” or “Z” group will generally have the structure that is recognized in the art as corresponding to a group having that name, unless specified otherwise herein. For the purposes of illustration, certain representative “R,” “X,” and “Y” groups as set forth above are defined below. These definitions are intended to supplement and illustrate, not preclude, the definitions that would be apparent to one of ordinary skill in the art upon review of the present disclosure.

The term “treatment” as used herein covers any treatment of a disease and/or condition in an animal or mammal, particularly a human, and includes: (i) preventing a disease, disorder and/or condition and/or symptoms from occurring in a person which can be predisposed to the disease, disorder and/or condition, or at risk for being exposed to an agent that can cause the disease, disorder, and/or condition and/or symptoms; but, has not yet been diagnosed as having it; (ii) inhibiting the disease, disorder and/or condition, and/or symptoms i.e., arresting its development; and (iii) relieving the disease, disorder and/or condition, and/or symptoms i.e., causing regression of the disease, disorder and/or condition. iv) the augmentation of compensatory mechanisms, such as promotion of stem cells, that while not treating the primary disease can lead to reduced symptoms and improved function.

The term “mimetic” refers to a compound having similar functional and/or structural properties to another known compound or a particular fragment of that known compound, such as a known compound of biological origin, e.g., a polypeptide or fragment thereof.

“Binding specificity” refers to the ability of a protein or other type of molecule capable of recognizing and interacting with a complementary site on another protein or other type of molecule.

The term “pharmacophore”, as used herein, refers to a specific model or representation of a molecular moiety capable of exerting a selected biochemical effect, e.g., inhibition of an enzyme, binding to a receptor, chelation of an ion, and the like. A selected pharmacophore can have more than one biochemical effect, e.g., can be an inhibitor of one enzyme and an agonist of a second enzyme. A therapeutic agent can include one or more pharmacophores, which can have the same or different biochemical activities.

The term “derivative” as used herein refers to a compound chemically modified so as to differentiate it from a parent compound. Such chemical modifications can include, for example, replacement of hydrogen by an alkyl, acyl, or amino group. A derivative compound can be modified by, for example, glycosylation, pegylation, or any similar process that retains at least one biological or immunological function of the compound from which it was derived.

The term “hydrophilicity” is used in the common manner of the field as having an affinity for water; readily absorbing and/or dissolving in water.

The term “lipophilicity” is used in the common manner of the field as having an affinity for, tending to combine with, or capable of dissolving in lipids.

The term “amphipathicity”, as used herein, describes a structure having discrete hydrophobic and hydrophilic regions. Thus, one portion of the structure interacts favorably with aqueous and other polar media, while another portion of the structure interacts favorably with non-polar media.

The term “solubility” as used herein, describes the maximum amount of solute that will dissolve in a given amount of solvent at a specified temperature.

The term “bioavailability” as used herein refers to the systemic availability (i.e., blood/plasma levels) of a given amount of compound administered to a subject. The term further encompasses the rate and extent of absorption of compound that reaches the site of action.

As used herein, “solvate” means a complex formed by solvation (the combination of solvent molecules with molecules or ions of the active agent of the present invention), or an aggregate that consists of a solute ion or molecule (the active agent of the present invention) with one or more solvent molecules. Examples of hydrate include, but are not limited to, hemihydrate, monohydrate, dihydrate, trihydrate, hexahydrate, etc. It should be understood by one of ordinary skill in the art that the pharmaceutically acceptable salt of the present compound may also exist in a solvate form. The solvate is typically formed via hydration which is either part of the preparation of the present compound or through natural absorption of moisture by the anhydrous compound of the present invention. Solvates, including hydrates, may be found in stoichiometric ratios, for example, with two, three, four salt molecules per solvate or per hydrate molecule. Solvents used for crystallization, such as alcohols, especially methanol and ethanol; aldehydes; ketones, especially acetone; esters, e.g. ethyl acetate; may be embedded in the crystal grating.

The term “prodrug” as used herein refers to any compound that when administered to a biological system generates the drug substance (a biologically active compound) in or more steps involving spontaneous chemical reaction(s), enzyme catalyzed chemical reaction(s), or both. Standard prodrugs are formed using groups attached to functionality, e.g. HO—, HS—, HOOC—, R₂N—, associated with the drug substance that cleave in vivo. Prodrugs for these groups are well known in the art and are often used to enhance oral bioavailability or other properties beneficial to the formulation, delivery, or activity of the drug. Standard prodrugs include, but are not limited to, carboxylate esters where the group is alkyl, aryl, aralkyl, acyloxyalkyl, alkoxycarbonyloxyalkyl as well as esters of hydroxyl, thiol and amines where the group attached is an acyl group, an alkoxycarbonyl, aminocarbonyl, phosphate or sulfate.

Where the compounds of the present invention have at least one asymmetric center, they may accordingly exist as enantiomers. Where the compounds possess two or more asymmetric centers, they may additionally exist as diastereoisomers. It is to be understood that all such stereoisomers and mixtures thereof in any proportion are encompassed within the scope of the present invention. Where the compounds possess geometrical isomers, all such isomers and mixtures thereof in any proportion are encompassed within the scope of the present invention. Where so indicated in the claims herein, if a single enantiomer of the potentially optically active heterocyclic compounds disclosed is desired, for either health or efficacy reasons, preferably it is present in an enantiomeric excess of at least about 80%, or at least about 90%, or at least about 95%, or at least about 98%, or at least about 99%, or at least about 99.5%.

Tautomers of the compounds of the invention are encompassed by the present application. Thus, for example, a carbonyl includes its hydroxyl tautomer.

### Compounds of the Present Application

In one aspect, the present application discloses a compound having binding specificity for a p75^{NTR} molecule.

In some embodiments, the compound having binding specificity for a p75^{NTR} molecule is a mimetic of a neurotrophin β-turn loop.

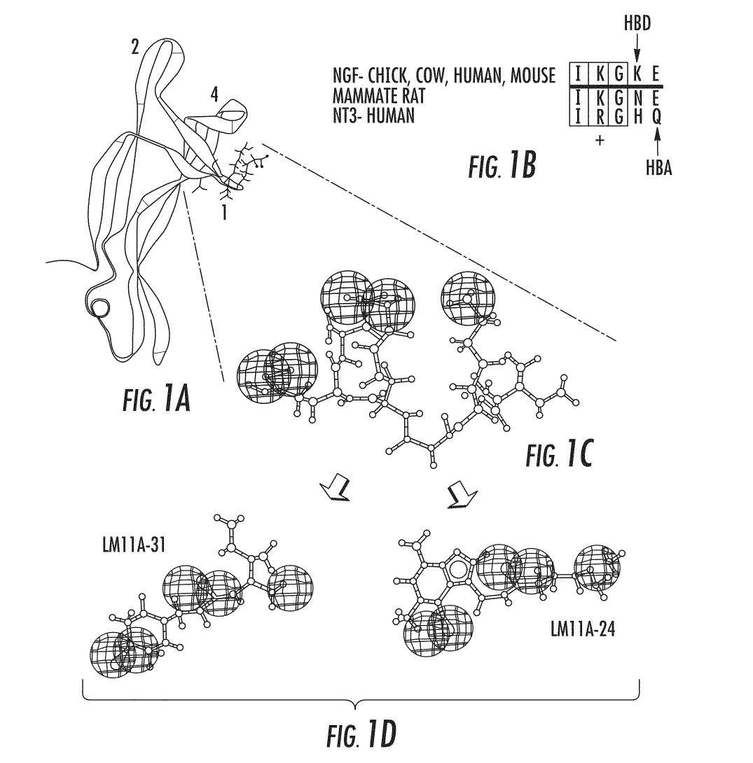

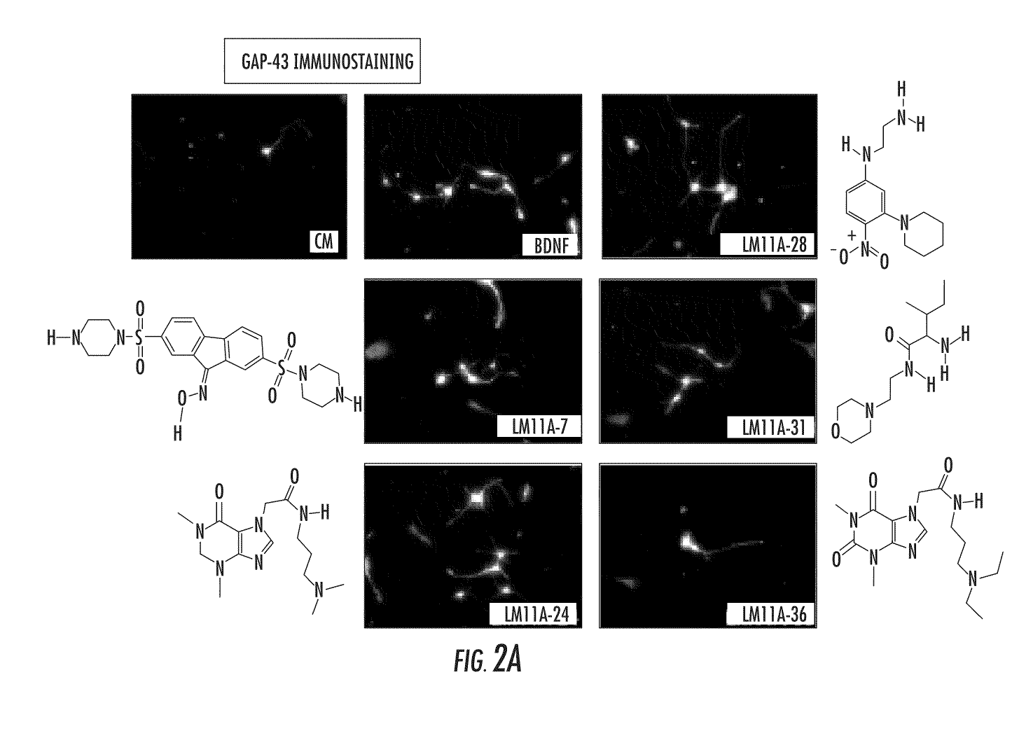

In some embodiments, the compound comprises a pharmacophore substantially identical to the pharmacophore illustrated in FIG. 1c.

In some embodiments, the compound is a small molecule or a peptide.

In one aspect, the present application discloses a compound selected from the group consisting of:

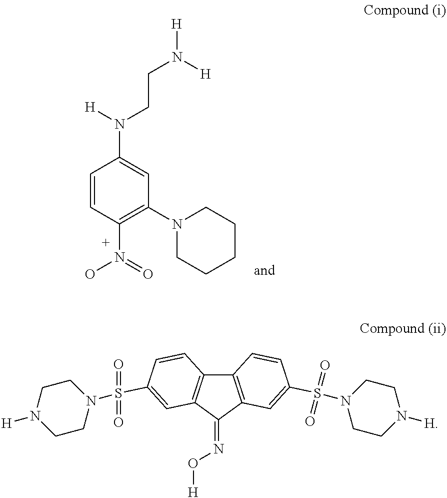

In another aspect, the present application discloses a compound is selected from the group consisting of a compound of Formula A or Formula B:

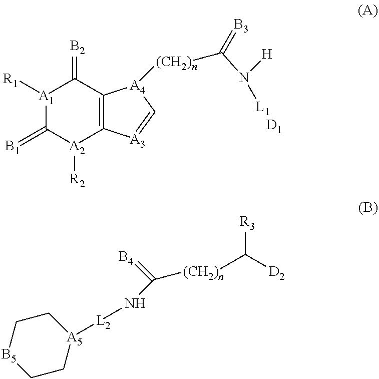

wherein:

n is an integer from 0 to 8;

L₁ and L₂ are a linking group selected from the group consisting of alkylene, substituted alkylene, cycloalkyl, substituted cycloalkyl, cycloalkene, substituted cycloalkene, aryl, substituted aryl, alkenylene, and substituted alkenylene;

R₁, R₂, and R₃ are each independently selected from the group consisting of H, alkyl, substituted alkyl, cycloalkyl, halo, cyano, nitro, mercapto, hydroxyl, alkoxyl, aryl, acyloxyl, substituted aryl, and aralkyloxyl;

A₁, A₂, A₃, A₄, and A₅ are each independently selected from the group consisting of N and CH;

B₁, B₂, B₃, B₄, and B₅ are each independently selected from the group consisting of O, S, and NR₄, wherein R₄ is selected from the group consisting of H, alkyl, substituted alkyl, cycloalkyl, aryl, and substituted aryl; and

D₁ and D₂ are selected from the group consisting of:

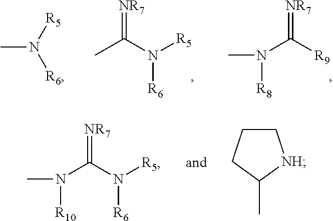

wherein:
each R₅, R₆, R₈, R₉, and R₁₀ is independently selected from the group consisting of H, alkyl, substituted alkyl, cycloalkyl, aryl, substituted aryl, aralkyl, hydroxyalkyl, hydroxycycloalkyl, alkoxycycloalkyl, aminoalkyl, acyloxyl, alkylaminoalkyl, and alkoxycarbonyl;
each R₇ is independently selected from the group consisting of H, hydroxyl, alkyl, substituted alkyl, aryl, substituted aryl, acyloxyl, and alkoxyl; or
R₇ and R₅ or R₇ and R₉ together represent a C₂ to C₁₀ alkyl, C₂ to C₁₀ hydroxyalkyl, or C₂ to C₁₀ alkene;
   or a pharmaceutically acceptable salt thereof.

In some embodiments, L₁ and L₂ are each independently —(CH₂)ₘ—, wherein m is an integer from 1 to 8.

In some embodiments, the compound of Formula (A) has the following structure:

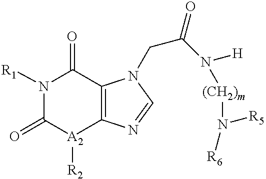

wherein:

m is an integer from 1 to 8;

R₁ and R₂ are each independently selected from the group consisting of H, alkyl, substituted alkyl, cycloalkyl, aryl, aryloxyl, substituted aryl, and aralkyloxyl; and

R₅ and R₆ are each independently selected from the group consisting of H, alkyl, substituted alkyl, cycloalkyl, aryl, substituted aryl, aralkyl, hydroxyl, alkoxyl, hydroxyalkyl, hydroxycycloalkyl, alkoxycycloalkyl, aminoalkyl, acyloxyl, alkylaminoalkyl, and alkoxycarbonyl.

In some embodiments, the compound of Formula (A) has the following structure:

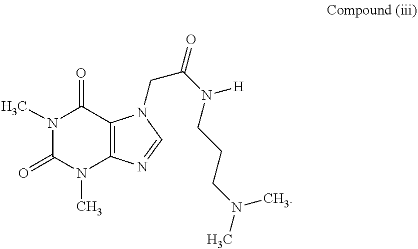 [SMILES: CN(C)CCCNC(=O)Cn1cnc2c1c(=O)n(C)c(=O)n2C]

In some embodiments, the compound of Formula (B) has the following structure:

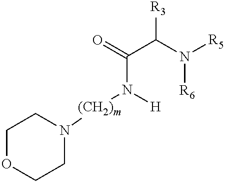

wherein:

m is an integer from 1 to 8;

R₃ is selected from the group consisting of H, alkyl, substituted alkyl, cycloalkyl, halo, hydroxyl, alkoxyl, aryl, aryloxyl, substituted aryl, and aralkyloxyl; and

R₅ and R₆ are each independently selected from the group consisting of H, alkyl, substituted alkyl, cycloalkyl, aryl, substituted aryl, aralkyl, hydroxyl, alkoxyl, hydroxyalkyl, hydroxycycloalkyl, alkoxycycloalkyl, aminoalkyl, acyloxyl, alkylaminoalkyl, and alkoxycarbonyl.

In some embodiments, the compound of Formula (B) has the following structure:

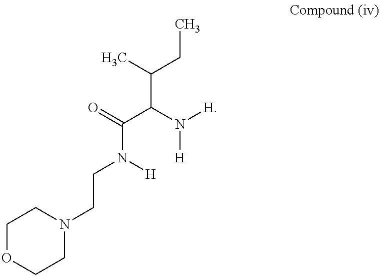

In some embodiments, the compound of Formula (A) is not:

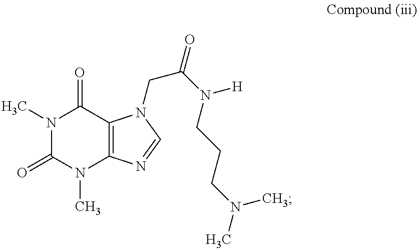 [SMILES: CN(C)CCCNC(=O)Cn1cnc2c1c(=O)n(C)c(=O)n2C]

and the compound of Formula (B) is not:

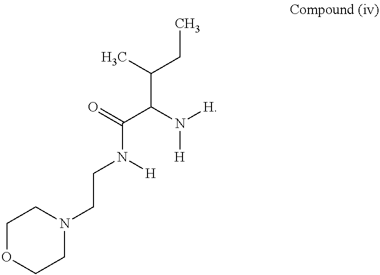

In some embodiments, the neurotrophin is a nerve growth factor (NGF). In some embodiments, the β-turn loop is loop 1 of the NGF.

In some embodiments, the compound has the formula:

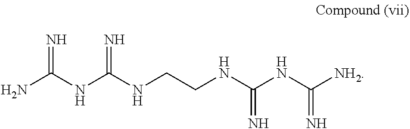 [SMILES: N=C(N)NC(=N)NCCNC(=N)NC(=N)N]

In some embodiments, the compound has binding specificity for a neurotrophin binding site of the p75^{NTR} molecule.

In some embodiments, the compound comprises a derivative of a parent compound having binding specificity for a p75^{NTR} molecule, wherein the derivative also has binding specificity for the p75^{NTR} molecule.

In some embodiments, the derivative exhibits an enhancement in at least one of the characteristics selected from the group consisting of hydrophilicity, lipophilicity, amphipathicity, solubility, bioavailability, and resistance to hepatic degradation, as compared to the parent compound.

In one aspect, the present application discloses a compound of Formula I:

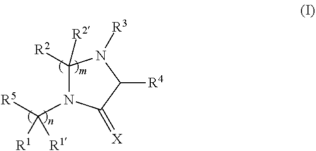

or a pharmaceutically acceptable salt, ester, prodrug or solvate thereof, wherein: each of R¹, R^{1′}, R², R^{2′}, R³, and R⁴ is independently hydrogen or optionally substituted alkyl; or R² and R^{2′} taken together form ═O, ═S or ═CH₂; R⁵ is heterocycloalkyl; X is CH₂, NH, O or S; n is 0, 1, 2, 3, 4, or 5; and m is 1 or 2. In another aspect, in a compound of Formula I each of R¹, R^{1′}, R², R^{2′}, R³, and R⁴ is independently hydrogen, optionally substituted alkyl, optionally substituted alkenyl or optionally substituted alkynyl; or R² and R^{2′} taken together form ═O or ═S; R⁵ is heterocycloalkyl; X is O or S; n is 0, 1, 2, 3, or 4; and m is 1 or 2. In one embodiment of any of the disclosed aspects, X is O; and m is 1. In another embodiment R² and R^{2′} taken together form ═O; and each of R³ and R⁴ is independently optionally substituted C₁-C₆ alkyl. In another embodiment, R⁵ is morpholinyl, thiomorpholinyl, tetrahydro-2H-pyran, 1-methylpiperazinyl, piperidinyl, or pyrrolidinyl; and each of R¹ and R^{1′} is independently hydrogen or optionally substituted C₁-C₄ alkyl. In another embodiment, X is O; m is 1; R² and R^{2′} taken together form ═O; each of R³ and R⁴ is independently optionally substituted C₁-C₆ alkyl; R⁵ is morpholinyl, thiomorpholinyl, tetrahydro-2H-pyran, 1 methylpiperazinyl, piperidinyl, or pyrrolidinyl; and each of R¹ and R^{1′} is independently hydrogen or optionally substituted C₁-C₄ alkyl. In yet another embodiment, m is 2; X is 0; R² and R^{2′} each is hydrogen; R³ is optionally substituted C₁-C₄ alkyl; R⁵ is a nitrogen-bound morpholinyl, 1-methylpiperazinyl, piperidinyl, or pyrrolidinyl; and each of R¹ and R^{1′} is independently hydrogen or optionally substituted C₁-C₄ alkyl. In another aspect, the compound has the structure of Formula IA:

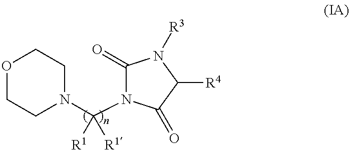

wherein each of R¹, R^{1′}, R³, and R⁴ independently is hydrogen or optionally substituted alkyl; and n is 0, 1, 2, 3, 4, or 5. Another aspect is the compound having the structure of Formula IA wherein each of R¹, R^{1′}, R³, and R⁴ independently is hydrogen, optionally substituted alkyl, optionally substituted alkenyl or optionally substituted alkynyl; and n is 1, 2, 3, or 4. In one embodiment of any of the disclosed aspects, n is 2; each of R¹ and R^{1′} is hydrogen; R³ is methyl and R⁴ is sec-butyl. In another embodiment, R⁵ is a heterocycloalkyl bound via a heteroatom; m is 2; and X is 0. In another embodiment, R² and R^{2′} each is hydrogen; and R³ is optionally substituted C₁-C₄ alkyl. In yet another embodiment, R⁵ is a nitrogen-bound morpholinyl, 1-methylpiperazinyl, piperidinyl, or pyrrolidinyl; and each of R¹ and R^{1′} is independently hydrogen or optionally substituted C₁-C₄ alkyl. In still another variation, the compound has the structure of Formula IB:

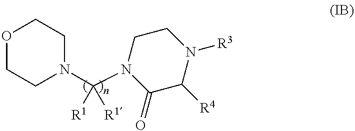

One aspect is a compound having the structure of Formula IB wherein each of R¹, R^{1′}, R³, and R⁴ independently is hydrogen, optionally substituted alkyl, optionally substituted alkenyl or optionally substituted alkynyl; and n is 1, 2, 3, or 4. In one embodiment of any of the disclosed aspects, n is 2; each of R¹ and R^{1′} is hydrogen; R³ is methyl and R⁴ is sec-butyl. In another embodiment, R⁵ is a heterocycloalkyl bound via a heteroatom; m is 2; and X is 0. In another embodiment, R² and R^{2′} each is hydrogen; and R³ is optionally substituted C₁-C₄ alkyl. In yet another embodiment, R⁵ is a nitrogen-bound morpholinyl, 1-methylpiperazinyl, piperidinyl, or pyrrolidinyl; and each of R¹ and R^{1′} is independently hydrogen or optionally substituted C₁-C₄ alkyl.

In another aspect, the present application discloses a compound of Formula II:

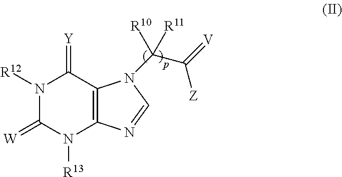

or a pharmaceutically acceptable salt, ester, prodrug or solvate thereof, wherein p is 0, 1, 2, 3, 4, 5, or 6; each of Y, V, and W is independently ═CH₂, ═NH, ═O or ═S; each of R¹⁰ and R¹¹ is independently hydrogen or optionally substituted alkyl; each of R¹² and R¹³ is independently hydrogen, —NR^{a}R^{b}, —OH, —C(═O)OR^{a}, —C(═O)NHR^{a}, —NHC(═O)R^{a}, —NHS(═O)₂R^{a}, or optionally substituted alkyl; each of R^{a} and R^{b} is independently hydrogen or optionally substituted alkyl; and Z is heterocycloalkyl or heteroaryl wherein each heterocycloalkyl or heteroaryl is bound via a heteroatom and is optionally substituted. Another aspect is a compound of Formula II wherein p is 1, 2, 3, 4, or 5; each of Y, V, and W is independently ═O or ═S; each of R¹⁰ and R¹¹ is independently hydrogen, optionally substituted alkyl, optionally substituted alkenyl or optionally substituted alkynyl; each of R¹² and R¹³ is independently hydrogen, —NR^{a}R^{b}, —OH, optionally substituted alkyl, optionally substituted alkenyl or optionally substituted alkynyl; each of R^{a} and R^{b} is independently hydrogen or optionally substituted alkyl; and Z is heterocycloalkyl or heteroaryl wherein each heterocycloalkyl or heteroaryl is bound via a heteroatom and is optionally substituted. In one embodiment of any of the disclosed aspects, p is 1, 2 or 3; each of Y, V, and W is O; and each of R¹² and R¹³ is independently hydrogen or optionally substituted C₁-C₄ alkyl; and Z is an optionally substituted nitrogen-bound heterocycloalkyl. In another embodiment, p is 1; each of R¹⁰ and R¹¹ is hydrogen; and each of R¹² and R¹³ is independently C₁-C₄ alkyl.

In another embodiment, the compound has the structure of Formula IIA:

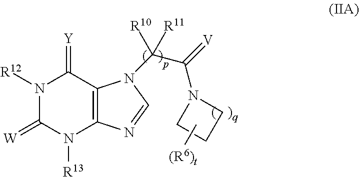

or a pharmaceutically acceptable salt, ester, prodrug or solvate thereof, wherein p is 0, 1, 2, 3, 4, 5, or 6; q is 1, 2, 3, or 4; t is 0, 1, 2, or 3; each of Y, V, and W is independently O or S; each of R¹⁰ and R¹¹ is independently hydrogen or optionally substituted alkyl; each of R¹² and R¹³ is independently hydrogen, —NR^{a}R^{b}, —OH, —C(═O)OR^{a}, —C(═O)NHR^{a}, —NHC(═O)R^{a}, —NHS(═O)₂R^{a}, or optionally substituted alkyl; each of R⁶ is independently —NR^{a}R^{b}, —OH, —C(═O)OR^{a}, —C(═O)NHR^{a}, —NHC(═O)R^{a}, —NHS(═O)₂R^{a}, or optionally substituted alkyl; and each of R^{a} and R^{b} is independently hydrogen or optionally substituted alkyl. In one embodiment, q is 1, 2, 3, or 4; t is 0, 1, 2, or 3; each of Y, V, and W is independently O or S; and each of R⁶ is independently —NR^{a}R^{b}, —OH, —C(═O)OR^{a}, —C(═O)NHR^{a}, —NHC(═O)R^{a}, —NHS(═O)₂R^{a}, or optionally substituted alkyl. In yet another aspect, the compound has the structure of Formula IIA wherein p is 1, 2, 3, 4, or 5; q is 2 or 3; t is 0, 1, 2, or 3; each of Y, V, and W is independently O or S; each of R¹⁰ and R¹¹ is independently hydrogen, optionally substituted alkyl, optionally substituted alkenyl, or optionally substituted alkynyl; each of R⁶ is independently —NR^{a}R^{b}, —OH, or optionally substituted alkyl; and each of R^{a} and R^{b} is independently hydrogen or optionally substituted alkyl. In another embodiment of any of the disclosed aspects, each of Y, V, and W is O; q is 1; each of R¹⁰ and R¹¹ is independently hydrogen or C₁-C₄ alkyl; and each of R¹² and R¹³ is independently C₁-C₄ alkyl. In yet another embodiment, each of R¹⁰ and R¹¹ is independently hydrogen; each of R¹² and R¹³ is independently -Me; and each of R⁶, R^{6′}, R⁷, R^{7′}, R⁸, R^{8′}, R⁹, and R^{9′} is independently hydrogen, —NR^{a}R^{b}, —OH, or optionally substituted alkyl. In still a further variation of any of the disclosed embodiments, each of R¹⁰ and R¹¹ is independently —H; each of R¹² and R¹³ is independently -Me; q is 2; and each of R⁶, R^{6′}, R⁷, R^{7′}, R⁸, R^{8′}, R⁹, and R^{9′} is independently hydrogen, —NR^{a}R^{b}, —OH, or optionally substituted alkyl. In another further variation of any of the disclosed embodiments, each of R⁶, R^{6′}, R⁷, R^{7′}, R⁸, R^{8′}, and R⁹ is hydrogen; and R^{9′} is —N(CH₃)₂.

In another embodiment, the compound has the structure of Formula IIB:

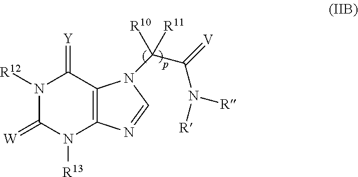

or a pharmaceutically acceptable salt, ester, prodrug or solvate thereof, wherein p is 0, 1, 2, 3, 4, 5, or 6; each of Y, V, and W is independently O or S; each of R¹⁰ and R¹¹ is independently hydrogen or optionally substituted alkyl; each of R¹² and R¹³ is independently hydrogen, —NR^{a}R^{b}, —OH, —C(═O)OR^{a}, —C(═O)NHR^{a}, —NHC(═O)R^{a}, —NHS(═O)₂R^{a}, or optionally substituted alkyl; and R′ and R″ taken together with the nitrogen to which they are attached form an optionally substituted heterocyclic aryl. In yet another aspect, the compound has the structure of Formula IIB wherein p is 1, 2, 3, 4, or 5; each of Y, V, and W is independently O or S; each of R¹⁰ and R¹¹ is independently hydrogen, optionally substituted alkyl, optionally substituted alkenyl, or optionally substituted alkynyl; R′ and R″ taken together with the nitrogen to which they are attached form a an optionally substituted pyridyl, an optionally substituted pyrrolyl, an optionally substituted pyrimidyl or an optionally substituted pyrazinyl. In another embodiment of any of the disclosed aspects, each of Y, V, and W is O; each of R¹⁰ and R¹¹ is independently hydrogen or C₁-C₄ alkyl; and each of R¹² and R¹³ is independently C₁-C₄ alkyl. In yet another embodiment, each of R¹⁰ and R¹¹ is independently hydrogen; each of R¹² and R¹³ is independently -Me. In still a further variation of any of the disclosed embodiments, each of R¹⁰ and R¹¹ is independently —H; each of R¹² and R¹³ is independently -Me; q is 2; and R′ and R″ taken together with the nitrogen to which they are attached form an optionally substituted pyrrolyl. In another further variation, the compound has the structural formula

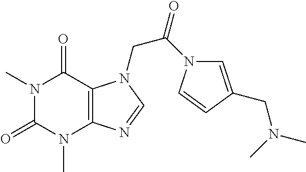

In another aspect, the present application discloses a compound of Formula III:

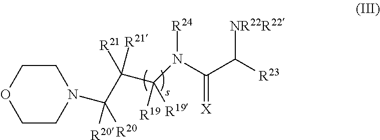

or a pharmaceutically acceptable salt, ester, prodrug or solvate thereof, wherein X is CH₂, NH, O or S; s is 0, 1, 2, 3 or 4; each of R¹⁹, R^{19′}, R²⁰, R^{20′}, R²¹, R^{21′}, R²², R^{22′} and R²⁴ is independently absent, hydrogen or optionally substituted alkyl; or R²⁰ and R^{20′} taken together form ═O, ═S, or ═CH₂; or R²⁰ and R²¹ taken together with the atoms to which they are attached form an optionally substituted cycloalkyl; or R²⁰ and R²¹ taken together with the atoms to which they are attached form an optionally substituted aryl; or R¹⁹ and R²⁰ taken together with the atoms to which they are attached form an optionally substituted cycloalkyl; or R¹⁹ and R²⁰ taken together with the atoms to which they are attached form an optionally substituted aryl; and R²³ is optionally substituted alkyl, optionally substituted cycloalkyl or optionally substituted aryl. In one embodiment of any of the disclosed aspects or variations, R²³ is hydrogen, optionally substituted alkyl, optionally substituted cycloalkyl or optionally substituted aryl. In one embodiment of any of the disclosed aspect or variations, the compound of Formula III is not 2-amino-3-methyl-N-(2-morpholinoethyl)-butanamide. Yet another aspect is a compound of Formula III wherein X is O or S; s is 0, 1, 2, or 3; each of R¹⁹, R^{19′}, R²⁰, R^{20′}, R²¹, R^{21′}, R²², R^{22′} and R²⁴ is independently absent, hydrogen, optionally substituted alkyl, optionally substituted alkenyl or optionally substituted alkynyl; or R²⁰ and R^{20′} taken together form ═O, or ═S; or R²⁰ and R²¹ taken together with the atoms to which they are attached form an optionally substituted cycloalkyl; or R²⁰ and R²¹ taken together with the atoms to which they are attached form an optionally substituted aryl; or R¹⁹ and R²⁰ taken together with the atoms to which they are attached form an optionally substituted cycloalkyl; or R¹⁹ and R²⁰ taken together with the atoms to which they are attached form an optionally substituted aryl; and R²³ is optionally substituted alkyl, optionally substituted alkenyl or optionally substituted alkynyl optionally substituted cycloalkyl or optionally substituted aryl. In one embodiment of any of the disclosed aspects, X is O; s is 0; R^{22′} is hydrogen; and R²² is optionally substituted C₁-C₆ alkyl. In another embodiment, each of R²⁰, R^{20′}, R²¹, and R^{21′} is independently hydrogen or optionally substituted C₁-C₄ alkyl; or R²⁰ and R^{20′} taken together form ═O. In another embodiment, X is O; s is 0; each of R²² and R^{22′} is hydrogen or optionally substituted C₁-C₆ alkyl; and each of R²⁰, R^{20′}, R²¹, and R^{21′} is independently hydrogen or optionally substituted C₁-C₄ alkyl; or R²⁰ and R^{20′} taken together form ═O. In another embodiment R²⁰ and R²¹ taken together with the atoms to which they are attached form an optionally substituted cycloalkyl; or R²⁰ and R²¹ taken together with the atoms to which they are attached form an optionally substituted aryl. In one variation, the compound has the structural formula

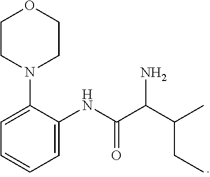 [SMILES: CCC(C)C(N)C(=O)Nc1ccccc1N1CCOCC1]

In another embodiment, s is 2; X is O; R¹⁹ and R²⁰ taken together with the atoms to which they are attached form an optionally substituted cycloalkyl; or R¹⁹ and R²⁰ taken together with the atoms to which they are attached form an optionally substituted aryl. In one variation, the compound has the structural formula:

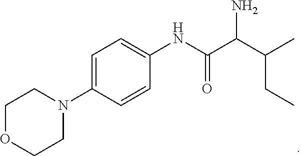 [SMILES: CCC(C)C(N)C(=O)Nc1ccc(N2CCOCC2)cc1]

In another variation, the compound has a structural formula selected from the group consisting of:

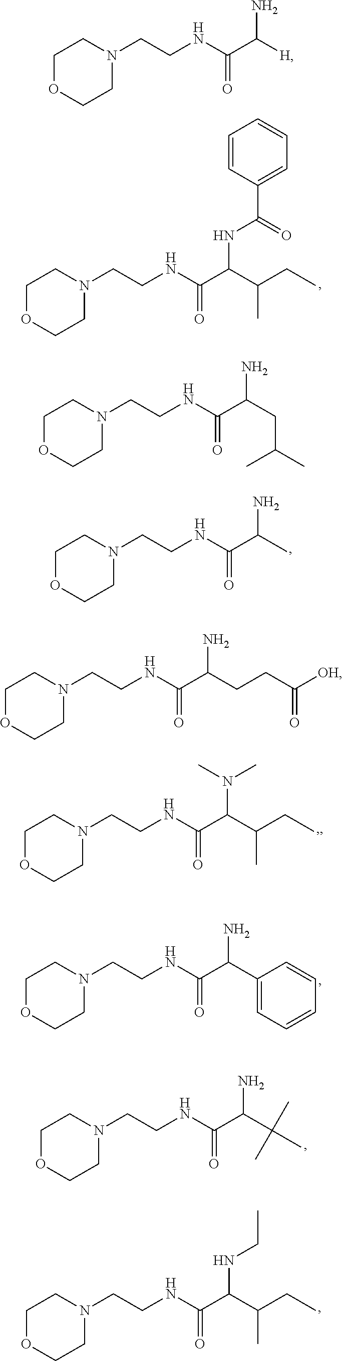

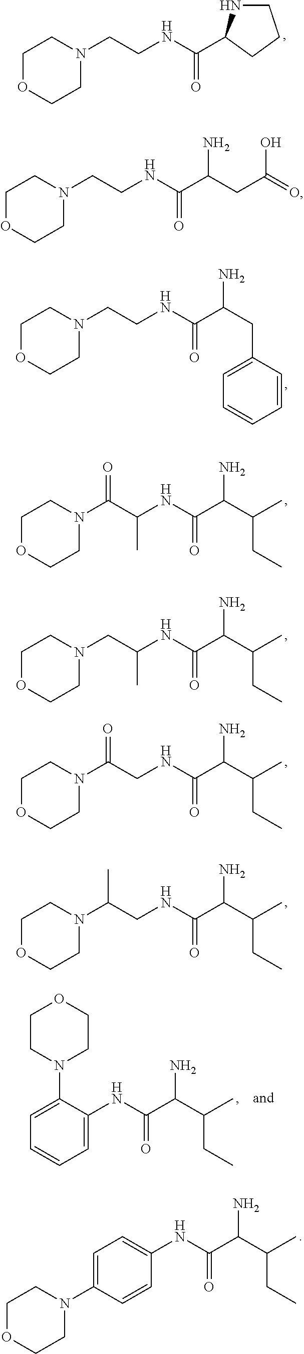

In yet another variation of any of the disclosed embodiments, the compound has a structural formula selected from the group consisting of:

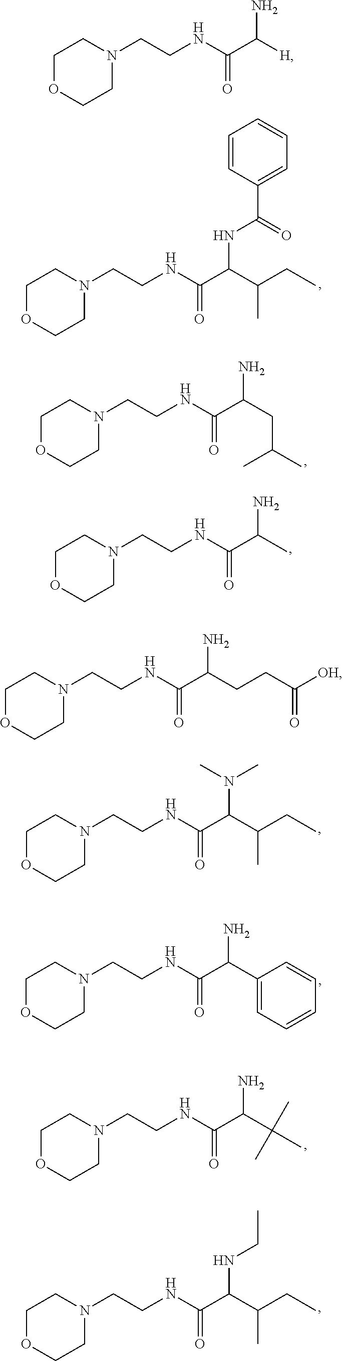

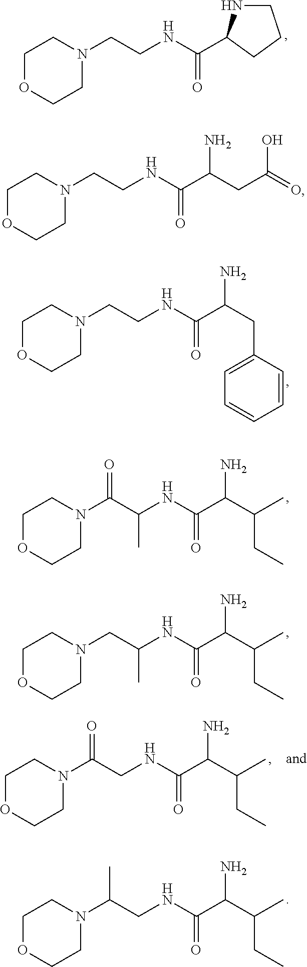

Further included is a compound having a structural formula selected from the group consisting of

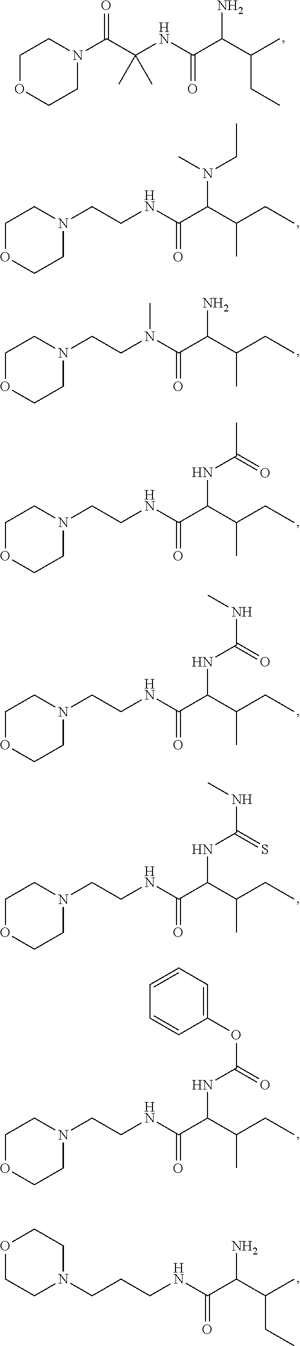

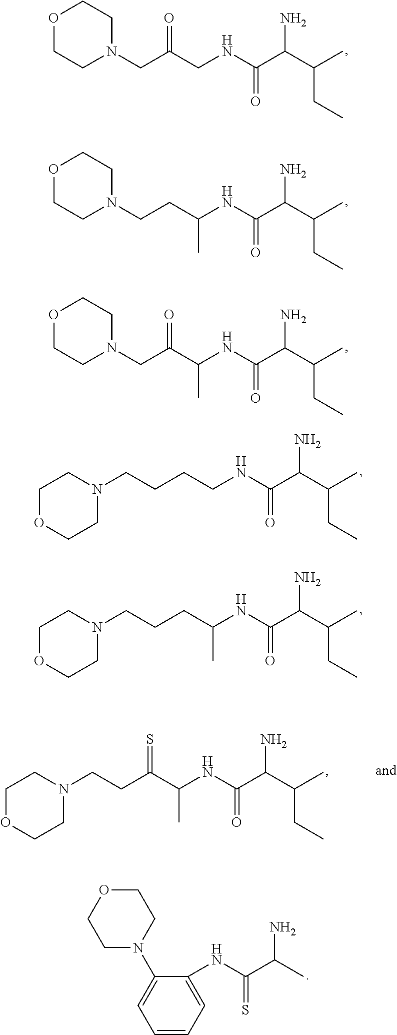

Such compounds can be prepared based on the disclosure herein and chemical synthetic routes familiar to one of skill in the art.

In still another aspect, the present application discloses a compound of Formula IV:

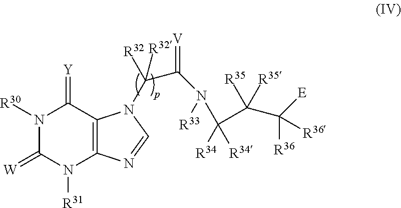

or a pharmaceutically acceptable salt, ester, prodrug or solvate thereof, wherein p is 1, 2, 3, 4, 5, or 6; each of Y, V, and W is independently CH₂, NH, O or S; each of R³⁰, R³¹, R³², R^{32′} R³³, R³⁴, R^{34′}, R³⁵, R^{35′}, R³⁶, and R^{36′} is independently absent, hydrogen or optionally substituted alkyl; or R³⁴ and R³⁶ taken together with the atoms to which they are attached form an optionally substituted carbocyclic ring; E is —CHR^{c}R^{d}, —NR^{c}R^{d}, —OR^{c}, and —SR^{c}; and each of R^{c} and R^{d} is independently hydrogen or optionally substituted alkyl; or R^{c} and R^{d} taken together with the nitrogen atom to which they are attached form an optionally substituted heterocyclic ring or R^{c} and R^{d} taken together with the carbon atom to which they are attached form an optionally substituted carbocyclic ring. In one embodiment of any of the disclosed aspects or variations, the compound of Formula IV is not N-(3-(diethylamino)propyl)-2-(4,6-dimethyl-5,7-dioxo-4,5,6,7-tetrahydro-1H-benzo[d]imidazol-1-yl)acetamide. In one variation, p is 1, 2, or 3; each of Y, V, and W is O or S; each of R³⁰ and R³¹ is independently optionally substituted C₁-C₄ alkyl; each of R³², R^{32′} R³³, R³⁴, R^{34′}, R³⁵, R^{35′}, R³⁶, and R^{36′} is independently hydrogen or optionally substituted C₁-C₄ alkyl; and E is —OR^{c}, —SR^{c}, or —NR^{c}R^{d} wherein R^{c} and R^{d} taken together with the nitrogen atom to which they are attached form an optionally substituted heterocycloalkyl. In one aspect, the compound has the structure of Formula IV wherein p is 1, 2, 3, or 4; each of Y, V, and W is independently O or S; each of R³⁰, R³¹, R³², R^{32′} R³³, R³⁴, R^{34′}, R³⁵, R^{35′}, R³⁶, and R^{36′} is independently absent, hydrogen, optionally substituted alkyl, optionally substituted alkenyl or optionally substituted alkynyl; or R³⁴ and R³⁶ taken together with the atoms to which they are attached form an optionally substituted carbocyclic ring; E is —CHR^{c}R^{d}, —NR^{c}R^{d}, —OR^{c}, or —SR^{c}; and each of R^{c} and R^{d} is independently hydrogen, optionally substituted alkyl, optionally substituted alkenyl or optionally substituted alkynyl; or R^{c} and R^{d} taken together with the nitrogen atom to which they are attached form an optionally substituted heterocyclic ring or R^{c} and R^{d} taken together with the carbon atom to which they are attached form an optionally substituted carbocyclic ring. In one embodiment of any of the disclosed aspects, p is 1, 2, or 3; each of Y, V, and W is O or S; E is —OR^{c} or —SR^{c}; each of R³², R^{32′} R³³, R^{34′}, R³⁵, R^{35′}, and R^{36′} is independently hydrogen; and each of R³⁰ and R³¹ is independently optionally substituted C₁-C₄ alkyl. In another embodiment, p is 1, 2, or 3; each of Y, V, and W is O or S; E is NR^{c}R^{d} and R^{c} and R^{d} taken together with the nitrogen atom to which they are attached form an optionally substituted heterocycloalkyl; each of R³⁰ and R³¹ is independently optionally substituted C₁-C₄ alkyl; and each of R³², R^{32′} R³³, R³⁴, R^{34′}, R³⁵, R^{35′}, R³⁶, and R^{36′} is independently hydrogen or optionally substituted C₁-C₄ alkyl. In yet another embodiment, p is 1; each of Y, V, and W is O; each of R³⁰ and R³¹ is independently —CH₃; R³³ is hydrogen; and each of R³², R^{32′} R³⁴, R^{34′}, R³⁵, R^{35′}, R³⁶, and R^{36′} is independently hydrogen or C₁-C₄ alkyl. In one variation of any of the disclosed embodiments, E is —NR^{c}R^{d} and each of R^{c} and R^{d} is independently hydrogen or optionally substituted alkyl. In another embodiment, R³⁴ and R³⁶ taken together with the atoms to which they are attached form an optionally substituted cycloalkyl; or R³⁴ and R³⁶ taken together with the atoms to which they are attached form an optionally substituted carbocyclic aryl. In another embodiment, the compound has the structural formula:

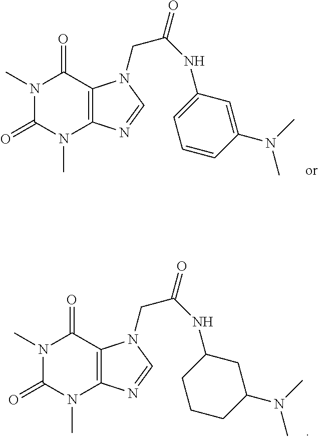

In another variation of any of the disclosed embodiments, E is —NR^{c}R^{d} and R^{c} and R^{d} taken together with the nitrogen atom to which they are attached form an optionally substituted heterocycloalkyl. In one variation, the compound has a structural formula selected from the group consisting of

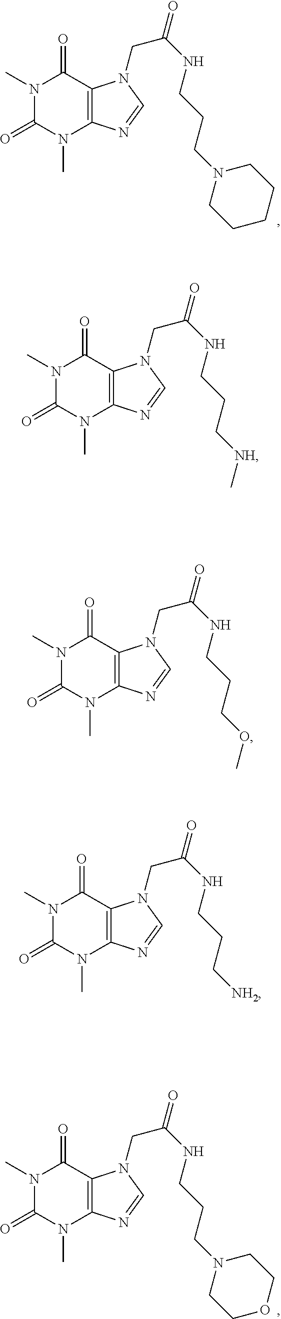 [SMILES: Cn1c(=O)c2c(ncn2CC(=O)NCCCN2CCCCC2)n(C)c1=O ; CNCCCNC(=O)Cn1cnc2c1c(=O)n(C)c(=O)n2C ; COCCCNC(=O)Cn1cnc2c1c(=O)n(C)c(=O)n2C ; Cn1c(=O)c2c(ncn2CC(=O)NCCCN)n(C)c1=O ; Cn1c(=O)c2c(ncn2CC(=O)NCCCN2CCOCC2)n(C)c1=O]

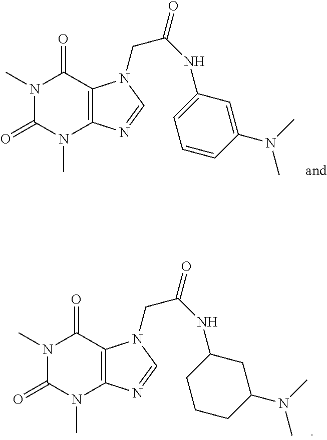

In yet another embodiment, the compound has a structural formula selected from the group consisting of:

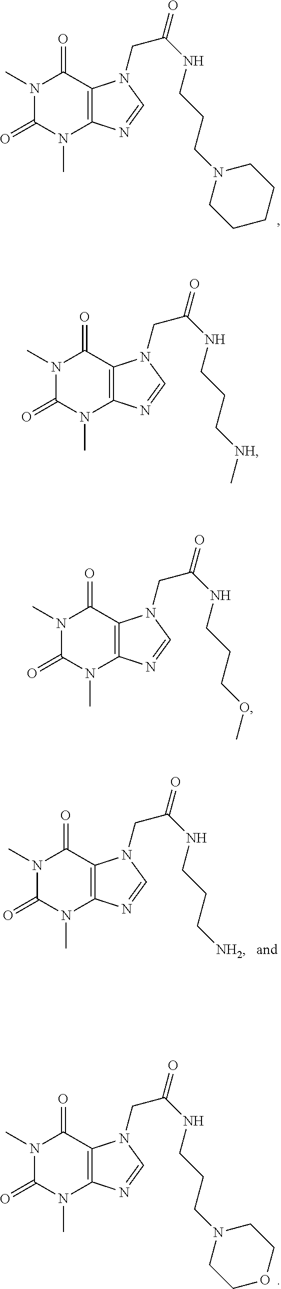 [SMILES: Cn1c(=O)c2c(ncn2CC(=O)NCCCN2CCCCC2)n(C)c1=O ; CNCCCNC(=O)Cn1cnc2c1c(=O)n(C)c(=O)n2C ; COCCCNC(=O)Cn1cnc2c1c(=O)n(C)c(=O)n2C ; Cn1c(=O)c2c(ncn2CC(=O)NCCCN)n(C)c1=O ; "and" ; Cn1c(=O)c2c(ncn2CC(=O)NCCCN2CCOCC2)n(C)c1=O]

Alternately, such compounds include:

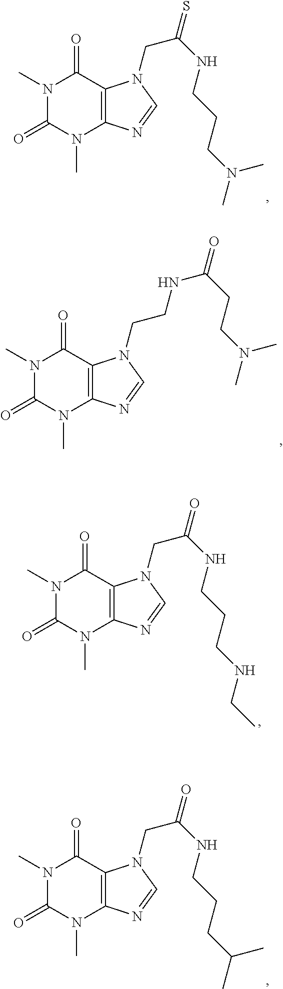

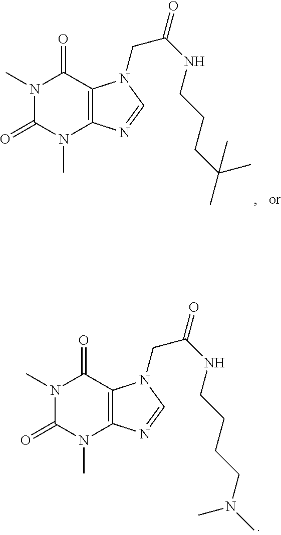

Still further included is included:

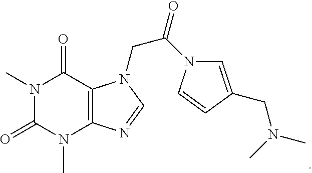

In another aspect, the present application provides a compound of Formula IVA:

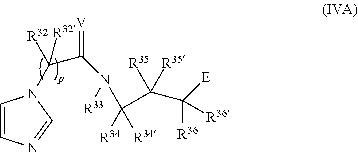

or a pharmaceutically acceptable salt, ester, prodrug or solvate thereof, wherein p is 1, 2, 3, 4, 5, or 6; V is CH₂, NH, O or S; each of R³², R^{32′} R³³, R³⁴, R^{34′}, R³⁵, R^{35′}, R³⁶, and R^{36′} is independently absent, hydrogen or optionally substituted alkyl; or R³⁴ and R³⁶ taken together with the atoms to which they are attached form an optionally substituted carbocyclic ring; E is —CHR^{c}R^{d}, —NR^{c}R^{d}, —OR^{c}, and —SR^{c}; and each of R^{c} and R^{d} is independently hydrogen or optionally substituted alkyl; or R^{c} and R^{d} taken together with the nitrogen atom to which they are attached form an optionally substituted heterocyclic ring or R^{c} and R^{d} taken together with the carbon atom to which they are attached form an optionally substituted carbocyclic ring. In one aspect, the compound has the structure of Formula IVA wherein p is 1, 2, 3, or 4; V is O or S; each of R³², R^{32′} R³³, R³⁴, R^{34′}, R³⁵, R^{35′}, R³⁶, and R^{36′} is independently absent, hydrogen, optionally substituted alkyl, optionally substituted alkenyl or optionally substituted alkynyl; or R³⁴ and R³⁶ taken together with the atoms to which they are attached form an optionally substituted carbocyclic ring; E is —CHR^{c}R^{d}, —OR^{c}, or —SR^{c}; and each of R^{c} and R^{d} is independently hydrogen, optionally substituted alkyl, optionally substituted alkenyl or optionally substituted alkynyl; or R^{c} and R^{d} taken together with the nitrogen atom to which they are attached form an optionally substituted heterocyclic ring or R^{c} and R^{d} taken together with the carbon atom to which they are attached form an optionally substituted carbocyclic ring. In one embodiment of any of the disclosed aspects, c is 1, 2, or 3; V is O or S; E is —OR^{c} or —SR^{c}; each of R³², R^{32′} R³³, R^{34′}, R³⁵, R^{35′}, and R^{36′} is independently hydrogen. In another embodiment, p is 1, 2, or 3; V is O or S; E is NR^{c}R^{d} and R^{c} and R^{d} taken together with the nitrogen atom to which they are attached form an optionally substituted heterocycloalkyl; and each of R³², R^{32′} R³³, R³⁴, R^{34′}, R³⁵, R^{35′}, R³⁶, and R^{36′} is independently hydrogen or optionally substituted C₁-C₄ alkyl. In yet another embodiment, p is 1; V is O; R³³ is hydrogen; and each of R³², R^{32′} R³⁴, R^{34′}, R³⁵, R^{35′}, R³⁶, and R^{36′} is independently hydrogen or C₁-C₄ alkyl. In one variation of any of the disclosed embodiments, E is —NR^{c}R^{d} and each of R^{c} and R^{d} is independently hydrogen or optionally substituted alkyl. In another embodiment, R³⁴ and R³⁶ taken together with the atoms to which they are attached form an optionally substituted cycloalkyl; or R³⁴ and R³⁶ taken together with the atoms to which they are attached form an optionally substituted carbocyclic aryl. In one embodiment, the compound has the structural formula

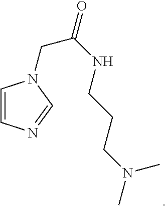

Since the p75 receptor is upregulated in various pathological states, compounds disclosed herein can also be linked to molecular markers that can be detected by imaging or other modalities. Such conjugates can be prepared according to synthetic methods known to those of skill in the art and applied in diagnostic strategies designed to detect such pathological states.

In another aspect, the present invention provides a compound selected from the group consisting of (2R,3R)-2-amino-3-methyl-N-(2-morpholinoethyl)-pentanamide; (2R,3S)-2-amino-3-methyl-N-(2-morpholinoethyl)-pentanamide; and (2S,3R)-2-amino-3-methyl-N-(2-morpholinoethyl)-pentanamide; or a pharmaceutically acceptable salt, solvate, ester, or prodrug thereof. In one embodiment, the compound is in a purity of about 80% or more. In another embodiment, the compound is in a purity of about 85% or more. In another embodiment, the compound is in a purity of about 90% or more. In another embodiment, the compound is in a purity of about 95% or more. In another embodiment, the compound is in a purity of about 96% or more. In another embodiment, the compound is in a purity of about 97% or more. In another embodiment, the compound is in a purity of about 98% or more. In another embodiment, the compound is in a purity of about 99% or more. In another embodiment, the compound is in a purity of about 99.5% or more.

In another aspect, the present invention provides a mixture of two or more compounds selected from the group consisting of (2S,3S)-2-amino-3-methyl-N-(2-morpholinoethyl)-pentanamide; (2R,3R)-2-amino-3-methyl-N-(2-morpholinoethyl)-pentanamide; (2R,3S)-2-amino-3-methyl-N-(2-morpholinoethyl)-pentanamide; and (2S,3R)-2-amino-3-methyl-N-(2-morpholinoethyl)-pentanamide; or a pharmaceutically acceptable salt, solvate, ester, or prodrug thereof, with the proviso that when the mixture consists of (2S,3S)-2-amino-3-methyl-N-(2-morpholinoethyl)-pentanamide and (2R,3R)-2-amino-3-methyl-N-(2-morpholinoethyl)-pentanamide, or a pharmaceutically acceptable salt, solvate, ester, or prodrug thereof; then (2S,3S)-2-amino-3-methyl-N-(2-morpholinoethyl)-pentanamide, or a pharmaceutically acceptable salt, solvate, ester, or prodrug thereof, is in an amount not less than about 5% by weight. In one embodiment, the mixture consists of any two of the aforementioned four compounds. In another embodiment, the mixture consists of any three of the aforementioned four compounds. In another embodiment, the mixture consists of the aforementioned four compounds. Subject to the above-mentioned proviso, the individual compounds in the mixture can be in any ratio or weight percentage. In one embodiment, any of the two or more compounds in the mixture is in an amount of about 0.5% by weight or more. In another embodiment, any of the two or more compounds in the mixture is in an amount of about 5% by weight or more. In another embodiment, each of the two or more compounds in the mixture is in an approximately equal amount.

Scheme A provides the chemical structures of the above-mentioned compounds.

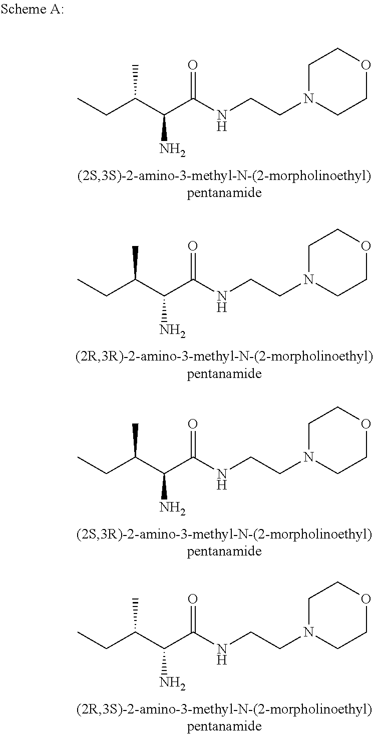 [SMILES: CC[C@H](C)[C@H](N)C(=O)NCCN1CCOCC1 ; CC[C@@H](C)[C@@H](N)C(=O)NCCN1CCOCC1 ; CC[C@@H](C)[C@H](N)C(=O)NCCN1CCOCC1 ; CC[C@H](C)[C@@H](N)C(=O)NCCN1CCOCC1]

In one embodiment, the present invention provides a mixture of (2R,3R)-2-amino-3-methyl-N-(2-morpholinoethyl)-pentanamide and (2S,3S)-2-amino-3-methyl-N-(2-morpholinoethyl)-pentanamide, or a pharmaceutically acceptable salt, solvate, ester, or prodrug thereof, with the proviso that (2S,3S)-2-amino-3-methyl-N-(2-morpholinoethyl)-pentanamide, or a pharmaceutically acceptable salt, solvate, ester, or prodrug thereof, is in an amount not less than about 5% by weight based on the total amount of the mixture. Subject to the above-mentioned proviso, the individual compounds in the mixture can be in any ratio or weight percentage. In one specific embodiment, the mixture consists of (2R,3R)-2-amino-3-methyl-N-(2-morpholinoethyl)-pentanamide and (2S,3S)-2-amino-3-methyl-N-(2-morpholinoethyl)-pentanamide, or a pharmaceutically acceptable salt, solvate, ester, or prodrug thereof, in an approximately equal amount.

In one embodiment, the present invention provides a mixture of (2R,3S)-2-amino-3-methyl-N-(2-morpholinoethyl)-pentanamide and (2S,3R)-2-amino-3-methyl-N-(2-morpholinoethyl)-pentanamide, or a pharmaceutically acceptable salt, solvate, ester, or prodrug thereof. The individual compounds in the mixture can be in any ratio or weight percentage. In one specific embodiment, the mixture consists of (2R,3S)-2-amino-3-methyl-N-(2-morpholinoethyl)-pentanamide and (2S,3R)-2-amino-3-methyl-N-(2-morpholinoethyl)-pentanamide, or a pharmaceutically acceptable salt, solvate, ester, or prodrug thereof.

In another aspect, the present invention provides a pharmaceutical composition comprising the compound selected from the group consisting of (2R,3R)-2-amino-3-methyl-N-(2-morpholinoethyl)-pentanamide; (2R,3S)-2-amino-3-methyl-N-(2-morpholinoethyl)-pentanamide; and (2S,3R)-2-amino-3-methyl-N-(2-morpholinoethyl)-pentanamide, or a pharmaceutically acceptable salt, solvate, ester, or prodrug thereof; and a pharmaceutically acceptable carrier.

In another aspect, the present invention provides a pharmaceutical composition comprising a mixture of two or more compounds selected from the group consisting of (2S,3S)-2-amino-3-methyl-N-(2-morpholinoethyl)-pentanamide; (2R,3R)-2-amino-3-methyl-N-(2-morpholinoethyl)-pentanamide; (2R,3S)-2-amino-3-methyl-N-(2-morpholinoethyl)-pentanamide; and (2S,3R)-2-amino-3-methyl-N-(2-morpholinoethyl)-pentanamide; or a pharmaceutically acceptable salt, solvate, ester, or prodrug thereof; and a pharmaceutically acceptable carrier, with the proviso that when the mixture consists of (2S,3S)-2-amino-3-methyl-N-(2-morpholinoethyl)-pentanamide and (2R,3R)-2-amino-3-methyl-N-(2-morpholinoethyl)-pentanamide, or a pharmaceutically acceptable salt, solvate, ester, or prodrug thereof; then (2S,3S)-2-amino-3-methyl-N-(2-morpholinoethyl)-pentanamide, or a pharmaceutically acceptable salt, solvate, ester, or prodrug thereof, is in an amount not less than about 5% by weight.

The application provides compounds having binding specificity for a p75^{NTR} molecule. These compounds, along with related pharmaceutical compounds and methods, are useful in the treatment and prevention of neurodegenerative and other disorders.

A set of compounds disclosed herein are labeled as follows:

TABLE I
Structures of Compounds i-vii

| Compound | Name |
|---|---|
| 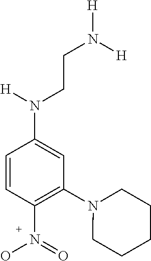 | Compound (i) (also referred to herein as “LM11A-28”) |
| 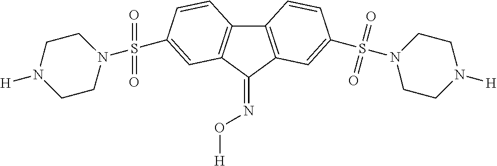 [SMILES: O=S(=O)(c1ccc2c(c1)C(=NO)c1cc(S(=O)(=O)N3CCNCC3)ccc1-2)N1CCNCC1] | Compound (ii) (also referred to herein as “LM11A-7”) |
| 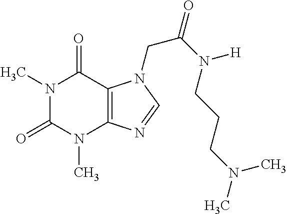 [SMILES: CN(C)CCCNC(=O)Cn1cnc2c1c(=O)n(C)c(=O)n2C] | Compound (iii) (also referred to herein as “LM11A-24”, “24”, and “C24”) |
| 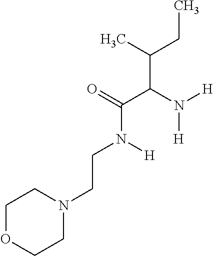 [SMILES: CCC(C)C(N)C(=O)NCCN1CCOCC1] | Compound (iv) (also referred to herein as “LM11A-31” and “31”) |
| 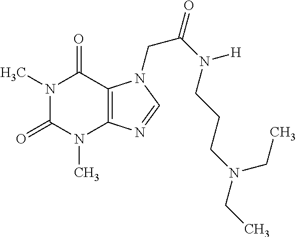 [SMILES: CCN(CC)CCCNC(=O)Cn1cnc2c1c(=O)n(C)c(=O)n2C] | Compound (v) (also referred to herein as “LM11A-36”, “36”, and “C36”) |
| 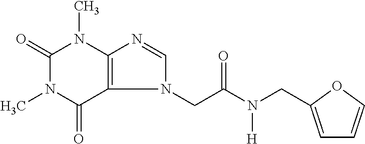 [SMILES: Cn1c(=O)c2c(ncn2CC(=O)NCc2ccco2)n(C)c1=O] | Compound (vi) (also referred to herein as “LM11A-38” and “C38”) |
| 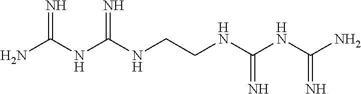 [SMILES: N=C(N)NC(=N)NCCNC(=N)NC(=N)N] | Compound (vii) |

One object of the presently disclosed subject matter is to provide methods of facilitating cell survival using neurotrophin mimetics.

In accordance with the presently disclosed subject matter, a representative neurotrophin can include, but is not limited to, NGF. More particularly, the neurotrophin β-turn loop having binding specificity for a p75^{NTR} molecule includes, but is not limited to, loop 1 of the NGF.

As disclosed herein, representative structures of the compound or mimetic having binding specificity for a p75^{NTR} molecule are capable of binding to the neurotrophin-binding site of the p75^{NTR} molecule.

The compounds disclosed herein can also encompass derivatives of a parent compound, which has binding specificity for a p75^{NTR} molecule, wherein the derivative also has binding specificity for the p75^{NTR}. The derivative can exhibit enhancement in at least one of the characteristics selected from the group consisting of hydrophilicity, lipophilicity, amphipathicity, solubility, bioavailability, and resistance to hepatic degradation, as compared to the parent compound.

It is to be understood that in some embodiments the compounds disclosed herein can encompass a pharmacophore substantially identical to the pharmacophore illustrated in FIG. 1c. Representative such compounds include but are not limited to compounds encompassed by Formulas (A) and (B).

The aforementioned individual compounds or mixtures can be used for treating a wide range of conditions and diseases described herein.

In one aspect, there is provided a pharmaceutical composition comprising a pharmaceutically acceptable diluent or carrier and a compound of Formula I, IA, IB, II, IIA, IIB, III, IV or WA or a pharmaceutically acceptable salt, ester, prodrug or solvate thereof.

In another aspect, there is provided a method for treating a disorder associated with p75 expression comprising administering to a patient in need of such treatment a compound of Formula I, IA, IB, II, IIA, IIB, III, IV or IVA or a pharmaceutically acceptable salt, ester, solvate or prodrug thereof. In one embodiment the disorder is associated directly with cells expressing p75; in another embodiment, the cells do not express p75, but are affected by p75 expression.

In another aspect, there is provided a method for activating a p75 receptor comprising contacting a cell containing a p75 receptor with a compound of Formula I, IA, IB, II, IIA, IIB, III, IV or IVA or a pharmaceutically acceptable salt, ester, solvate or prodrug thereof.

In another aspect, there is provided a method for the treatment of disorders involving degeneration or dysfunction of cells expressing p75 comprising administering to a patient in need of such treatment a compound of Formula I, IA, IB, II, IIA, IIB, III, IV or IVA or a pharmaceutically acceptable salt, ester, solvate or prodrug thereof. In one embodiment, the method of treatment comprises facilitating cell survival; in another embodiment, the method of treatment comprises inhibiting degenerative p75 signaling; in yet another embodiment, the method of treatment comprises inhibiting dysfunctional p75 signaling. In one embodiment, the disorder is a neurodegenerative disorder. In another embodiment, the disorder is selected from the group consisting of Alzheimer's disease, Huntington's disease, Pick's disease, amyotrophic lateral sclerosis, epilepsy, Parkinson's disease, spinal cord injury, stroke, hypoxia, ischemia, brain injury, diabetic neuropathy, peripheral neuropathy, nerve transplantation, multiple sclerosis, peripheral nerve injury and hair loss.

Compounds of Formula I, IA, IB, II, IIA, IIB, III, IV or IVA or a pharmaceutically acceptable salt, ester, solvate or prodrug thereof as disclosed herein that target p75 receptors expressed by neurons are used to prevent loss of function, degeneration and/or death of neurons in a number of nervous system disorders. Such disorders include, but are not limited to, Alzheimer's disease, Parkinson's disease, Huntington's disease, stroke, traumatic brain injury, spinal cord injury, epilepsy, multiple sclerosis, amyotrophic lateral sclerosis, neuropathies, myopathies and various forms of retinal degeneration. In one embodiment, compounds of the present application are used in the treatment of Alzheimer's disease. Compounds of Formula I, IA, IB, II, IIA, IIB, III, IV or IVA or a pharmaceutically acceptable salt, ester, solvate or prodrug thereof as disclosed herein that target p75 receptors expressed by oligodendrocytes are used to prevent loss of function, degeneration and/or death of oligodendrocytes in a number of nervous system disorders including, but not limited to, multiple sclerosis, spinal cord injury and perinatal anoxia. In another embodiment, compounds of the present application are used to treat multiple sclerosis.

Outside of the nervous system, a number of cell populations express the p75 receptor. These include hair follicle cells, hepatic cells, vascular endothelial, vascular smooth muscle cells, cardiomyocytes. In addition, the p75 receptor is expressed by certain tumor cells such as those involved in breast or prostate cancer. Given this expression pattern, compounds of Formula I, IA, IB, II, IIA, IIB, III, IV or IVA or a pharmaceutically acceptable salt, ester, solvate or prodrug thereof as disclosed herein that target p75 receptors are used to: prevent loss of hair follicle cells and thereby prevent hair loss; prevent hepatic cirrhosis and promote liver regeneration; regulate angiogenesis and promote neovascularization in the setting of diabetic wounds or other ischemic settings; prevent cardiomyopathy e.g. preventing myocardial cell loss or stimulating growth of new cardiomyocytes either in the setting of ischemia or after myocardial infarction; and inhibit tumor cell growth. In addition p75 is expressed by stem cells and is known to regulate stem cell growth; therefore, p75 ligands are used to promote stem cell growth as part of a strategy to promote tissue and organ regeneration.

In one variation of any of the disclosed aspects or embodiments, the compound administered to a patient in need thereof is selected from the group consisting of:

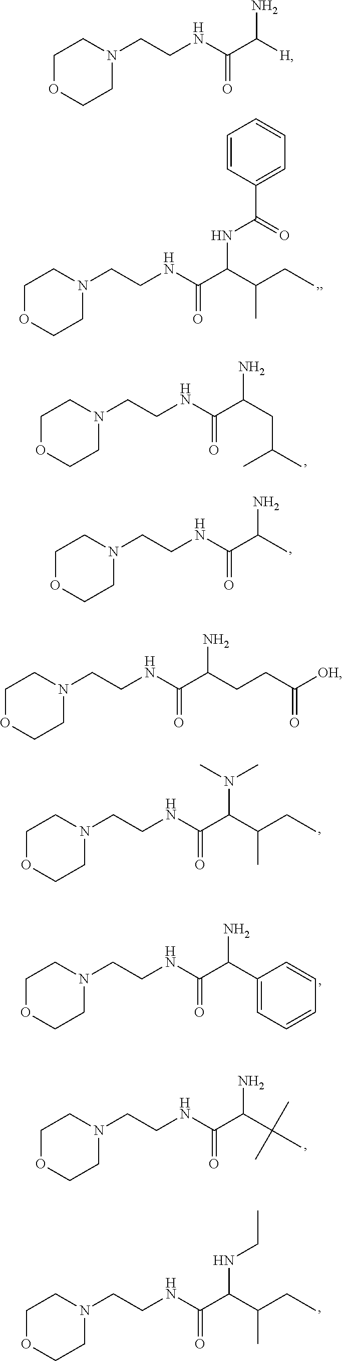

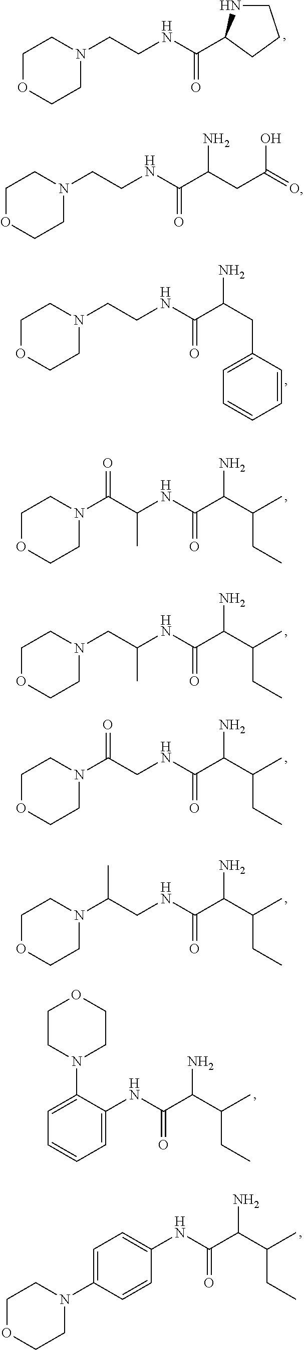

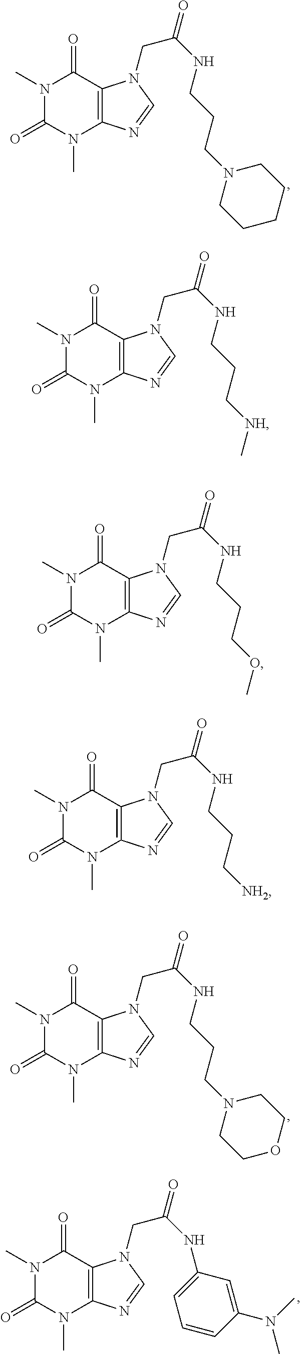

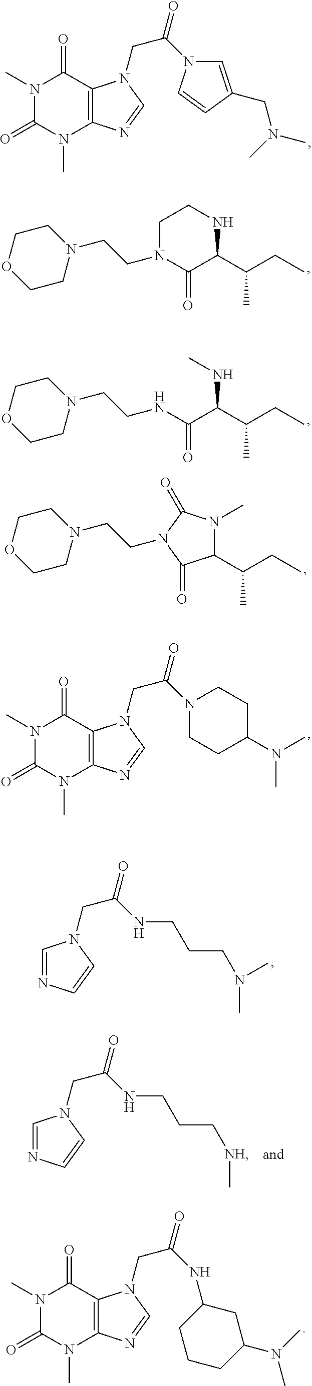

In another variation of any of the disclosed embodiments or aspects, the compound administered to a patient in need thereof is selected from the group consisting of:

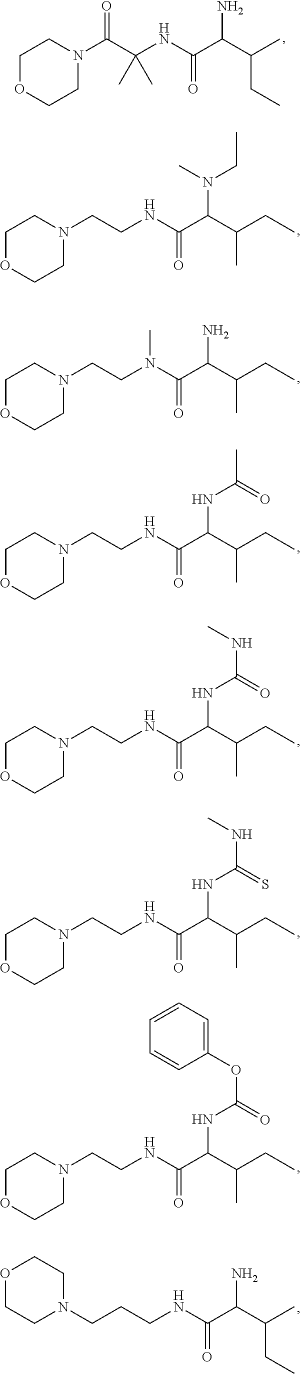

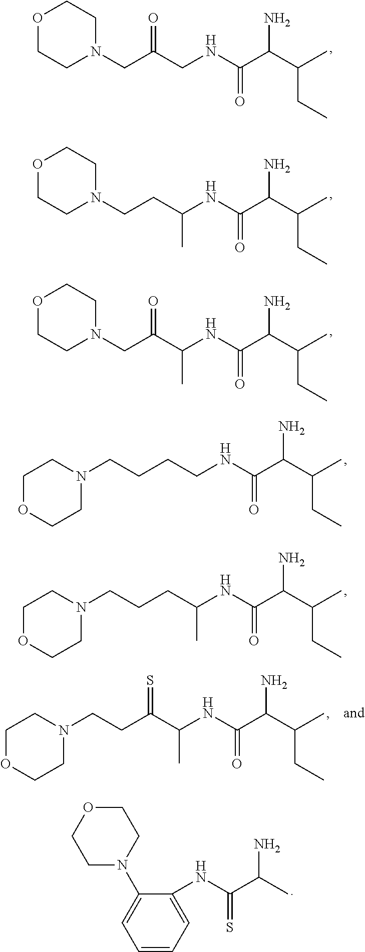

In another aspect, there is provided a method for treating a disorder associated with p75 expression comprising administering to a patient in need of such treatment a stereoisomer of 2 amino-3-methyl-N-(2-morpholinoethyl)-pentanamide, including (2R,3R)-2-amino-3-methyl-N-(2-morpholinoethyl)-pentanamide; (2R,3S)-2-amino-3-methyl-N-(2-morpholinoethyl)-pentanamide; and (2S,3R)-2-amino-3-methyl-N-(2-morpholinoethyl)-pentanamide or a pharmaceutically acceptable salt, ester, solvate or prodrug thereof. In one embodiment the disorder is associated directly with cells expressing p75; in another embodiment, the cells do not express p75, but are affected by p75 expression. In another aspect, there is provided a method for activating a p75 receptor comprising contacting a cell containing a p75 receptor with a stereoisomer of 2 amino-3-methyl-N-(2-morpholinoethyl)-pentanamide, including (2R,3R)-2-amino-3-methyl-N-(2-morpholinoethyl)-pentanamide; (2R,3S)-2-amino-3-methyl-N-(2-morpholinoethyl)-pentanamide; and (2S,3R)-2-amino-3-methyl-N-(2-morpholinoethyl)-pentanamide or a pharmaceutically acceptable salt, ester, solvate or prodrug thereof.

In another aspect, there is provided a method for the treatment of disorders involving degeneration or dysfunction of cells expressing p75 comprising administering to a patient in need of such treatment a stereoisomer of 2 amino-3-methyl-N-(2-morpholinoethyl)-pentanamide, including (2R,3R)-2-amino-3-methyl-N-(2-morpholinoethyl)-pentanamide; (2R,3S)-2-amino-3-methyl-N-(2-morpholinoethyl)-pentanamide; and (2S,3R)-2-amino-3-methyl-N-(2-morpholinoethyl)-pentanamide or a pharmaceutically acceptable salt, ester, solvate or prodrug thereof. In one embodiment, the method of treatment comprises facilitating cell survival; in another embodiment, the method of treatment comprises inhibiting degenerative p75 signalling; in yet another embodiment, the method of treatment comprises inhibiting dysfunctional p75 signaling. In one embodiment, the disorder is a neurodegenerative disorder. In another embodiment, the disorder is selected from the group consisting of Alzheimer's disease, Huntington's disease, Pick's disease, amyotrophic lateral sclerosis, epilepsy, Parkinson's disease, spinal cord injury, stroke, hypoxia, ischemia, brain injury, diabetic neuropathy, peripheral neuropathy, nerve transplantation, multiple sclerosis, peripheral nerve injury and hair loss.

Stereoisomers of 2 amino-3-methyl-N-(2-morpholinoethyl)-pentanamide, including (2R,3R)-2-amino-3-methyl-N-(2-morpholinoethyl)-pentanamide; (2R,3S)-2-amino-3-methyl-N-(2-morpholinoethyl)-pentanamide; and (2S,3R)-2-amino-3-methyl-N-(2-morpholinoethyl)-pentanamide or a pharmaceutically acceptable salt, ester, solvate or prodrug thereof as disclosed herein that target p75 receptors expressed by neurons are used to prevent loss of function, degeneration and/or death of neurons in a number of nervous system disorders. Such disorders include, but are not limited to, Alzheimer's disease, Parkinson's disease, Huntington's disease, stroke, traumatic brain injury, spinal cord injury, epilepsy, multiple sclerosis, amyotrophic lateral sclerosis, neuropathies, myopathies and various forms of retinal degeneration. In one embodiment, compounds of the present application are used in the treatment of Alzheimer's disease. Stereoisomers of 2 amino-3-methyl-N-(2-morpholinoethyl)-pentanamide, including (2R,3R)-2-amino-3-methyl-N-(2-morpholinoethyl)-pentanamide; (2R,3S)-2-amino-3-methyl-N-(2-morpholinoethyl)-pentanamide; and (2S,3R)-2-amino-3-methyl-N-(2-morpholinoethyl)-pentanamide or a pharmaceutically acceptable salt, ester, solvate or prodrug thereof as disclosed herein that target p75 receptors expressed by oligodendrocytes are used to prevent loss of function, degeneration and/or death of oligodendrocytes in a number of nervous system disorders including, but not limited to, multiple sclerosis, spinal cord injury and perinatal anoxia. In another embodiment, compounds of the present application are used to treat multiple sclerosis.

Outside of the nervous system, a number of cell populations express the p75 receptor. These include hair follicle cells, hepatic cells, vascular endothelial, vascular smooth muscle cells, cardiomyocytes. In addition, the p75 receptor is expressed by certain tumor cells such as those involved in breast or prostate cancer. Given this expression pattern, stereoisomers of 2 amino-3-methyl-N-(2-morpholinoethyl)-pentanamide, including (2R,3R)-2-amino-3-methyl-N-(2-morpholinoethyl)-pentanamide; (2R,3S)-2-amino-3-methyl-N-(2-morpholinoethyl)-pentanamide; and (2S,3R)-2-amino-3-methyl-N-(2-morpholinoethyl)-pentanamide or a pharmaceutically acceptable salt, ester, solvate or prodrug thereof as disclosed herein that target p75 receptors are used to: prevent loss of hair follicle cells and thereby prevent hair loss; prevent hepatic cirrhosis and promote liver regeneration; regulate angiogenesis and promote neovascularization in the setting of diabetic wounds or other ischemic settings; prevent cardiomyopathy e.g. preventing myocardial cell loss or by stimulating growth of new cardiomyocytes either in the setting of ischemia or after myocardial infarction; and inhibit tumor cell growth. In addition p75 is expressed by stem cells and is known to regulate stem cell growth; therefore, p75 ligands are used to promote stem cell growth as part of a strategy to promote tissue and organ regeneration.

In one variation of any disclosed aspect or embodiment, the method comprises administering to a patient in need of such treatment a pharmaceutical composition comprising a mixture of two or more compounds selected from the group consisting of (2S,3S)-2-amino-3-methyl-N-(2-morpholinoethyl)-pentanamide; (2R,3R)-2-amino-3-methyl-N-(2-morpholinoethyl)-pentanamide; (2R,3S)-2-amino-3-methyl-N-(2-morpholinoethyl)-pentanamide; and (2S,3R)-2-amino-3-methyl-N-(2-morpholinoethyl)-pentanamide; or a pharmaceutically acceptable salt, solvate, ester, or prodrug thereof, with the proviso that when the mixture consists of (2S,3S)-2-amino-3-methyl-N-(2-morpholinoethyl)-pentanamide and (2R,3R)-2-amino-3-methyl-N-(2-morpholinoethyl)-pentanamide, or a pharmaceutically acceptable salt, solvate, ester, or prodrug thereof; then (2S,3S)-2-amino-3-methyl-N-(2-morpholinoethyl)-pentanamide, or a pharmaceutically acceptable salt, solvate, ester, or prodrug thereof, is in an amount not less than about 5% by weight based on the total amount of the mixture. In another variation of any disclosed aspect or embodiment, the method comprises administering to a patient in need of such treatment a pharmaceutical composition comprising a mixture of (2R,3R)-2-amino-3-methyl-N-(2-morpholinoethyl)-pentanamide and (2S,3S)-2-amino-3-methyl-N-(2-morpholinoethyl)-pentanamide, or a pharmaceutically acceptable salt, solvate, ester, or prodrug thereof, with the proviso that (2S,3S)-2-amino-3-methyl-N-(2-morpholinoethyl)-pentanamide, or a pharmaceutically acceptable salt, solvate, ester, or prodrug thereof, is in an amount not less than about 5% by weight based on the total amount of the mixture. In yet another variation of any disclosed aspect or embodiment, the method comprises administering, to a patient in need of such treatment a pharmaceutical composition comprising a mixture of (2R,3S)-2-amino-3-methyl-N-(2-morpholinoethyl)-pentanamide and (2S,3R)-2-amino-3-methyl-N-(2-morpholinoethyl)-pentanamide, or a pharmaceutically acceptable salt, solvate, ester, or prodrug thereof.

### FORMULATIONS

For the purposes of this invention, the compounds may be administered by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

The compounds disclosed herein can be formulated in accordance with the routine procedures adapted for desired administration route. Accordingly, the compounds disclosed herein can take such forms as suspensions, solutions or emulsions in oily or aqueous vehicles, and can contain formulatory agents such as suspending, stabilizing and/or dispersing agents. The compounds disclosed herein can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). Alternatively, the active ingredient can be in powder form for constitution with a suitable vehicle, e.g., sterile pyrogen-free water, before use. Suitable formulations for each of these methods of administration can be found, for example, in Remington: The Science and Practice of Pharmacy, A. Gennaro, ed., 20th edition, Lippincott, Williams & Wilkins, Philadelphia, Pa.

For example, formulations for parenteral administration can contain as common excipients sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers can be useful excipients to control the release of active compounds. Other potentially useful parenteral delivery systems include ethylene-vinyl acetate copolymer particles, osmotic pumps, implantable infusion systems, and liposomes. Formulations for inhalation administration contain as excipients, for example, lactose, or can be aqueous solutions containing, for example, polyoxyethylene-9-auryl ether, glycocholate and deoxycholate, or oily solutions for administration in the form of nasal drops, or as a gel to be applied intranasally. Formulations for parenteral administration can also include glycocholate for buccal administration, methoxysalicylate for rectal administration, or citric acid for vaginal administration.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables. Formulations for intravenous administration can comprise solutions in sterile isotonic aqueous buffer. Where necessary, the formulations can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where the compound is to be administered by infusion, it can be dispensed in a formulation with an infusion bottle containing sterile pharmaceutical grade water, saline or dextrose/water. Where the compound is administered by injection, an ampule of sterile water for injection or saline can be provided so that the ingredients can be mixed prior to administration.

Suitable formulations further include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

The compounds can further be formulated for topical administration. Suitable topical formulations include one or more compounds in the form of a liquid, lotion, cream or gel. Topical administration can be accomplished by application directly on the treatment area. For example, such application can be accomplished by rubbing the formulation (such as a lotion or gel) onto the skin of the treatment area, or by spray application of a liquid formulation onto the treatment area.

In some formulations, bioimplant materials can be coated with the compounds so as to improve interaction between cells and the implant.

Formulations of the compounds can contain minor amounts of wetting or emulsifying agents, or pH buffering agents. The formulations comprising the compound can be a liquid solution, suspension, emulsion, tablet, pill, capsule, sustained release formulation, or powder.

The compounds can be formulated as a suppository, with traditional binders and carriers such as triglycerides.

Pharmaceutical compositions containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Oral formulations can include standard carriers such as pharmaceutical grades of mannitol, lactose, starch, magnesium stearate, polyvinyl pyrrolidone, sodium saccharine, cellulose, magnesium carbonate, etc. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax maybe employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

The pharmaceutical formulations comprising the compounds of the present application can include an agent which controls release of the compound, thereby providing a timed or sustained release compound.

### Carriers

Pharmaceutically acceptable carriers are well known to those skilled in the art and include, but are not limited to, from about 0.01 to about 0.1 M and preferably 0.05M phosphate buffer or 0.8% saline. Such pharmaceutically acceptable carriers can be aqueous or non-aqueous solutions, suspensions and emulsions.

Examples of non-aqueous solvents suitable for use in the present application include, but are not limited to, propylene glycol, polyethylene glycol, vegetable oils such as olive oil, and injectable organic esters such as ethyl oleate.

Aqueous carriers suitable for use in the present application include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the present application can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the present application include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellent.

Solid carriers suitable for use in the present application include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

Parenteral carriers suitable for use in the present application include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the present application can be mixed as needed with disintegrants, diluents, granulating agents, lubricants, binders and the like using conventional techniques known in the art. The carriers can also be sterilized using methods that do not deleteriously react with the compounds, as is generally known in the art.

### Salts

It is also to be understood that the disclosed compounds can further comprise pharmaceutically acceptable salts.

Such salts include, but are not limited to, pharmaceutically acceptable acid addition salts, pharmaceutically acceptable base addition salts, pharmaceutically acceptable metal salts, ammonium and alkylated ammonium salts.

Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like.

Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine dicyclohexylamine and the like.

Examples of metal salts include lithium, sodium, potassium, magnesium salts and the like.

Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like.

Standard methods for the preparation of pharmaceutically acceptable salts and their formulations are well known in the art, and are disclosed in various references, including for example, “Remington: The Science and Practice of Pharmacy”, A. Gennaro, ed., 20th edition, Lippincott, Williams & Wilkins, Philadelphia, Pa.

### Methods of Use

As disclosed throughout, the present application provides treatment of disorders associated with p75 expression. Generally, the present application provides treatment of disorders involving degradation or dysfunction of cells expressing p75.

In one aspect, there is provided a method for activating p75 receptors comprising contacting a cell containing a p75 receptor with one or more compounds of the present application or a pharmaceutically acceptable salt, ester, solvate or prodrug thereof. Additionally disclosed are methods for treating nervous system disorders including, but not limited to, Alzheimer's disease, Parkinson's disease, Huntington's disease, stroke, traumatic brain injury, spinal cord injury, epilepsy, multiple sclerosis, amyotrophic lateral sclerosis, neuropathies, myopathies and various forms of retinal degeneration, based on the ability of the compounds of the present application to target p75 receptors expressed by neurons or other cells.

Further disclosed are methods for treating nervous system disorders including, but not limited to, multiple sclerosis, spinal cord injury and perinatal anoxia, based on the ability of the compounds of the present application to target p75 receptors expressed by oligodendrocytes, microglia or astrocytes.

Additionally disclosed are methods for treating diseases other than those of the nervous system, including methods to: prevent loss of hair follicle cells and thereby prevent hair loss; prevent hepatic cirrhosis and promote liver regeneration; regulate angiogenesis and promote neovascularization in the setting of diabetic wounds or other ischemic settings; prevent cardiomyopathy e.g. preventing myocardial cell loss or stimulating growth of new cardiomyocytes either in the setting of ischemic or after myocardial infarction; and inhibit tumor cell growth. In addition p75 is expressed by stem cells and is known to regulate stem cell growth; therefore, p75 ligands disclosed herein can be used to promote stem cell growth as part of a strategy to promote tissue and organ regeneration.

The present application also provides methods of treating neurodegenerative and other disorders or conditions in a subject. Generally, the methods of the present application involve administration of a compound having binding specificity for a p75^{NTR} molecule in a subject to treat a neurodegenerative disorder or other disorder or condition. The compound can be administered in an amount effective to induce survival signaling and/or inhibit proNGF-induced cell death, which has been determined to be associated with neurodegenerative and other disorders.

The condition to be treated can be any condition which is mediated, at least in part, by binding of neurotrophins to p75^{NTR}. Such conditions include, but are not limited to, Alzheimer's disease, Huntington's disease, Pick's disease, amyotrophic lateral sclerosis, epilepsy, Parkinson's disease, spinal cord injury, stroke, hypoxia, ischemia, brain injury, diabetic neuropathy, peripheral neuropathy, nerve transplantation, multiple sclerosis, peripheral nerve injury, and hair loss.

In general, the condition to be treated can be any condition which is mediated, at least in part, by aberrant signaling of the p75 receptor. In one embodiment, the aberrant signaling is mediated by the presence or absence of neurotrophin binding; in another embodiment the aberrant signaling is not mediated by the presence or absence of neurotrophin binding. In one variation aberrant signaling occurs in the absence of neurotrophin binding.

Compounds having binding specificity for p75^{NTR} as disclosed herein can be used to treat cell degeneration, including preventing neurodegeneration such as, for example, neurodegeneration caused by chemotherapy and/or neurodegenerative disorders, as well as other conditions such as inducing hair follicle cell survival after hair follicle cell degeneration caused by, for example, chemotherapy.

The present application further provides for novel methods of facilitating cell survival. Representative cells include, but are not limited to, septal, hippocampal, cortical, sensory, sympathetic, motor neurons, hair follicle cells, progenitor, and stem cells. Generally, such cells include neurons, oligodendrocytes and hair follicle cells. Specifically, the methods comprise treating a cell with a compound having binding specificity for a p75^{NTR} molecule, whereby the compound induces survival signaling and inhibits proNGF-induced cell death.

The present application also provides for a novel method of optimizing cell function comprising use of the compounds disclosed herein. In one embodiment, decreased cell survival is not the primary underlying disease mechanism; in another embodiment, decreased cell survival is the primary underlying disease mechanism.

### Administration

The present application discloses a method of administering compounds having binding specificity for p75^{NTR} in order to ameliorate a condition mediated by p75^{NTR} binding in a subject. The method can comprise the step of administering to a subject an effective amount of a compound having binding specificity for p75^{NTR}, such as any of the compounds disclosed herein.

As used herein, administering can be effected or performed using any of the various methods known to those skilled in the art. The compound can be administered, for example, subcutaneously, intravenously, parenterally, intraperitoneally, intradermally, intramuscularly, topically, enteral (e.g., orally), rectally, nasally, buccally, sublingually, vaginally, by inhalation spray, by drug pump or via an implanted reservoir in dosage formulations containing conventional non-toxic, physiologically acceptable carriers or vehicles.

Further, the presently disclosed compounds can be administered to a localized area in need of treatment. This can be achieved by, for example, and not by way of limitation, local infusion during surgery, topical application, transdermal patches, by injection, by catheter, by suppository, or by implant (the implant can optionally be of a porous, non-porous, or gelatinous material), including membranes, such as sialastic membranes or fibers.

The form in which the compound is administered (e.g., syrup, elixir, capsule, tablet, solution, foams, emulsion, gel, sol) will depend in part on the route by which it is administered. For example, for mucosal (e.g., oral mucosa, rectal, intestinal mucosa, bronchial mucosa) administration, nose drops, aerosols, inhalants, nebulizers, eye drops or suppositories can be used. The compound can also be used to coat bioimplantable materials to enhance neurite outgrowth, neural survival, or cellular interaction with the implant surface. The compounds and agents disclosed herein can be administered together with other biologically active agents, such as analgesics, anti-inflammatory agents, anesthetics and other agents which can control one or more symptoms or causes of a p75^{NTR}-mediated condition.

Additionally, administration can comprise administering to the subject a plurality of dosages over a suitable period of time. Such administration regimens can be determined according to routine methods, upon a review of the instant disclosure.

The compounds of the present application can be employed as the sole active agent in a pharmaceutical or can be used in combination (e.g., administered proximate in time to each other or even in the same formulation) with other active ingredients, e.g., neurotrophins, or other factors or drugs which can facilitate neural survival or axonal growth in neurodegenerative diseases, including but not limited to amyloid-β inhibitors, acetylcholinesterase inhibitors, butyrylcholinesterase inhibitors, and N-methyl-D-aspartate subtype of glutamate receptor antagonists.

### Dosage

Compounds of the invention are generally administered in a dose of about 0.01 mg/kg/dose to about 100 mg/kg/dose. Alternately the dose can be from about 0.1 mg/kg/dose to about 10 mg/kg/dose; or about 1 mg/kg/dose to 10 mg/kg/dose. In some dosages, the compounds disclosed herein are administered at about 5 mg/kg/dose. Time release preparations may be employed or the dose may be administered in as many divided doses as is convenient. When other methods are used (e.g. intravenous administration), compounds are administered to the affected tissue at a rate from about 0.05 to about 10 mg/kg/hour, alternately from about 0.1 to about 1 mg/kg/hour. Such rates are easily maintained when these compounds are intravenously administered as discussed herein. Generally, topically administered formulations are administered in a dose of about 0.5 mg/kg/dose to about 10 mg/kg/dose range. Alternately, topical formulations are administered at a dose of about 1 mg/kg/dose to about 7.5 mg/kg/dose or even about 1 mg/kg/dose to about 5 mg/kg/dose.

A range of from about 0.1 to about 100 mg/kg is appropriate for a single dose. Continuous administration is appropriate in the range of about 0.05 to about 10 mg/kg. Topical administration is appropriate for conditions such as hair loss or wound revascularization.

Drug doses can also be given in milligrams per square meter of body surface area rather than body weight, as this method achieves a good correlation to certain metabolic and excretionary functions. Moreover, body surface area can be used as a common denominator for drug dosage in adults and children as well as in different animal species (Freireich et al., (1966) Cancer Chemother Rep. 50, 219-244). Briefly, to express a mg/kg dose in any given species as the equivalent mg/sq in dose, the dosage is multiplied by the appropriate km factor. In an adult human, 100 mg/kg is equivalent to 100 mg/kg×37 kg/sq m=3700 mg/m².

Insofar as the compounds disclosed herein can take the form of a mimetic or fragment thereof, it is to be appreciated that the potency, and therefore dosage of an effective amount can vary. However, one skilled in the art can readily assess the potency of a compound of the type presently envisioned by the present application.

In settings of a gradually progressive nervous system disorder, compounds of the present application are generally administered on an ongoing basis. In certain settings administration of a compound disclosed herein can commence prior to the development of disease symptoms as part of a strategy to delay or prevent the disease. In other settings a compound disclosed herein is administered after the onset of disease symptoms as part of a strategy to slow or reverse the disease process and/or part of a strategy to improve cellular function and reduce symptoms. Compounds have been developed that cross the blood brain barrier and hence would be delivered by oral administration or by other peripheral routes. Compounds that do not cross the blood brain barrier are applied for targets outside of the central nervous system. For targets and tissues outside of the nervous system, compounds are applied in either acute or chronic settings by other oral or directed target administration such as by topical application.

It will be appreciated by one of skill in the art that dosage range will depend on the particular compound, and its potency. The dosage range is understood to be large enough to produce the desired effect in which the neurodegenerative or other disorder and the symptoms associated therewith are ameliorated and/or survival of the cells is achieved, but not be so large as to cause unmanageable adverse side effects. It will be understood, however, that the specific dose level for any particular patient will depend on a variety of factors including the activity of the specific compound employed; the age, body weight, general health, sex and diet of the individual being treated; the time and route of administration; the rate of excretion; other drugs which have previously been administered; and the severity of the particular disease undergoing therapy, as is well understood by those skilled in the art. The dosage can also be adjusted by the individual physician in the event of any complication. No unacceptable toxicological effects are expected when compounds disclosed herein are used in accordance with the present application.

An effective amount of the compounds disclosed herein comprise amounts sufficient to produce a measurable biological response. Actual dosage levels of active ingredients in a therapeutic compound of the present application can be varied so as to administer an amount of the active compound that is effective to achieve the desired therapeutic response for a particular subject and/or application. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

Further with respect to the methods of the present application, a preferred subject is a vertebrate subject. A preferred vertebrate is warm-blooded; a preferred warm-blooded vertebrate is a mammal. The subject treated by the presently disclosed methods is desirably a human, although it is to be understood that the principles of the present application indicate effectiveness with respect to all vertebrate species which are to included in the term “subject.” In this context, a vertebrate is understood to be any vertebrate species in which treatment of a neurodegenerative disorder is desirable. As used herein, the term “subject” includes both human and animal subjects. Thus, veterinary therapeutic uses are provided in accordance with the present application.

As such, the present application provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economical importance to humans. Thus, also provided is the treatment of livestock, including, but not limited to, domesticated swine, ruminants, ungulates, horses (including race horses), poultry, and the like.

### EXAMPLES

### General Syntheses

Standard procedures and chemical transformation and related methods are well known to one skilled in the art, and such methods and procedures have been described, for example, in standard references such as Fiesers' Reagents for Organic Synthesis, John Wiley and Sons, New York, N.Y., 2002; Organic Reactions, vols. 1-83, John Wiley and Sons, New York, N.Y., 2006; March J. and Smith M., Advanced Organic Chemistry, 6th ed., John Wiley and Sons, New York, N.Y.; and Larock R. C., Comprehensive Organic Transformations, Wiley-VCH Publishers, New York, 1999. All texts and references cited herein are incorporated by reference in their entirety.

Reactions using compounds having functional groups may be performed on compounds with functional groups that may be protected. A “protected” compound or derivatives means derivatives of a compound where one or more reactive site or sites or functional groups are blocked with protecting groups. Protected derivatives are useful in the preparation of the compounds of the present invention or in themselves; the protected derivatives may be the biologically active agent. An example of a comprehensive text listing suitable protecting groups may be found in T. W. Greene, Protecting Groups in Organic Synthesis, 3rd edition, John Wiley & Sons, Inc. 1999.

Preparation of many of the compounds, e.g. Compounds 1-21, can be illustrated in the general Scheme 1 below:

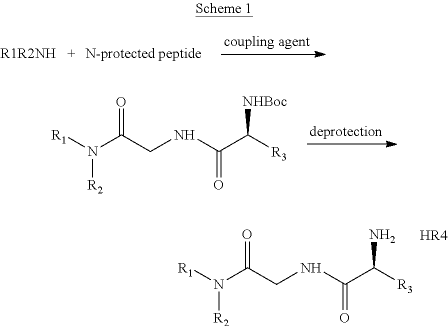

Generally the protection group for the amino acid is a Boc group. The coupling agent can be HATU, HBTU, EDC/HOBt, or DCC/DMAP. The deprotection reagent can be 4 M HCl in MeOH, 4M HCl in water, or TFA in DCM.

Generally, an amine or aniline is coupled with an N-protected amino acid and this coupled intermediate is deprotected to give a final compound or another intermediate. The second intermediate can be further modified or directly go through this coupling-deprotection cycle one more time to give the final compound.

### Example 1

### Preparation of Compound 1

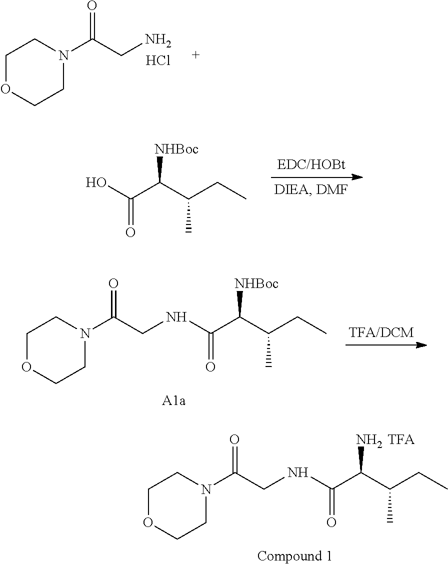 [SMILES: NCC(=O)N1CCOCC1 ; Cl ; CC[C@H](C)[C@H](NC(=O)OC(C)(C)C)C(=O)O ; CC[C@H](C)[C@H](NC(=O)OC(C)(C)C)C(=O)NCC(=O)N1CCOCC1 ; CC[C@H](C)[C@H](N)C(=O)NCC(=O)N1CCOCC1]

Preparation of Intermediate A1a

| S. | | | | | |
|---|---|---|---|---|---|
| No. | Chemicals/Reagents & Solvent | MW | mmol | Eq. | Amts |
| 1 | Boc-Ile-OH | 231.29 | 1 | 1.0 | 231 mg |
| 2 | DCM (anhydrous) | | | | 5 mL |
| 3 | DIEA, d = 0.742 | 129.25 | 2.5 | 2.5 | 0.44 mL |
| 4 | EDC | 191.7 | 1 | 1.0 | 192 mg |
| 5 | HOBt | 135.13 | 1 | 1.0 | 135 mg |
| 6 | Amine | 180.63 | 1 | 1.0 | 180 mg |

To a solution of Boc-Ile-OH in DCM were added DIEA, HOBt, EDC, and amine in that order. The reaction mixture was stirred for 3 hours at room temperature (RT).

After the reaction was complete (LC-MS), 10% citric acid was added to quench the reaction. The DCM layer was separated and washed with saturated NaHCO₃, brine, dried with anhydrous Na₂SO₄, filtered, and concentrated. The intermediate Ala was obtained as an off-white solid (303 mg).

### Preparation of Compound 1

| S. No. | Chemicals/Reagents & Solvent | MW | mmol | Eq. | Amts |
|---|---|---|---|---|---|
| 1 | A1a | | | | 300 mg |
| 2 | TFA/DCM (1:1) | | | | 4 mL |

Cold TFA/DCM was added to the residue of A1a. The reaction mixture was stirred for 2 hours at room temperature. After the reaction was complete (LC-MS), the mixture was concentrated to afford the final compound (272 mg). Compound 1 was characterized by ¹H NMR, LC-MS and HPLC. ¹H NMR (MeOD), δ: 4.07-4.21 (m, 2H), 3.77 (d, J=5.84 Hz, 1H), 3.65-3.70 (m, 4H), 3.56-3.58 (m, 2H), 3.45-3.51 (m, 2H), 1.88-1.98 (m, 1H), 1.56-1.66 (m, 1H), 1.21-1.31 (m, 1H), 1.05 (d, J=6.78 Hz, 3H), 0.99 (t, J=7.40 Hz, 3H). LC-MS, (M+1), 258. HPLC (>95%, retention time, 1.99 min)

### Example 2

### Preparation of Compound 2

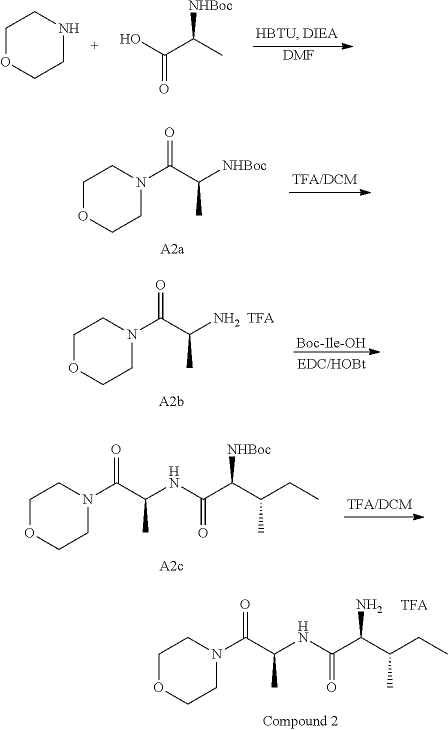

### Preparation of Intermediate A2a

| S. | | | | | |
|---|---|---|---|---|---|
| No. | Chemicals/Reagents & Solvents | MW | mmol | Eq. | Amts |
| 1 | Boc-Ala-OH | 189.21 | 2 | 1.0 | 378 mg |
| 2 | DMF (anhydrous) | | | | 5 mL |
| 3 | DIEA, d = 0.742 | 129.25 | 5 | 2.5 | 0.87 mL |
| 4 | HBTU | 379 | 2 | 1.0 | 758 mg |
| 5 | Morpholine, d = 0.99 | 87.12 | 2 | 1.0 | 175 uL |

To a solution of Boc-Ala-OH in DMF were added HBTU, DIEA, and morpholine in that order. The reaction mixture was stirred for 2 hours at room temperature. After the reaction was complete (LC-MS), the reaction mixture was subject to prep-HPLC purification to afford the intermediate A2a (333 mg).

### Preparation of Intermediate A2b

| S. No. | Chemicals/Reagents & Solvents | MW | mmol | Eq. | Amts |
|---|---|---|---|---|---|
| 1 | A2a | | | | 158 mg |
| 2 | TFA/DCM (1:1) | | | | 4 mL |

Cold TFA/DCM was added to the residue of A2a. The reaction mixture was stirred for 2 hours at room temperature. After the reaction was complete (LC-MS), the mixture was concentrated to afford the intermediate A2b (180 mg).

### Preparation of Intermediate A2c

| S. | | | | | |
|---|---|---|---|---|---|
| No. | Chemicals/Reagents & Solvents | MW | mmol | Eq. | Amts |
| 1 | Boc-Ile-OH | 231.29 | 0.611 | 1.0 | 141 mg |
| 2 | DCM (anhydrous) | | | | 5 mL |
| 3 | DIEA, d = 0.742 | 129.25 | 1.83 | 3.0 | 0.32 mL |
| 4 | HOBt•H₂O | 135.13 | 0.611 | 1.0 | 83 mg |
| 5 | EDC | 191.7 | 0.611 | 1.0 | 117 mg |
| 6 | A2b | | 0.611 | 1.0 | 180 mg |

To the residue of A2b were added DCM, Boc-Ala-OH, HOBt, EDC, and DIEA in that order. The reaction mixture was stirred for 2 hours at room temperature. After the reaction was complete (LC-MS), 0.5HCl was added to quench the reaction. The DCM layer was separated and washed with saturated NaHCO₃, brine, dried with anhydrous Na₂SO₄ and filtered. The liquid was concentrated to afford the intermediate A2c (64 mg).

### Preparation of Compound 2

| S. No. | Chemicals/Reagents & Solvents | MW | mmol | Eq. | Amts |
|---|---|---|---|---|---|
| 1 | A2c | | | | 60 mg |
| 2 | TFA/DCM (1:1) | | | | 4 mL |

Cold TFA/DCM was added to the residue of A2c. The reaction mixture was stirred for 3 hours at room temperature. After the reaction was complete (LC-MS), the mixture was concentrated to afford the final compound (51 mg). Compound 2 was characterized by ¹H NMR, LC-MS and HPLC. ¹H NMR (D₂O), δ: 3.62-3.92 (m, 10H), 1.99-2.05 (m, 1H), 1.54-1.60 (m, 1H), 1.41 (d, J=7.12 Hz, 2H), 1.26-1.33 (m, 1H), 1.06 (d, J=6.92 Hz, 3H), 0.99 (t, J=7.38 Hz, 3H). LC-MS, (M+1), 272. HPLC (>95%, retention time, 1.94 min).

### Example 3

### Preparation of Compound 3

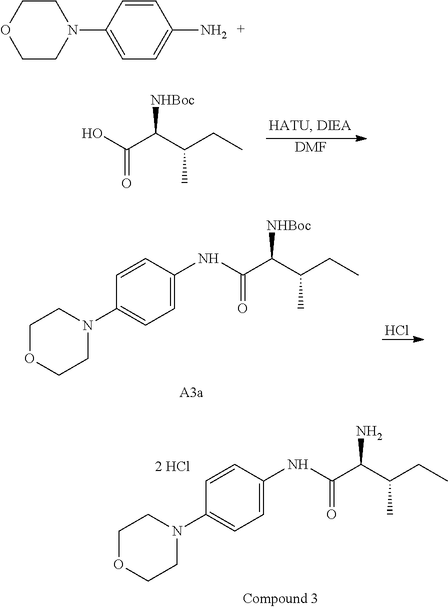

### Preparation of Intermediate A3a

| S. | | | | | |
|---|---|---|---|---|---|
| No. | Chemicals/Reagents & Solvents | MW | mmol | Eq. | Amts |
| 1 | Boc-Ile-OH | 231.29 | 0.432 | 1.0 | 100 mg |
| 2 | DMF (anhydrous) | | | | 3 mL |
| 3 | DIEA, d = 0.742 | 129.25 | 1.08 | 2.5 | 0.20 mL |
| 4 | HATU | 380.2 | 0.43 | 1.0 | 163 mg |
| 5 | 4-morpholinoaniline | 178.24 | 0.432 | 1.0 | 77 mg |

To a solution of Boc-Ile-OH in DMF were added HATU, 4-morpholinoaniline, and DIEA in that order. The reaction mixture was stirred for 1 hour at room temperature.

After the reaction was complete (LC-MS), saturated aqueous NaHCO₃ was added to quench the reaction and EtOAc was used to extract the product. The organic layer was separated and washed with water, brine, dried with anhydrous Na₂SO₄, filtered, and concentrated. The intermediate A3a was obtained as an off-white solid (166 mg).

### Preparation of Compound 3

| S. No. | Chemicals/Reagents & Solvents | MW | mmol | Eq. | Amts |
|---|---|---|---|---|---|
| 1 | A3a | | | | 160 mg |
| 2 | HCl in MeOH | | | | 4 mL |

Cold HCl solution in MeOH was added to the residue of A3a. The reaction mixture was stirred for 3 hours at room temperature.

After the reaction was complete (LC-MS), the mixture was concentrated to afford the final compound 3 (120 mg). Compound 3 was characterized by ¹H NMR, LC-MS and HPLC. ¹H NMR (MeOD), δ: 7.88 (d, J=9.16 Hz, 2H), 7.69 (d, J=9.12 Hz, 2H), 4.09-4.11 (m, 4H), 3.92 (d, J=5.80 Hz, 1H), 3.66-3.69 (m, 4H), 2.03-2.10 (m, 1H), 1.62-1.69 (m, 1H), 1.24-1.32 (m, 1H), 1.11 (d, J=6.92 Hz, 3H), 1.01 (t, J=7.40 Hz, 3H). LC-MS, (M+1), 292. HPLC (>95%, retention time, 4.86 min)

### Example 4

### Preparation of Compound 4

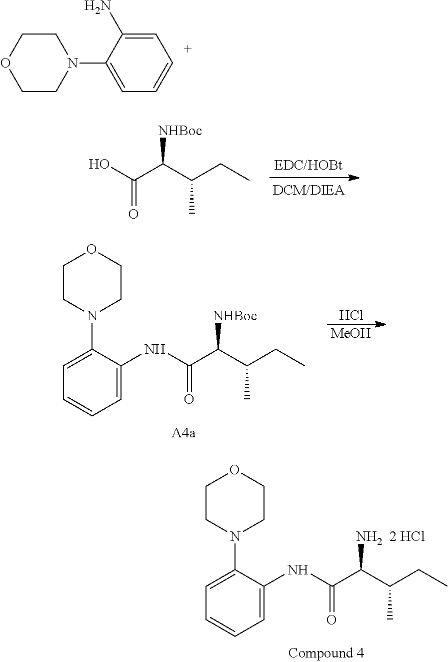

### Preparation of Intermediate A4a

| | Chemicals/ | | | | |
|---|---|---|---|---|---|
| S. No. | Reagents & Solvents | MW | mmol | Eq. | Amts |
| 1 | Boc-Ile-OH | 231.29 | 1 | 1.0 | 231 mg |
| 2 | DCM (anhydrous) | | | | 5 mL |
| 3 | DIEA, d = 0.742 | 129.25 | 2.5 | 2.5 | 0.44 mL |
| 4 | HOBt•H₂O | 135.13 | 1 | 1.0 | 135 mg |
| 5 | EDC | 191.7 | 1 | 1.0 | 192 mg |
| 6 | 2-morpholinoaniline | 178.23 | 0.96 | 0.96 | 170 mg |

To the solution of Boc-Ile-OH in DCM were added HOBt, EDC, DIEA, and 2-morpholinoaniline in that order. The reaction mixture was stirred for 7.5 hours at room temperature.

The reaction mixture was subject to prep-HPLC purification to afford the intermediate A4a (94 mg).

### Preparation of Compound 4

| S. No. | Chemicals/Reagents & Solvents | MW | mmol | Eq. | Amts |
|---|---|---|---|---|---|
| 1 | A4a | | | | 60 mg |
| 2 | HCl in MeOH | | | | 4 mL |

Cold HCl solution in MeOH was added to the residue of A4a. The reaction mixture was stirred for 3 hours at room temperature.

After the reaction was complete (LC-MS), the mixture was concentrated to afford the final compound 4 (73 mg). Compound 4 was characterized by ¹H NMR, LC-MS and HPLC. ¹H NMR (D2O), δ: 7.67 (dd, J=7.96 and 1.48 Hz, 1H), 7.49 (dd, J=8.10 and 1.34 Hz, 1H), 7.42 (dt, J=7.75 and 1.53 Hz, 1H), 7.34 (dt, J=7.67 and 1.43 Hz, 1H), 4.22 (d, J=5.00 Hz, 1H), 3.95-3.97 (m, 4H), 3.13-3.15 (m, 4H), 2.11-2.17 (m, 1H), 1.57-1.64 (m, 1H), 1.30-1.39 (m, 1H), 1.12 (d, J=6.96 Hz, 3H), 1.00 (t, J=7.40 Hz, 3H). LC-MS, (M+1), 292. HPLC (>95%, retention time, 5.31 min).

### Example 5

### Preparation of Compound 5

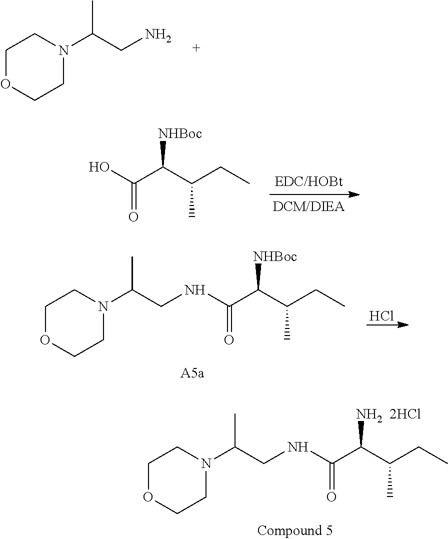

### Preparation of Intermediate A5a

| S. | | | | | |
|---|---|---|---|---|---|
| No. | Chemicals/Reagents & Solvents | MW | mmol | Eq. | Amts |
| 1 | Boc-Ile-OH | 231.29 | 1 | 1.0 | 231 mg |
| 2 | DCM (anhydrous) | | | | 5 mL |
| 3 | DIEA, d = 0.742 | 129.25 | 2.5 | 2.5 | 0.44 mL |
| 4 | HOBt•H₂O | 135.13 | 1 | 1.0 | 135 mg |
| 5 | EDC | 191.7 | 1 | 1.0 | 192 mg |
| 6 | 2-morpholin-4-yl-propylamine | 144.22 | | | 0.19 mL |

To the solution of Boc-Ile-OH in DCM were added HOBt, EDC, DIEA, and 2-morpholin-4-yl-propylamine in that order. The reaction mixture was stirred for 3 hours at room temperature.

The reaction mixture was subject to prep-HPLC purification to afford the intermediate A5a (219 mg).

### Preparation of Compound 5

| S. No. | Chemicals/Reagents & Solvents | MW | mmol | Eq. | Amts |
|---|---|---|---|---|---|
| 1 | A5a | | | | 210 mg |
| 2 | HCl in MeOH | | | | 4 mL |

Cold HCl solution in MeOH was added to the residue of A4a. The reaction mixture was stirred for 3 hours at room temperature.

After the reaction was complete (LC-MS), the mixture was purified by a prep-HPLC and converted to an HCl salt by a treatment with HCl in MeOH to afford final compound 5 (120 mg). Compound 5 was characterized by ¹H NMR, LC-MS and HPLC. ¹H NMR (D2O), δ: 3.30-4.24 (m, 12H), 1.92-2.05 (m, 1H), 1.43-1.56 (m, 1H), 1.36-1.42 (m, 3H), 1.16-1.30 (m, 1H), 1.01 (d, J=6.92 Hz, 3H), 0.94 (d, J=7.34 Hz, 3H). LC-MS, (M+1), 258. HPLC (>95%, retention time, 1.41 min).

### Example 6

### Preparation of Compound 6

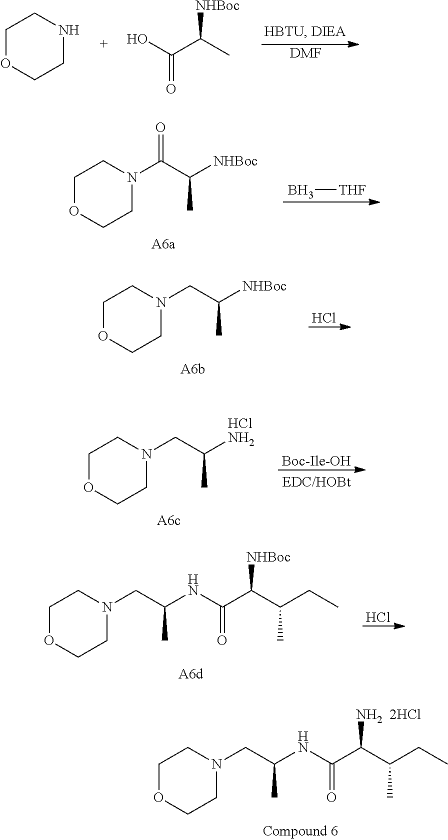

### Preparation of Intermediate A6a

| S. | | | | | |
|---|---|---|---|---|---|
| No. | Chemicals/Reagents & Solvents | MW | mmol | Eq. | Amts |
| 1 | Boc-Ala-OH | 189.21 | 2 | 1.0 | 378 mg |
| 2 | DMF (anhydrous) | | | | 5 mL |
| 3 | DIEA, d = 0.742 | 129.25 | 5 | 2.5 | 0.87 mL |
| 4 | HBTU | 379 | 2 | 1.0 | 758 mg |
| 5 | Morpholine, d = 0.99 | 87.12 | 2 | 1.0 | 175 uL |

To a solution of Boc-Ala-OH in DMF were added HBTU, DIEA, and morpholine in that order. The reaction mixture was stirred for 2 hours at room temperature.

After the reaction was complete (LC-MS), the reaction mixture was subject to prep-HPLC purification to afford the intermediate A6a (333 mg).

### Preparation of Intermediate A6b

| | Chemicals/Reagents & | | | | | |
|---|---|---|---|---|---|---|
| S. No. | Solvents | MW | mmol | Eq. | Amts | |
| 1 | A6a | 258.31 | 1.67 | 1.0 | 430 | mg |
| 2 | THF, anhydrous | | | | 10 | mL |
| 3 | BH₃—THF, 1M | | | 2.0 | 3.3 | mL |

A6a was dissolved in THF. BH₃-THF was added slowly to the above solution. Bubbles were observed and the reaction mixture was stirred over night at RT.

After the reaction was complete (LC-MS), the mixture was quenched carefully with water and concentrated. The mixture was further purified by prep-HPLC to afford intermediate A6b (192 mg).

### Preparation of Intermediate A6c

| | | | | | |
|---|---|---|---|---|---|
| S. No. | Chemicals/Reagents & Solvents | MW | mmol | Eq. | Amts |

| | | | |
|---|---|---|---|
| 1 | A6b | 210 | mg |
| 2 | HCl in MeOH | 4 | mL |

Cold HCl solution in MeOH was added to the residue of A6b. The reaction mixture was stirred for 3 hours at room temperature.

After the reaction was complete (LC-MS), the mixture was concentrated to afford the intermediate A6c (158 mg).

### Preparation of Intermediate A6d

| | Chemicals/Reagents & | | | | | |
|---|---|---|---|---|---|---|
| S. No. | Solvents | MW | mmol | Eq. | Amts | |
| 1 | Boc-Ile-OH | 231.29 | 0.8 | 1.0 | 180 | mg |
| 2 | DMF (anhydrous) | | | | 3 | mL |
| 3 | DIEA, (d = 0.742 | 129.25 | 1.6 | 2.0 | 0.28 | mL |
| 4 | HOBt•H₂O | 135.13 | 0.8 | 1.0 | 108 | mg |
| 5 | EDC | 191.7 | 0.8 | 1.0 | 153 | mg |
| 6 | A6c | 180 | 0.83 | | 150 | mg |

To the solution of Boc-Ile-OH and A6c in DCM were added HOBt, EDC, and DIEA in that order. The reaction mixture was stirred for 1 hour at room temperature.

The reaction mixture was subject to prep-HPLC purification to afford the intermediate A6d (86 mg).

### Preparation of Compound 6

| | | | | | |
|---|---|---|---|---|---|
| S. No. | Chemicals/Reagents & Solvents | MW | mmol | Eq. | Amts |

| | | | |
|---|---|---|---|
| 1 | A6d | 60 | mg |
| 2 | 4M HCl in H₂O | 4 | mL |

Cold TFA/DCM was added to the residue of A6d. The reaction mixture was stirred for 3 hours at room temperature.

After the reaction was complete (LC-MS), the mixture was concentrated to afford the final compound 6 (89 mg). Compound 6 was characterized by ¹H NMR, LC-MS and HPLC. ¹H NMR (D2O), δ: 4.21-4.32 (m, 1H), 3.88-4.11 (m, 2H), 3.65-3.87 (m, 3H), 3.34-3.53 (m, 2H), 3.07-3.34 (m, 4H), 1.83-1.95 (m, 1H), 1.30-1.41 (m, 1H), 1.21 (d, J=6.80 Hz, 3H), 1.04-1.17 (m, 1H), 0.91 (d, J=6.96 Hz, 3H), 0.82 (t, J=7.38 Hz, 3H). LC-MS, (M+1), 258. HPLC (>95%, retention time, 1.59 min).

### Example 7

### Preparation of Compound 7

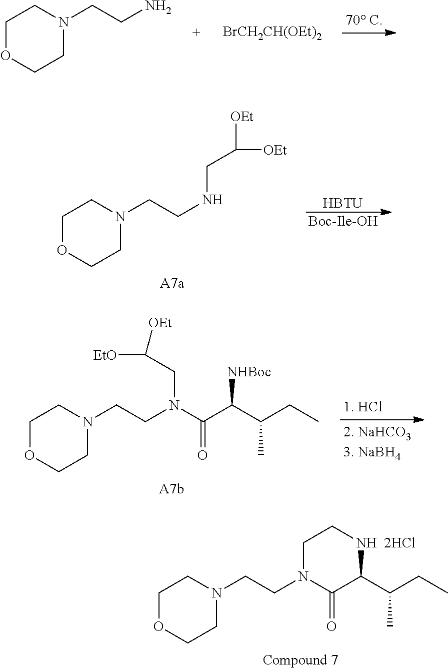

### Preparation of Intermediate A7a

| S. No. | Chemicals/Reagents & Solvents | MW | mmol | Eq. | Amts |
|---|---|---|---|---|---|
| 1 | 2-Morpholin-4-yl-ethylamine | 130.11 | 30 | 3 | 4 mL |
| 2 | BrCH₂CH(OEt)₂ | 197.07 | 10 | 1 | 1.5 mL |

Amine and bromide were mixed and heated up to 70° C. for 5 hours with stirring.

LC-MS showed that the product contains a mixture of non-mono-, and bis-alkylated amines. The mixture was used in next step without further purification (5.5 ml).

### Preparation of Intermediate A7b

| | Chemicals/Reagents & | | | | | |
|---|---|---|---|---|---|---|
| S. No. | Solvents | MW | mmol | Eq. | Amts | |
| 1 | Boc-Ile-OH | 231.29 | 30 | 1.0 | 7 | g |
| 2 | A7a | | | | 5.5 | mL |
| 2 | Acetonitrile (anhydrous) | | | | 100 | mL |
| 3 | DIEA, d = 0.742 | 129.25 | 75 | 2.5 | 13 | mL |
| 4 | HBTU | 379.3 | 30 | 1.0 | 11.4 | g |

HBTU was added to a solution of Boc-Ile-OH in acetonitrile and then DIEA was added. After the mixture was stirred at room temperature for 10 minutes, A7a was then added. The reaction mixture was stirred for another 2 hours.

After the reaction was complete (LC-MS), portions of solution were subjected to prep-HPLC separation, collecting fraction peak with M⁺=460 (A7b, 2 g).

### Preparation of Compound 7

A7b was dissolved in cold HCl in MeOH. The reaction mixture was stirred 3 hrs at RT. After the reaction was complete (LC-MS), the mixture was concentrated to afford an HCl salt intermediate (209 mg).

The above HCl salt intermediate was dissolved in a mixed solvent of acetonitrile and water, neutralized with saturated sodium bicarbonate till pH˜7. The reaction mixture was stirred for 2.5 hrs at RT. Then, excess NaBH₄ was added and the mixture was stirred overnight. However, not much product was observed by LC-MS the second day morning, thus, the reaction mixture was heated to 45° C. for 7.5 hrs.

After the reaction was complete (LC-MS), the mixture was filtered and subject to prep-HPLC purification to afford the desired final compound. The final compound was converted to an HCl salt by treatment with HCl in MeOH (43 mg). Compound 7 was characterized by ¹H NMR, LC-MS and HPLC. ¹H NMR (D₂O), δ: 2.90-4.20 (m, 17H), 1.84-2.35 (m, 1H), 1.07-1.53 (m, 2H), 0.80-1.02 (m, 6H). LC-MS, (M+1), 270. HPLC (>95%, retention time, 1.27 min).

### Example 8

### Preparation of Compound 8

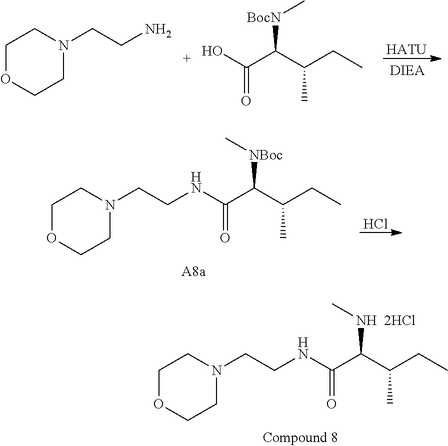

### Preparation of Intermediate A8a

| | Chemicals/Reagents & | | | | | |
|---|---|---|---|---|---|---|
| S. No. | Solvents | MW | mmol | Eq. | Amts | |
| 1 | N-methyl-Boc-Ile-OH | 245.32 | 2 | 2 | 490 | mg |
| 2 | 2-morpholin-4-yl-ethylamine | 130.19 | 1 | 1 | 0.13 | mL |
| 3 | Acetinitrile (anhydrous) | | | | 10 | mL |
| 4 | DIEA, d = 0.742 | 129.25 | 3 | 3 | 0.52 | mL |
| 5 | HATU | 380.2 | 2 | 2 | 760 | mg |

To a solution of acid and HATU solution in acetonitrile, DIEA was added with stirring. After 20 minutes, amine was added and the reaction was continued to stir for 1 hour.

LC-MS showed the completion of the reaction. The reaction solution was subjected to prep-HPLC separation (A8a, 65 mg).

### Preparation of Compound 8

| | Chemicals/Reagents & | | | | | |
|---|---|---|---|---|---|---|
| S. No. | Solvents | MW | mmol | Eq. | Amts | |
| 1 | A8a | 357.49 | 0.18 | 1 | 65 | mg |
| 2 | Methanol | | | | 10 | mL |
| 3 | 12N HCl | | | | 5 | mL |

To a solution of A8a in methanol, HCl aqueous solution was added and stirred at room temperature for 2 hours.

LC-MS showed the completion of the reaction. The solvents were removed in vacuum to afford compound 8 (60 mg). Compound 8 was characterized by ¹H NMR, LC-MS and HPLC. ¹H NMR (MeOD), δ: 3.82-4.11 (m, 5H), 3.78 (d, J=4.52 Hz, 1H), 3.68 (d, J=11.2 Hz, 1H), 3.45-3.59 (m, 2H), 3.11-3.41 (m, 5H), 2.78 (s, 3H), 1.93-2.05 (m, 1H), 1.56-1.68 (m, 1H), 1.15-1.32 (m, 1H)), 0.96-1.09 (m, 6H). LC-MS, (M+1), 258. HPLC (>95%, retention time, 1.0 min).

### Example 9

### Preparation of Compound 9

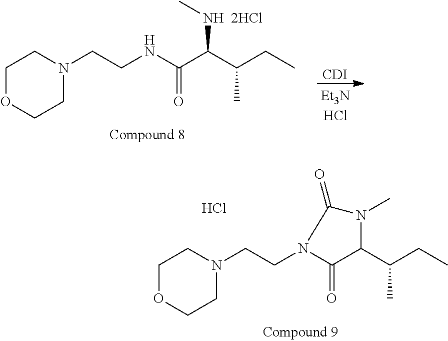

| | Chemicals/Reagents & | | | | | |
|---|---|---|---|---|---|---|
| S. No. | Solvents | MW | mmol | Eq. | Amts | |
| 1 | Compound 8 | 330.29 | 2.27 | 1 | 750 | mg |
| 2 | CDI | 160.17 | 20 | 8.8 | 3.2 | g |
| 3 | Acetinitrile (anhydrous) | | | | 15 | mL |
| 4 | Et₃N | 101.19 | 19.7 | 8.7 | 2 | g |

To a solution of Compound 8 and CDI in acetonitrile, Et₃N was added with stirring. The stirring was continued for three (3) days.

LC-MS showed the completion of the reaction. Methanol was added to quench the reaction. After all the volatile solvent was removed, the residue was redissolved in methanol and subjected to prep-HPLC separation. After removal of the solvent, the product formic acid salt was converted to HCl salt by co-evaporating with 25 ml 1.25 N HCl methanol solution three times (76 mg). Compound 9 was characterized by ¹H NMR, LC-MS and HPLC. ¹H NMR (D₂O), δ: 3.68-4.14 (m, 7H), 3.00-3.63 (m, 6H), 2.68 (d, J=8.40 Hz, 3H), 1.91-2.03 (m, 1H), 0.98-1.51 (m, 2H), 0.90 (d, J=7.00 Hz, 1H), 0.79-0.88 (m, 3H), 0.71 (d, J=6.92 Hz, 1H). LC-MS, (M+1), 284. HPLC (>95%, retention time, 4.32 min).

### Example 10

### Preparation of Compound 10

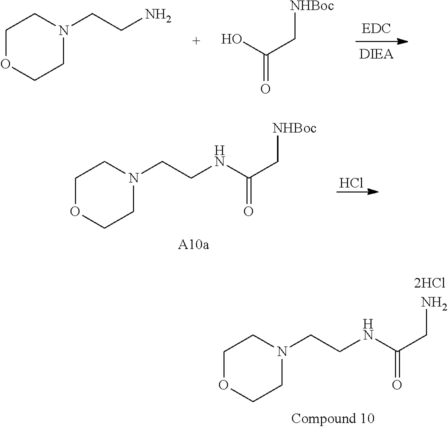

### Preparation of Intermediate A10a

| | Chemicals/Reagents & | | | | | |
|---|---|---|---|---|---|---|
| S. No. | Solvents | MW | mmol | Eq. | Amts | |
| 1 | Boc-Gly-OH | 175.18 | 3 | 1 | 526 | mg |
| 2 | 2-morpholin-4-yl-ethylamine | 130.19 | 3 | 1 | 0.39 | mL |
| 3 | Acetinitrile (anhydrous) | | | | 10 | mL |
| 4 | DIEA, d = 0.742 | 129.25 | 6 | 2 | 1.2 | mL |
| 5 | EDC | 191.7 | 4.17 | 1.4 | 800 | mg |

A mixture of Boc-Gly-OH and EDC was suspended in acetonitrile. DIEA was added, followed by the addition of 2-Morpholino-4-yl-ethylamine. The reaction mixture was stirred for 2 hours.

LC-MS showed the completion of the reaction. The reaction mixture was subjected to prep-HPLC separation to give pure A10a (177 mg).

### Preparation of Compound 10

| | Chemicals/Reagents & | | | | | |
|---|---|---|---|---|---|---|
| S. No. | Solvents | MW | mmol | Eq. | Amts | |
| 1 | A10a | 357.49 | 0.18 | 1 | 177 | mg |
| 2 | Methanol | | | | 50 | mL |
| 3 | 12N HCl | | | | 25 | mL |

To a solution of A10a in methanol, HCl aqueous solution was added and stirred at room temperature for 2 hours.

LC-MS showed the completion of the reaction. The solvents were removed in vacuum to afford Compound 10 (160 mg). Compound 10 was characterized by ¹H NMR, LC-MS and HPLC. ¹H NMR (MeOD), δ: 3.93-4.08 (m, 4H), 3.77 (s, 2H), 3.56-3.72 (m, 4H), 3.32-3.36 (m, 3H), 3.09-3.24 (m, 2H). LC-MS, (M+1), 188. HPLC (>95%, retention time, 1.0 min).

### Example 11

### Preparation of Compound 11

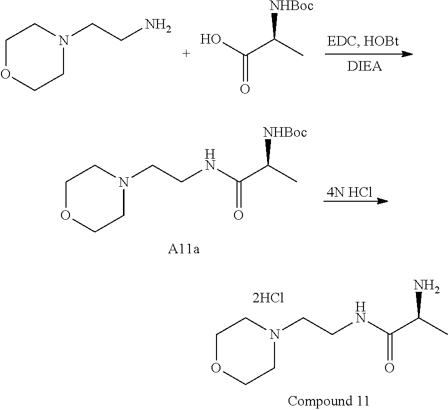

### Preparation of Intermediate A11a

| | Chemicals/Reagents & | | | | | |
|---|---|---|---|---|---|---|
| S. No. | Solvents | MW | mmol | Eq. | Amts | |
| 1 | Boc-Ala-OH | 189.21 | 1 | 1 | 190 | mg |
| 2 | 2-morpholin-4-yl-ethylamine | 130.19 | 1 | 1 | 0.13 | mL |
| 3 | Acetinitrile (anhydrous) | | | | 5 | mL |
| 4 | BtoH•H₂O | 153.14 | 1 | 1 | 153 | mg |
| 5 | DIEA, d = 0.742 | 129.25 | 2 | 2 | 0.38 | mL |
| 6 | EDC | 191.7 | 1 | 1 | 192 | mg |

A mixture of Boc-Ala-OH, BtOH.H₂O, and DIEA was suspended in acetonitrile. EDC was then added with stirring and the solution became clear. 2-Morpholin-4-yl-ethylamine was added and stirred for 2 hours at room temperature.

LC-MS showed the completion of the reaction. The reaction mixture was subjected to prep-HPLC separation to give pure A11a (66 mg).

### Preparation of Compound 11

| | Chemicals/Reagents & | | | | | |
|---|---|---|---|---|---|---|
| S. No. | Solvents | MW | mmol | Eq. | Amts | |
| 1 | A11a | 301.38 | 0.22 | 1 | 66 | mg |
| 2 | Methanol | | | | 50 | mL |
| 3 | 12N HCl | | | | 25 | mL |

To a solution of A11a in methanol, HCl aqueous solution was added and stirred at room temperature for 2 hours.

LC-MS showed the completion of the reaction. The solvents were removed in vacuo to afford Compound 11 (60 mg). Compound 11 was characterized by ¹H NMR, LC-MS and HPLC. ¹H NMR (D₂O), δ: 4.10-4.27 (m, 3H), 3.87-4.01 (m, 2H), 3.77-3.88 (m, 1H), 3.60-3.76 (m, 3H) 3.40-3.55 (m, 2H), 3.26-3.39 (m, 2H), 1.60 (d, J=7.16 Hz, 3H). LC-MS, (M+1), 202. HPLC (>95%, retention time, 1.0 min).

### Example 12

### Preparation of Compound 12

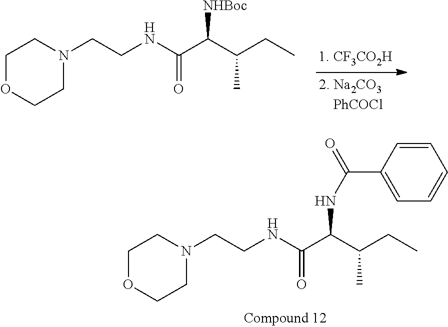 [SMILES: CC[C@H](C)[C@H](NC(=O)OC(C)(C)C)C(=O)NCCN1CCOCC1 ; CC[C@H](C)[C@H](NC(=O)c1ccccc1)C(=O)NCCN1CCOCC1]

| | Chemicals/Reagents & | | | | | |
|---|---|---|---|---|---|---|
| S. No. | Solvents | MW | mmol | Eq. | Amts | |
| 1 | 2-Boc-amino-3-methyl-N-(2- | 343.46 | 1 | 1 | 343 | mg |
| | morpholinoethyl)pentamide | | | | | |
| 2 | CF₃CO₂H | | | | 10 | mL |
| 3 | CH₂Cl₂ | | | | 10 | mL |
| 4 | THF | | | | 10 | mL |
| 5 | Water | | | | 10 | mL |
| 6 | Na₂CO₃ | 105.99 | 10 | 10 | 1.06 | g |
| 7 | PhCOCl | 140.57 | 2 | 2 | 280 | mg |

2-Boc-amino-3-methyl-N-(2-morpholinoethyl)pentamide was dissolved in CF₃CO₂H/CH₂Cl₂ and stirred at room temperature for 2 hours. After LC-MS showed the complete deprotection of the starting material, the solvents were removed under vacuum. The residue was dissolved in THF/H₂O solution and Na₂CO₃ was added to adjust the solution to basic. Benzoyl chloride was then added with stirring for 2 hours.

LC-MS showed the completion of the reaction. THF was then removed under vacuum and the aqueous solution was extracted with AcOEt. AcOEt was dried over Na₂SO₄ and removed. The residue was dissolved in methanol and subjected to prep-HPLC separation. After removal of the solvent, the product formic acid salt was converted to HCl salt by co-evaporating with 50 mL 1.25 HCl methanol solution three times (91 mg). Compound 12 was characterized by ¹H NMR, LC-MS and HPLC. ¹H NMR (MeOD), δ: 7.86-7.91 (m, 2H), 7.56-7.61 (m, 1H), 7.47-7.53 (m, 2H), 4.20 (d, J=7.96 Hz, 1H), 4.00-4.10 (m, 2H), 182-3.92 (m, 2H), 3.71-3.80 (m, 1H), 3.59-3.69 (m, 2H), 3.15-3.56 (m, 5H), 1.96-2.08 (m, 1H), 1.63-1.75 (m, 1H), 1.26-1.39 (m, 1H), 0.94-1.06 (m, 6H). LC-MS, (M+1), 348. HPLC (>95%, retention time, 5.27 min).

### Example 13

### Preparation of Compound 13

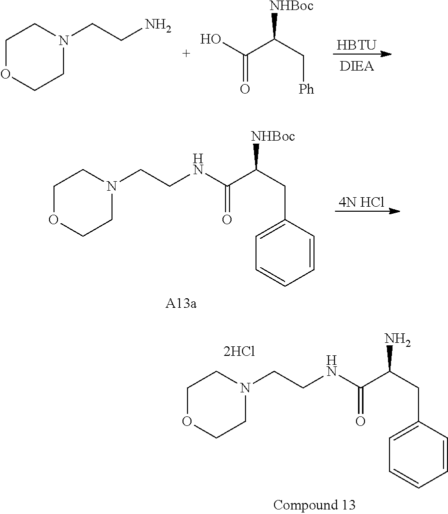

### Preparation of Intermediate A13a

| | Chemicals/Reagents & | | | | | |
|---|---|---|---|---|---|---|
| S. No. | Solvents | MW | mmol | Eq. | Amts | |
| 1 | Boc-Phe-OH | 256.31 | 1 | 1 | 256 | mg |
| 2 | 2-morpholin-4-yl-ethylamine | 130.19 | 1 | 1 | 0.13 | mL |
| 3 | Acetonitrile (anhydrous) | | | | 5 | mL |
| 4 | DIEA, d = 0.742 | 129.25 | 1 | 1 | 0.17 | mL |
| 5 | HBTU | 379.25 | 1 | 1 | 379 | mg |

To a solution of Boc-Phe-OH, HBTU in acetonitrile was added DIEA. After stirring for 5 minutes, 2-morpholin-4-yl-ethylamine was added and the reaction mixture was then stirred for another 2 hours.

LC-MS showed the completion of the reaction. The reaction mixture was subjected to prep-HPLC separation to give pure A13a (210 mg).

### Preparation of Compound 13

| S. No. | Chemicals/Reagents & Solvents | MW | mmol | Eq. | Amts |
|---|---|---|---|---|---|
| 1 | A13a | 377.48 | 0.56 | 1 | 210 mg |
| 2 | Methanol | | | | 50 mL |
| 3 | 12N HCl | | | | 25 mL |

To a solution of A13a in methanol, HCl aqueous solution was added and stirred at room temperature for 2 hours.

LC-MS showed the completion of the reaction. The solvents were removed in vacuum to afford Compound 13 (195 mg). Compound 13 was characterized by ¹H NMR, LC-MS and HPLC. ¹H NMR (D₂O), δ: 7.32-7.41 (m, 2H), 7.43-7.57 (m, 3H), 4.30 (t, J=7.32 Hz, 1H), 3.94-4.10 (m, 4H), 3.61 (t, J=6.34 Hz, 2H), 3.08-3.44 (m, 9H). LC-MS, (M+1), 278. HPLC (>95%, retention time, 1.21 min).

A number of compounds, including Compound 14, Compound 15, Compound 16, Compound 17, and Compound 18, were prepared according to the method of preparation of Compound 13, substituting the appropriate starting materials in place of Boc-Phe-OH.

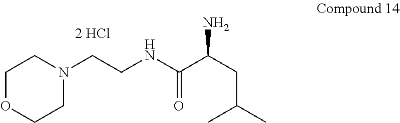 [SMILES: CC(C)C[C@H](N)C(=O)NCCN1CCOCC1]

Compound 14 was prepared according to the method of preparation of Compound 13, except that Boc-Phe-OH was replaced with Boc-Leu-OH. Compound 14 (175 mg) was characterized by ¹H NMR, LC-MS and HPLC. ¹H NMR (D₂O), δ: 4.07-410 (m, 2H), 4.03 (d, J=7.22 Hz, 1H), 3.71-3.93 (m, 3H), 3.50-3.70 (m, 3H), 3.18-3.44 (m, 4H), 1.74 (t, J=7.26 Hz, 2H), 1.61-1.72 (m, 1H), 0.89-1.03 (m, 6H). LC-MS, (M+1), 244. HPLC (>95%, retention time, 1.0 min).

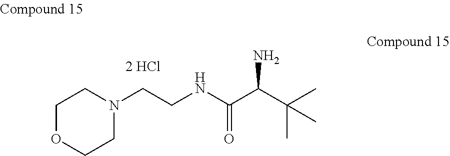 [SMILES: CC(C)(C)[C@H](N)C(=O)NCCN1CCOCC1]

Compound 15 was prepared according to the method of preparation of Compound 13, except that Boc-Phe-OH was replaced with Boc-D-t-butylglycine-OH. Compound 15 (120 mg) was characterized by ¹H NMR, LC-MS and HPLC. ¹H NMR (D₂O), δ: 3.91-4.10 (m, 2H), 3.62-3.82 (m, 3H), 3.60 (s, 1H), 3.38-3.58 (m, 3H), 3.05-3.34 (m, 4H), 0.96 (s, 9H). LC-MS, (M+1), 244. HPLC (>95%, retention time, 1.0 min).

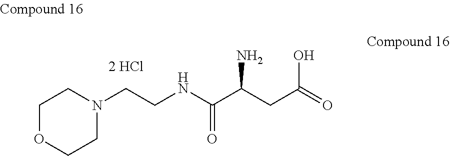 [SMILES: N[C@@H](CC(=O)O)C(=O)NCCN1CCOCC1]

Compound 16 was prepared according to the method of preparation of Compound 13, except that Boc-Phe-OH was replaced with Boc-Asp(OTBU)-OH. Compound 16 (50 mg) was characterized by ¹H NMR, LC-MS and HPLC. ¹H NMR (D₂O), δ: 4.23 (t, J=6.24 Hz, 1H), 3.91-4.11 (m, 2H), 3.67-3.84 (m, 2H), 3.55-3.67 (m, 2H), 3.41-3.54 (m, 2H), 3.27 (t, J=6.32 Hz, 2H), 3.06-3.22 (m, 2H), 2.85-3.01 (m, 2H). LC-MS, (M+1), 246. HPLC (>95%, retention time, 1.0 min).

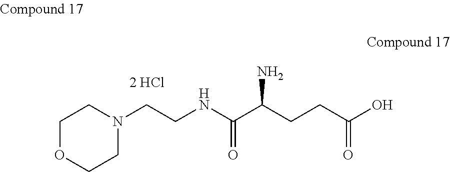 [SMILES: N[C@@H](CCC(=O)O)C(=O)NCCN1CCOCC1]

Compound 17 was prepared according to the method of preparation of Compound 13, except that Boc-Phe-OH was replaced with Boc-Glu(OTBU)-OH. Compound 17 (60 mg) was characterized by ¹H NMR, LC-MS and HPLC. ¹H NMR (D₂O), δ: 3.94-4.10 (m, 2H), 3.97 (t, J=6.56 Hz, 1H), 3.67-3.83 (m, 2H), 3.60-3.69 (m, 1H), 3.40-3.59 (m, 3H), 3.20 (t, J=6.78 Hz, 2H), 3.08-3.23 (m, 2H), 2.45 (t, J=7.18 Hz, 2H), 2.01-2.17 (m, 2H). LC-MS, (M+1), 260. HPLC (>95%, retention time, 1.0 min).

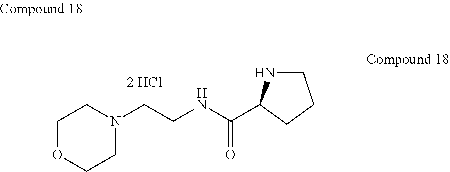 [SMILES: O=C(NCCN1CCOCC1)[C@@H]1CCCN1]

Compound 17 was prepared according to the method of preparation of Compound 13, except that Boc-Phe-OH was replaced with Boc-Pro-OH. Compound 18 (80 mg) was characterized by ¹H NMR, LC-MS and HPLC. ¹H NMR (D₂O), δ: 4.27 (t, J=7.38 Hz, 1H), 3.91-4.10 (m, 2H), 3.60-3.81 (m, 3H), 3.37-3.57 (m, 3H), 3.19-3.37 (m, 4H), 3.04-3.19 (m, 2H), 2.25-2.40 (m, 1H), 1.85-2.02 (m, 3H). LC-MS, (M+1), 228. HPLC (>95%, retention time, 1.0 min).

### Example 19

### Preparation of Compound 19

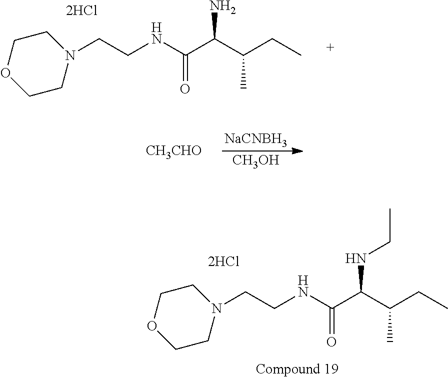 [SMILES: CC[C@H](C)[C@H](N)C(=O)NCCN1CCOCC1 ; "NaCNBH3" ; "CH3CHO" ; "CH3OH" ; CCN[C@H](C(=O)NCCN1CCOCC1)[C@@H](C)CC]

| S. | | | | | |
|---|---|---|---|---|---|
| No. | Chemicals/Reagents & Solvents | MW | mmol | Eq. | Amts |
| 1 | (2S,3S)-2-amino-3-methyl-N-(2- | 316.27 | 1 | 1 | 314 mg |
| | morpholinoethyl)pentanamide | | | | |
| 2 | CH₃CHO | 44.05 | 1 | 1 | 44 mg |
| 3 | NaCNBH₃ (1M in THF) | | 1 | 1 | 1 mL |
| 4 | Methanol | 105.99 | 10 | 10 | 1.06 g |

A mixture of (2S,3S)-2-amino-3-methyl-N-(2-morpholinoethyl)pentanamide and acetaldehyde was dissolved in methanol and NaCNBH₃ was added. The reaction mixture was then stirred overnight.

LC-MS showed the completion of the reaction. K₂CO₃ (270 mg) was added to quench the reaction. The precipitate was dissolved by adding 2 mL water and then the solution was subjected to prep-HPLC separation. Then the formic acid salt was transformed to HCl salt by co-evaporating with HCl/methanol (1N) solution. Compound 19 (50 mg) was characterized by ¹H NMR and LC-MS. ¹H NMR (D₂O), δ: 4.05-4.23 (m, 2H), 3.48-3.94 (m, 7H), 3.38 (t, J=6.82 Hz, 2H), 3.17-3.34 (m, 2H), 3.08 (q, J=7.32 Hz, 2H), 1.94-2.07 (m, 1H), 1.44-1.60 (m, 1H), 1.30 (t, J=7.32 Hz, 1H), 1.14-1.26 (m, 3H), 0.89-1.06 (m, 6H). LC-MS, (M+1), 272.

### Example 20

### Preparation of Compound 20

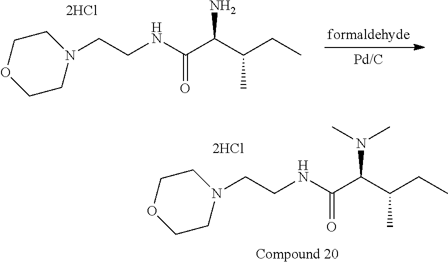 [SMILES: CC[C@H](C)[C@H](N)C(=O)NCCN1CCOCC1 ; CC[C@H](C)[C@@H](C(=O)NCCN1CCOCC1)N(C)C]

| S. | | | | | |
|---|---|---|---|---|---|
| No. | Chemicals/Reagents & Solvents | MW | mmol | Eq. | Amts |
| 1 | (2S,3S)-2-amino-3-methyl-N-(2- | 316.27 | 1 | 1 | 316 mg |
| | morpholinoethyl)pentanamide | | | | |
| 2 | Formaldehyde (37% solution) | 44.05 | 16.8 | 16.8 | 2 mL |
| 3 | Pd/C (10%) | | | | 1 g |
| 4 | H₂O | | | | 10 mL |

A mixture of (2S,3S)-2-amino-3-methyl-N-(2-morpholinoethyl)pentanamide, formaldehyde, and Pd/C in water was hydrogenated at 50 Psi for 5 hours.

LC-MS showed the completion of the reaction. Pd/C was filtrated off and the clear solution was subjected to prep-HPLC separation. The formic acid salt form was transformed to HCl salt (220 mg) by co-evaporating with HCl/methanol (1N) solution. Compound 20 (220 mg) was characterized by ¹H NMR, LC-MS and HPLC. ¹H NMR (D₂O), δ: 3.70-4.13 (m, 4H), 3.64-3.69 (m, 1H), 3.56-3.64 (m, 2H), 3.05-3.52 (m, 6H), 2.80 (s, 6H), 2.02-2.15 (m, 1H), 1.32-1.46 (m, 1H), 1.01-1.15 (m, 1H), 0.81-0.93 (m, 6H). LC-MS, (M+1), 272. HPLC (>95%, retention time, 1.14 min).

### Example 21

### Preparation of Compound 21

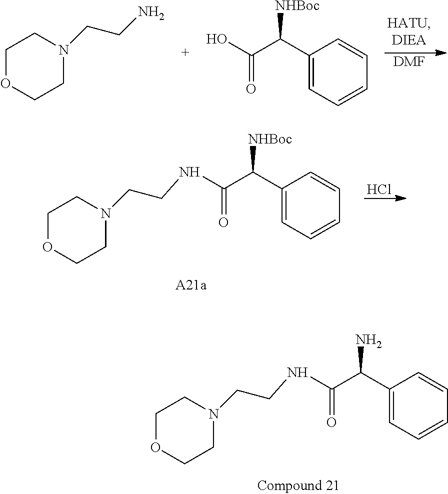

### Preparation of Intermediate A11a

| | Chemicals/Reagents & | | | | |
|---|---|---|---|---|---|
| S. No. | Solvents | MW | mmol | Eq. | Amts |
| 1 | Boc-L-alpha-phenylglycine | 251.28 | 1 | 1.0 | 251 mg |
| 2 | DMF (anhydrous) | | | | 6 mL |
| 3 | DIEA, d = 0.742 | 129.25 | 2.0 | 2.0 | 0.35 mL |
| 4 | HATU | 380.2 | 1 | 1.0 | 380 mg |
| 5 | Amine, d = 1 | 130.19 | 2 | 2.0 | 0.13 mL |

To a solution of acid in DMF were added HATU, DIEA, and amine in that order. The reaction mixture was stirred for 2 hr at room temperature.

After the reaction was complete (LC-MS and TLC), the intermediate A21a was purified by prep-HPLC (228 mg).

### Preparation of Compound 21

| | Chemicals/Reagents & | | | | |
|---|---|---|---|---|---|
| S. No. | Solvents | MW | mmol | Eq. | Amts |
| 1 | A21a | | | | 228 mg |
| 2 | 4M HCl in MeOH | | | | 4 mL |

Cold HCl solution was added to the residue of A21a. The reaction mixture was stirred for overnight at room temperature.

After the reaction was complete (LC-MS), the mixture was concentrated. It was further purified on prep-HPLC to afford the final compound 21 (206 mg). Compound 21 was characterized by ¹H NMR, LC-MS and HPLC. ¹H NMR (D₂O), δ: 7.34-7.49 (m, 5H), 5.04 (s, 1H), 3.86-3.98 (m, 2H), 3.58-3.72 (m, 2H), 3.54 (t, J=6.22 Hz, 2H), 3.14-3.39 (m, 4H), 2.92-3.07 (m, 2H). LC-MS, (M+1), 264. HPLC (>95%, retention time, 0.98 min).

Preparation of lead analogs of a number of compounds of the present application is illustrated in the general Scheme 2 below:

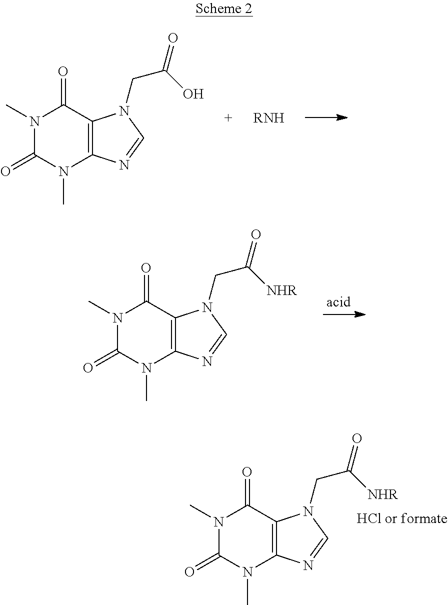

In such a synthesis strategy, the coupling agent can be HATU or HBTU. The acid used to remove a protection group such as Boc can be 4 M HCl in MeOH or 4M HCl in water.

Preparation of additional compounds of the present application can be illustrated in the general Scheme 3 below:

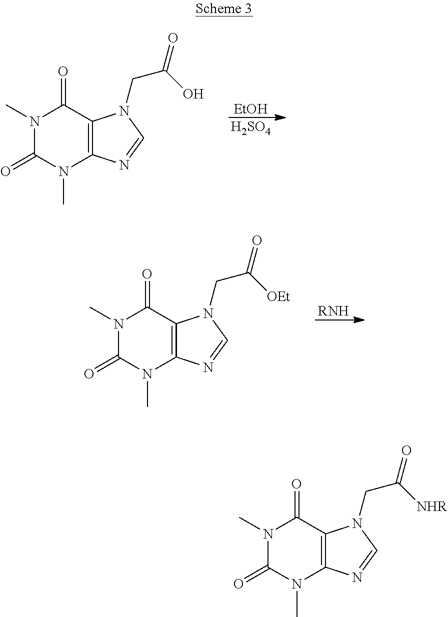

In this synthesis, the starting material acid is first converted to an ester. Then the ester is reacted with an amine to afford an amide compound. The amide compound may undergo further transformation, such as, reductive amination to afford the final compound.

### Example 22

### Preparation of Compound 22

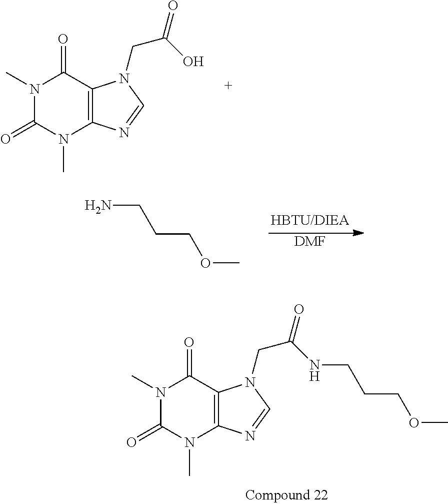

| | Chemicals/Reagents & | | | | |
|---|---|---|---|---|---|
| S. No. | Solvents | MW | mmol | Eq. | Amts |
| 1 | acid | 238.2 | 1 | 1.0 | 238 mg |
| 2 | DCM (anhydrous) | | | | 5 mL |
| 3 | DIEA, d = 0.742 | 129.25 | 2.0 | 2.0 | 0.35 mL |
| 4 | HBTU | 379.25 | 1 | 1.0 | 379 mg |
| 5 | Amine, d = 0.871 | 89.14 | 1 | 1.0 | 0.1 mL |

To a solution of acid in DMF were added HBTU, DIEA, and amine in that order. The reaction mixture was stirred over night at room temperature.

After the reaction was complete (LC-MS and TLC), brine and saturated sodium bicarbonate were added to quench the reaction. 10% MeOH in DCM was used to extract the aqueous layer (3×). The DCM layer was separated, combined and washed with brine, dried with anhydrous Na₂SO₄, filtered, and concentrated. The Compound 22 was obtained after silica gel column chromatography as an off-white solid (138 mg). Compound 22 was characterized by ¹H NMR, LC-MS and HPLC. ¹H NMR (D₂O), δ: 7.90 (s, 2H), 5.00 (s, 2H), 3.46 (s, 3H), 3.40 (t, J=6.40 Hz, 2H), 3.19-3.28 (m, 8H), 1.65-1.75 (m, 2H). LC-MS, (M+1), 310. HPLC (>95%, retention time, 5.76 min).

### Example 23

### Preparation of Compound 23

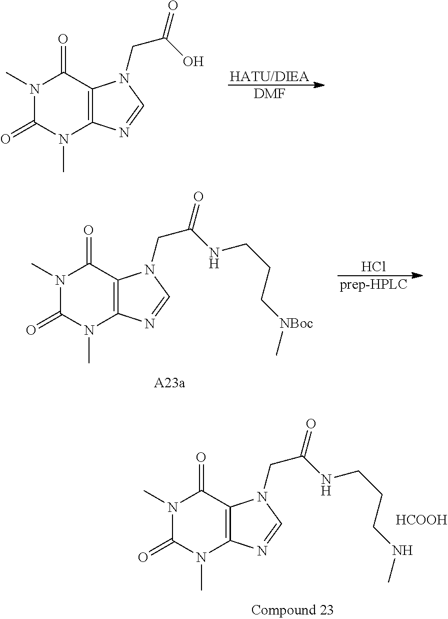

### Preparation of Intermediate A23a

| | Chemicals/Reagents & | | | | |
|---|---|---|---|---|---|
| S. No. | Solvents | MW | mmol | Eq. | Amts |
| 1 | acid | 238.2 | 1 | 1.0 | 238 mg |
| 2 | DMF (anhydrous) | | | | 5 mL |
| 3 | DIEA, d = 0.742 | 129.25 | 2.0 | 2.0 | 0.35 mL |
| 4 | HATU | 380.2 | 1 | 1.0 | 380 mg |
| 5 | Amine | 188.271 | 1 | 1.0 | 0.2 mL |

To a solution of acid in DMF were added HATU, DIEA, and amine in that order. The reaction mixture was stirred for 1 hr at room temperature.

After the reaction was complete (LC-MS and TLC), brine and saturated sodium bicarbonate were added to quench the reaction. 5% MeOH in DCM was used to extract the aqueous layer (3×). The DCM layer was separated, combined and washed with brine, dried with anhydrous Na₂SO₄, filtered, and concentrated. The compound was further purified by prep-HPLC (150 mg).

### Preparation of Compound 23

| S. No. | Chemicals/Reagents & Solvents | MW | mmol | Eq. | Amts |
|---|---|---|---|---|---|
| 1 | A23a | | | | 145 mg |
| 2 | 4M HCl in H₂O | | | | 4 mL |

Cold HCl solution was added to the residue of C9a. The reaction mixture was stirred for 3 hours at room temperature.

After the reaction was complete (LC-MS), the mixture was concentrated. It was further purified on prep-HPLC to afford the final compound 23 (73 mg). Compound 23 was characterized by ¹H NMR, LC-MS and HPLC. ¹H NMR (D₂O), δ: 7.89 (s, 1H), 4.99 (s, 2H), 3.45 (s, 3H), 3.25 (t, J=6.58 Hz, 2H), 3.22 (s, 3H), 2.96 (t, J=7.66 Hz, 2H), 2.60 (s, 3H), 1.76-1.86 (m, 2H). LC-MS, (M+1), 309. HPLC (>95%, retention time, 1.20 min).

### Example 24

### Preparation of Compound 24

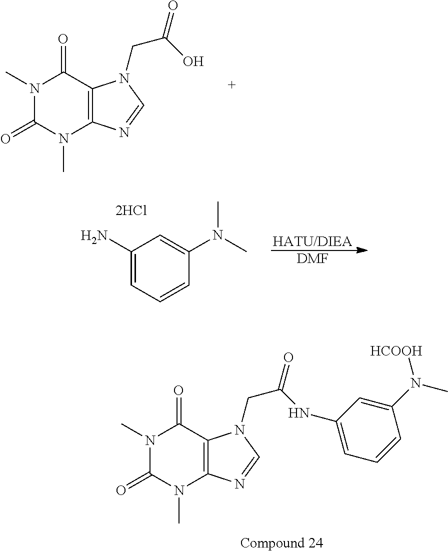

| S. No. | Chemicals/Reagents & Solvents | MW | mmol | Eq. | Amts |
|---|---|---|---|---|---|
| 1 | acid | 238.2 | 1 | 1.0 | 238 mg |
| 2 | DMF (anhydrous) | | | | 8 mL |
| 3 | DIEA, d = 0.742 | 129.25 | 2.0 | 2.0 | 0.7 mL |
| 4 | HATU | 380.2 | 1 | 1.0 | 380 mg |
| 5 | Aniline | 209.12 | 1 | 1.0 | 209 mg |

To a solution of acid in DMF were added HATU, DIEA, and aniline in that order. The reaction mixture was stirred for 2 hr at room temperature.

After the reaction was complete (LC-MS and TLC), the compound was purified by prep-HPLC (250 mg). Compound 24 was characterized by ¹H NMR, LC-MS and HPLC. ¹H NMR (CDCl₃), δ: 9.25 (s, 1H), 7.77 (s, 1H), 7.15 (t, J=8.14 Hz, 1H), 7.05 (s, 1H), 6.74-6.80 (m, 1H), 6.46-6.53 (m, 1H), 4.95 (s, 2H), 3.61 (s, 3H), 3.46 (s, 3H), 2.93 (s, 6H), 1.59 (s, 3H). LC-MS, (M+1), 357. HPLC (>95%, retention time, 5.79 min)

### Example 25

### Preparation of Compound 25

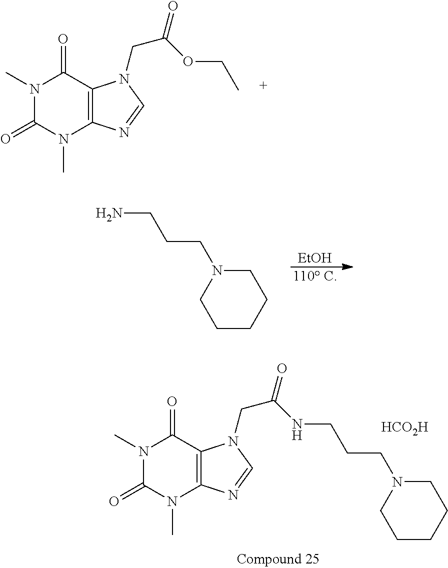 [SMILES: "EtOH" ; CCOC(=O)Cn1cnc2c1c(=O)n(C)c(=O)n2C ; NCCCN1CCCCC1 ; "110° C." ; Cn1c(=O)c2c(ncn2CC(=O)NCCCN2CCCCC2)n(C)c1=O ; "HCO2H"]

| S. No. | Chemicals/Reagents & Solvents | MW | mmol | Eq. | Amts |
|---|---|---|---|---|---|
| 1 | ester | 266.25 | 2.5 | 1 | 660 mg |
| 2 | 3-piperidin-1-yl-propylamine | 142.24 | 3.5 | 1.4 | 0.5 mL |
| 3 | EtOH | | | | 3 mL |

A mixture of ester (prepared by refluxing 18 g of Theophylline-7-acetic acid in 300 ml anhydrous EtOH with 1 mL concentrated H₂SO₄ as a catalyst) and 3-piperidin-1-yl-propylamine was suspended in anhydrous ethanol and sealed in a high pressure bottle. The reaction mixture was heated up to 110° C. with stirring for 2 hours.

LC-MS showed the completion of the reaction. 10 mL methanol was added and the solution was subjected to prep-HPLC separation (200 mg). Compound 25 was characterized by ¹H NMR, LC-MS and HPLC. ¹H NMR (D₂O), δ: 8.34 (s, 1H), 7.90 (s, 1H), 4.98 (s, 2H), 3.40-3.48 (m, 2H), 3.39 (s, 3H), 3.25 (t, J=6.48 Hz, 2H), 3.17 (s, 3H), 3.00-3.07 (m, 2H), 2.78-2.88 (m, 2H), 1.53-1.93 (m, 7H), 1.31-1.45 (m, 1H). LC-MS, (M+1), 363. HPLC (>95%, retention time, 1.91 min).

### Example 26

### Preparation of Compound 26

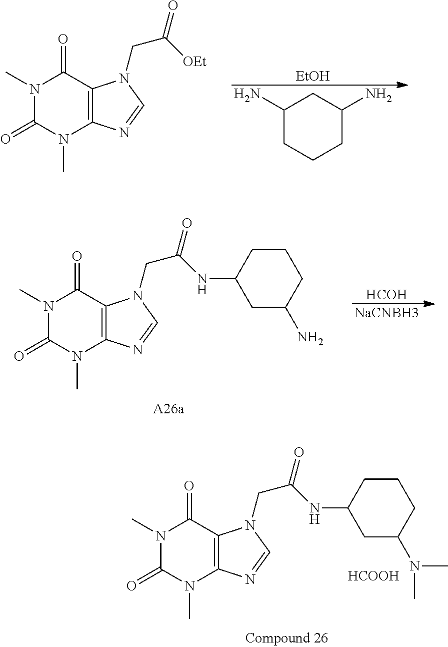

### Preparation of Intermediate A26a

| | Chemicals/Reagents & | | | | |
|---|---|---|---|---|---|
| S. No. | Solvents | MW | mmol | Eq. | Amts |
| 1 | Ester | 266.2 | 2.0 | 1.0 | 532 mg |
| 2 | EtOH, absolute | | | | 10 mL |
| 3 | 1,2 cyclohexane diamine, | 114.19 | 2.25 | 1.12 | 270 uL |
| | d = 0.95 | | | | |

The ester and the diamine were suspended in EtOH. The reaction mixture was refluxed for 2 hours.

The mixture was concentrated and purified on prep-HPLC. The compound was triturated with MeOH-Et₂O four times to remove trace amount of diamine (140 mg).

### Preparation of Compound 26

| | Chemicals/Reagents & | | | | |
|---|---|---|---|---|---|
| S. No. | Solvents | MW | mmol | Eq. | Amts |
| 1 | A26a | 344 | 0.4 | 1.0 | 136 mg |
| 2 | HCOH (aq., 37%) | | | | 4 mL |
| 3 | NaCNBH₃ | 62.84 | 0.82 | 2 | 51 mg |

A26a was dissolved in aqueous HCOH and NaCNBH₃ was added. The reaction mixture was stirred for 2 hours at RT.

The mixture was concentrated. It was purified on prep-HPLC to afford the Compound 26 (97 mg). Compound 26 was characterized by ¹H NMR, LC-MS and HPLC. ¹H NMR (D₂O), δ: 8.31 (s, 1H), 7.89 (s, 1H), 4.98 (s, 2H), 3.66-3.78 (m, 1H), 3.47 (s, 3H), 3.18-3.27 (m, 4H), 2.72-2.80 (m, 6H), 2.17-2.25 (m, 1H), 1.11-2.04 (m, 8H). LC-MS, (M+1), 363. HPLC (>95%, retention time, 1.95 min).

### Example 27

### Preparation of Compound 27

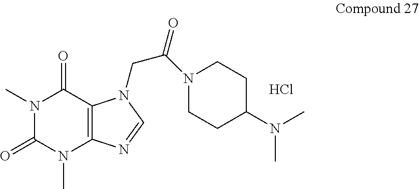

Compound 27 (180 mg) was prepared by the same method as Compound 26 using an appropriate starting material in place of 1,2-cyclohexane diamine. The only difference was that the reaction was carried out at 130° C. for 6 hours, and the final formic acid salt was converted to HCl salt by co evaporating with 50 mL of 1.25N HCl methanol solution three times. Compound 27 was characterized by ¹H NMR, LC-MS and HPLC. ¹H NMR (D₂O), δ: 7.85 (s, 1H), 5.19-5.38 (m, 3H), 4.41-4.50 (m, 1H), 4.00-4.08 (m, 1H), 3.46-3.53 (m, 1H), 3.45 (s, 3H), 3.18-3.28 (m, 4H), 2.80 (s, 6H), 2.68-2.78 (m, 1H), 2.04-2.21 (m, 2H), 1.73-1.86 (m, 1H), 1.53-1.66 (m, 1H). LC-MS, (M+1), 349. HPLC (>95%, retention time, 1.71 min).

### Example 28

### Preparation of Compound 28

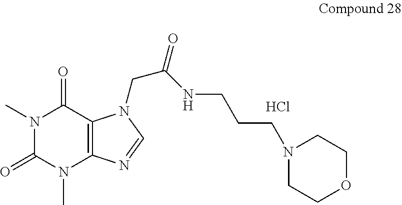 [SMILES: Cn1c(=O)c2c(ncn2CC(=O)NCCCN2CCOCC2)n(C)c1=O ; Cl]

Compound 28 (130 mg) was prepared by the same method as Compound 27 using a derivatized amine in place of 1,2-cyclohexane diamine, and the final formic acid salt was converted to HCl salt by co evaporating with 50 mL 1.25N HCl methanol solution three times. Compound 28 was characterized by ¹H NMR, LC-MS and HPLC. ¹H NMR (D₂O), δ: 7.89 (s, 1H), 4.99 (s, 2H), 3.97-4.05 (m, 2H), 3.64-3.75 (m, 2H), 3.40-3.47 (m, 5H), 3.16-3.29 (m, 7H), 3.03-3.16 (m, 4H), 1.83-1.94 (m, 2H). LC-MS, (M+1), 365. HPLC (>95%, retention time, 1.0 min).

### Example 29

### Preparation of Compound 29

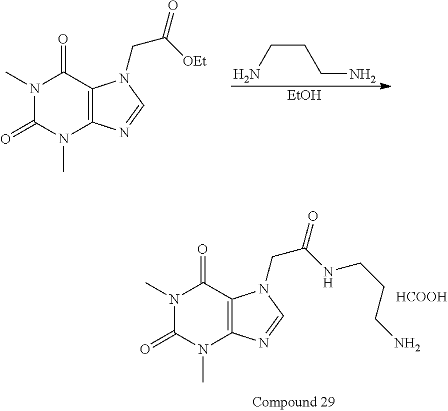 [SMILES: "EtOH" ; CCOC(=O)Cn1cnc2c1c(=O)n(C)c(=O)n2C ; NCCCN ; Cn1c(=O)c2c(ncn2CC(=O)NCCCN)n(C)c1=O ; "HCOOH"]

| S. No. | Chemicals/Reagents & Solvents | MW | mmol | Eq. | Amts |
|---|---|---|---|---|---|
| 1 | Ester | 266.2 | 1.0 | 1.0 | 266 mg |
| 2 | EtOH, absolute | | | | 5 mL |
| 3 | Diamine | 74.13 | 1.0 | 1.0 | 84 uL |

Ester and diamine were suspended in EtOH. The reaction mixture was refluxed for 2 hours.

After the reaction was complete (LC-MS), the mixture was concentrated. It was purified on prep-HPLC to afford Compound 29. The final compound was triturated with MeOH-Et2O four times to remove trace amount of diamine (155 mg). Compound 29 was characterized by ¹H NMR, LC-MS and HPLC. ¹H NMR (D₂O), δ: 8.33 (s, 1H), 7.87 (s, 1H), 4.99 (s, 2H), 3.44 (s, 3H), 3.24 (t, J=6.66 Hz, 2H), 3.20 (s, 3H), 2.91 (t, J=7.62 Hz, 2H), 1.74-1.82 (m, 2H). LC-MS, (M+1), 295. HPLC (>95%, retention time, 1.12 min).

### Example 30

### Preparation of Compound 30

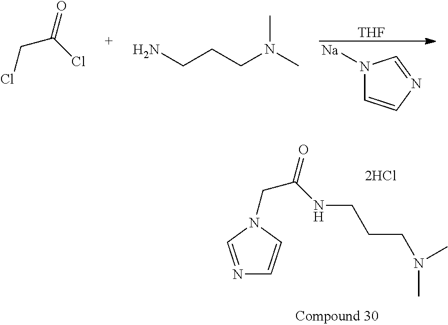

| S. No. | Chemicals/Reagents & Solvents | MW | mmol | Eq. | Amts |
|---|---|---|---|---|---|
| 1 | Chloroacetyl chloride | 112.94 | 10 | 1.0 | 0.8 mL |
| 2 | 3-(Dimethylamino)-1- | 102.18 | 10 | 1.0 | 1.2 mL |
| | propylamine | | | | |
| 3 | imidazole | 68.08 | 10 | 1.0 | 680 mg |
| 4 | NaH (60%) | 24.00 | 10 | 1.0 | 400 mg |
| 5 | THF | | | | 40 mL |

To a solution of chloroacetyl chloride in THF was added 3-(dimethylamino)-1-propylamine at 0° C. with stirring for 20 minutes. LC-MS showed the formation of amide. To this solution, imidazole sodium salt in THF (prepared from imidazole and NaH in 10 mL THF) was added. The reaction was continued for 2 hours with stirring.

After the reaction was complete (LC-MS), THF was removed. The residue was dissolved in methanol and subjected to prep-HPLC separation. The final compound was still not pure at this stage. Then it was purified again by ISCO C18 reverse phase chromatography to give the pure final product. The product was converted to HCl salt by evaporating together with HCl/methanol (1N) solution (100 mg). Compound 30 was characterized by ¹H NMR, LC-MS and HPLC. ¹H NMR (D₂O), δ: 8.71 (s, 1H), 7.36-7.48 (m, 2H), 5.02 (s, 2H), 3.25 (t, J=6.80 Hz, 2H), 3.04-3.10 (m, 2H), 2.78 (s, 6H), 1.83-1.92 (m, 2H). LC-MS, (M+1), 211. HPLC (>95%, retention time, 1.01 min).

### Example 31

### Preparation of Compound 31

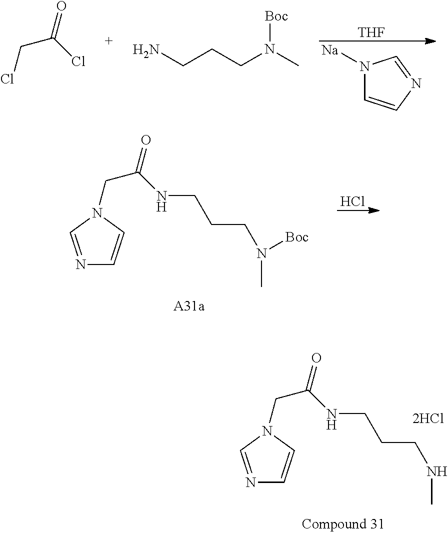

### Preparation of Intermediate A31a

Intermediate A31a (250 mg) was prepared similarly to Compound 30.

### Preparation of Compound 31

| S. No. | Chemicals/Reagents & Solvents | MW | mmol | Eq. | Amts |
|---|---|---|---|---|---|
| 1 | A31a | 296.37 | | | 250 mg |
| 2 | Methanol | | | | 50 mL |
| 3 | 12N HCl | | | | 25 mL |

Intermediate A31a was dissolved in methanol and HCl aqueous solution was added and the reaction was stirred for 2 hours.

After the reaction was complete (LC-MS), the mixture was concentrated. The residue was dissolved in water and subjected to prep-HPLC separation. After removal of solvent, the formic acid salt was converted to HCl salt by coevaporating with 50 mL 1.25 HCl methanol solution three times (200 mg). Compound 31 was characterized by ¹H NMR, LC-MS and HPLC. ¹H NMR (D₂O), δ: 8.70 (s, 1H), 7.33-7.46 (m, 2H), 5.01 (s, 2H), 3.25 (t, J=6.84 Hz, 2H) 2.95 (t, J=7.76 Hz, 2H), 2.60 (s, 3H), 1.77-1.87 (m, 2H). LC-MS, (M+1), 197. HPLC (>95%, retention time, 0.99 min).

### Example 32

### Preparation of enantiomerically pure 2-amino-3-methyl-N-(2-morpholino-ethyl)-pentanamide

2-amino-3-methyl-N-(2-morpholinoethyl)-pentanamide can be prepared by a method shown in Scheme 4 below. First, 2-aminoethanol (Compound 1E) is transformed to its derivative with a leaving group (Compound 2E). Examples of the leaving group include halides and alkoxy or other activated hydroxyl group. Second, Compound 2E reacts with morpholine at a neutral or basic condition to yield 2-morpholinoethanamine (Compound 3E). The aforementioned two steps may also be performed continuously as one step with Compound 2E being generated in situ. For example, Compound 3E can be prepared from Compound 1E directly through a Mitsunobu reaction wherein the hydroxyl group of Compound 1E is activated by diethyl azodicarboxylate (DEAD) before morpholine is added. The final product, 2-amino-3-methyl-N-(2-morpholinoethyl)-pentanamide (Compound 5E), can be obtained by coupling 2-morpholinoethanamine with 2-amino-3-methylpentanoic acid (Compound 4E) via a peptide coupling agent. Examples of the peptide coupling agent include 1,1′-carbonyldiimidazole (CDI), hydroxybenzo triazole (HOBT), 1,3-dicyclohexylcarbodiimide (DCC), 1-hydroxybenzo-7-azatriazole (HOAt), and the like.

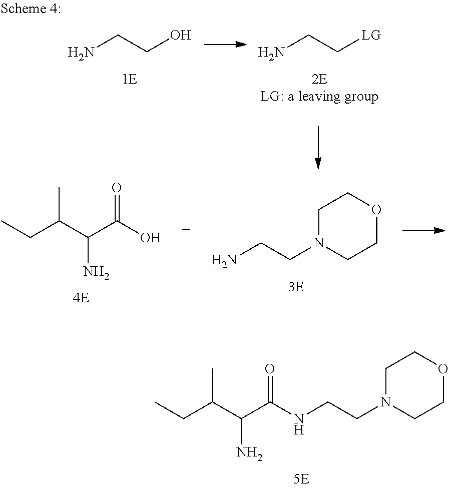

A chiral 2-amino-3-methyl-N-(2-morpholinoethyl)-pentanamide (Compound 5E) can be obtained by using the corresponding chiral 2-amino-3-methylpentanoic acid (Compound 4E) in the above coupling step. For example, (2S,3S)-2-amino-3-methyl-N-(2-morpholinoethyl)-pentanamide; (2R,3R)-2-amino-3-methyl-N-(2-morpholinoethyl)-pentanamide; (2R,3S)-2-amino-3-methyl-N-(2-morpholinoethyl)-pentanamide; and (2S,3R)-2-amino-3-methyl-N-(2-morpholinoethyl)-pentanamide can be obtained by using (2S,3S)-2-amino-3-methylpentanoic acid, i.e., L-isoleucine; (2R,3R)-2-amino-3-methylpentanoic acid, i.e., D-isoleucine; (2R,3S)-2-amino-3-methylpentanoic acid, i.e., D-alloisoleucine; and (2S,3R)-2-amino-3-methylpentanoic acid, i.e., L-alloisoleucine, respectively.

The chiral purity, also known as, enantiomeric excess or EE, of a chiral Compound 5E can be determined by any method known to one skilled in the art. For example, a chiral Compound 5E can be hydrolyzed to Compound 3E and the corresponding chiral Compound 4E. Then, the chiral Compound 4E obtained through hydrolysis can be compared with a standard chiral sample of Compound 4E to determine the chiral purity of the chiral Compound 5E. The determination can be conducted by using a chiral HPLC.

Each of the isomers demonstrated potent neurotrophic activity, according to the measurement of activity relative to BDNF, the details of which are disclosed below.

### Example 33

### Measurement of Activity Relative to BDNF

Compounds of the present application were tested for their ability to prevent the degeneration of hippocampal neurons as described in Massa et al J Neurosci. (2006) 26(20):5288-300. In brief, hippocampal neurons were isolated from embryological day 16 mice and seeded in 96-well tissue culture plates under conditions in which they degenerated in the absence of neurotrophin receptor ligands. Neuronal degeneration was assessed using morphological criteria 48 hours following cell seeding. The neurotrophins brain-derived neurotrophic factor (BDNF) and nerve growth factor (NGF) served as positive controls. The maximum cell death preventing activity of BDNF is defined as 100% neurotrophic activity. The efficacy of NGF is 80% of that of BDNF. The neurotrophic activity of the test compounds at each applied concentration was quantitated in terms of a percentage of the maximum BDNF-supported survival level. In the presence of culture medium (CM) and the absence of BDNF or compounds, survival is approximately 40% of the BDNF maximum effect and this is regarded as baseline survival. For each compound, dose-response curves were generated and the EC₅₀ and maximum survival percentage are derived. The compounds prepared and characterized as disclosed herein showed an EC₅₀ between about 1 nM and about 25 nM as well as a maximum efficacy between about 20% and about 100% of that of BDNF.

### Materials and Methods for Examples 34-40

Computational Studies

Computational studies were performed using the Accelrys Catalyst® and InsightII systems obtained from Accelerys (San Diego, Calif., United States of America).

Antibodies and Proteins

Polyclonal rabbit anti-NGF antibody was obtained from Chemicon (Temecula, Calif., United States of America). Monoclonal anti-phospho-ERK^{T202/Y204}, polyclonal anti-ERK42/44, monoclonal anti-phospho-AKT^{S473}, polyclonal anti-AKT, polyclonal anti-phospho-NFκB-p65(Ser⁵⁶³), and site-specific polyclonal anti-Trk^{Y490} were obtained from Cell Signaling Technology, Inc. (Beverly, Mass., United States of America). Monoclonal anti-NFκB-p65 was obtained from Santa Cruz Biotechnology, Inc. (Santa Cruz, Calif., United States of America). Monoclonal anti-actin was obtained from Sigma-Aldrich Corp. (St. Louis, Mo., United States of America). Polyclonal TrkA and TrkB antibodies were obtained from Upstate USA, Inc. (Charlottesville, Va., United States of America). Anti-pan-Trk 1087 and 1088 were previously characterized (Zhou, Holtzman, D. M., Weiner, R. I., Mobley, W. C. (1994) Proc Natl Acad Sci USA 91, 3824) and obtained from Dr. William C. Mobley (Stanford University, California, United States of America). p75^{NTR} polyclonal rabbit antibodies 9651 (Huber, L. J., Chao, M. V. (1995) Dev Bio 167, 227-238) and 9650 raised against the neurotrophin-binding region (residues 43-161, cysteine repeat regions II, III, and IV) of the extracellular domain of recombinant p75^{NTR} were provided by Dr. Moses Chao (Skirball Inst., NYU, New York, United States of America). Recombinant human NGF was obtained from Invitrogen (Carlsbad, California, United States of America) and BDNF from Sigma-Aldrich (St. Louis, Mo., United States of America). P75^{NTR}/Fc and TrkA/Fc chimerae were obtained from R&D Systems (Minneapolis, Minn., United States of America). Furin resistant pro-NGF was prepared as previously described (Beattie, M. S. et al. (2002) Neuron 36, 375-386).

Neural Bioassays

Hippocampal neurons were prepared from E16-17 mouse embryos as previously described (Yang, T. et al. (2003) J Neurosci 23, 3353-3363). Low density cultures were initiated in poly-L-lysine coated A/2 plates by adding 25 μl of cell suspension (2000 neurons/well; 12,500 cells/cm²), 25 μl of DMEM containing 10% FBS, and different concentrations of recombinant BDNF, NGF, or p75-binding compounds to each well. For studies employing p75^{NTR+/+} and p75^{NTR-/-} neurons, mice carrying a mutation in exon 3 of the p75^{NTR} gene (Lee, K. F. et al. (1992) Cell 69, 737-749) were bred onto a B6 congenic background (>10 B6 backcrosses).

After 48 hours in culture, cell survival was assessed as previously described (Longo, F. M., Manthorpe, M., Xie, Y. M., Varon, S. (1997) J Neurosci Res 48, 1-17) using a combination of standard morphological criteria along with visual determination of whether a given cell converted MTT to its blue formazan product. The number of surviving neurons was determined by counting the total number of cells in each well that were both morphologically intact and filed with blue product (Longo F. M., Manthorpe, M., Xie, Y. M., Varon, S. (1997) J Neurosci Res 48, 1-17). For each neurotrophin or compound concentration, duplicate wells were counted and the resulting values averaged. Activity of each compound was confirmed by blinded counts. Counts were normalized to survival achieved with 25 ng/ml BDNF or to baseline survival. Fitting of dose-responsive curves was performed with Sigmaplot obtained from SYSTAT Software Inc. (Richmond, Calif., United States of America).

For signaling pathway inhibitor studies, LY294002, PD98059 (obtained from EMD Biosciences/Calbiochem, San Diego, Calif., United States of America), and SN50 (obtained from Alexis Corp., Lausen, Switzerland) were added to cultures at final concentrations of 25 μM, 50 μM, and 2.5 μg/ml respectively, concomitantly with BDNF, NGF, or p75-binding compounds. For antibody inhibition studies, p75^{NTR} antisera and control non-immune serum were used at a final dilution of 1:100 in the presence of BDNF, NGF, or p75-binding compounds. For all studies applying signaling inhibitors, p75^{NTR} antibodies or p75^{NTR-/-} neurons, survival was assessed at 48 hours.

Protein Extraction and Western Blot Analysis

For assays of Trk, AKT, NFκB, and ERK activation, hippocampal neurons derived from E16-17 mice were cultured in poly-L-lysine coated six-well plates (Corning, Inc., Corning, N.Y., United States of America) in DMEM containing 10% FBS, followed by incubation in serum-free DMEM for 2 hours before addition of neurotrophins or compounds. At the indicated time points, neurons were harvested in lysis buffer consisting of: 20 mM Tris, pH 8.0, 137 mM NaCl, 1% Igepal CA-630, 10% glycerol, 1 mM PMSF, 10 μg/ml aprotinin, 1 μg/ml leupeptin, 500 μM orthovanadate (Zhou, J., Valletta, J. S., Grimes, M. L., Mobley, W. C. (1995) J Neurochem 65, 1146-1156).

Lysates were centrifuged, the supernatant collected, and protein concentrations determined using the BCA Protein Assay Reagent obtained from Pierce (Rockford, Ill., United States of America). Western blots were performed as described previously (Yang, T. et al. (2003) J Neurosci 23, 3353-3363). Western blot signals were detected using the ECL Chemiluminescence System obtained by Amersham (Piscataway, N.J., United States of America) (Yang, T. et al. (2003) J Neurosci 23, 3353-3363).

NGF Displacement from p75^{NTR} and TrkA

NGF ELISA was performed as previously described (Longo, F. M. et al. (1999) J Neurosci Res 55, 230-237). Briefly, 96-well plates were incubated with 0.1 pmol (at 1 nM) of p75/Fc or TrkA/Fc recombinant protein obtained from R&D System (Minneapolis, Minn., United States of America) overnight at 4° C. followed by incubation with blocking buffer for 1 hour at room temperature. ProNGF at 100 ng/ml or different concentrations of NGF, and p75-binding compounds were diluted in sample buffer, added to the wells, and incubated for 6 hours with shaking at room temperature. Plates were then washed five times with Tris-buffered saline (TBS) containing 0.05% Tween-20 and incubated with anti-NGF rabbit polyclonal antibody overnight at 4° C. Following five washes with TBS, wells were incubated for 2.5 hours at room temperature with anti-rabbit IgG HRP conjugate and washed five times. 3,3′,5,5′-tetramethyl-benzidine substrate was added and the optical density measured at 450 nm

p75^{NTR} Antibody Competition

NIH3T3 fibroblasts expressing either null vector or p75^{NTR} (Huang, C. S. et al. (1999) J Biol Chem 274, 36707-36714) were obtained from Dr. William Mobley (Stanford University, California, United States of America). Cells were grown in monolayers, harvested in PBS with 2 mM EDTA, pelleted, and resuspended in ice-cold DMEM HEPES with 1 mg/ml BSA. 6-9×10⁶ cells from one confluent 6-well plate were used for each experimental point.

For binding analysis, p75^{NTR} antibody (1:100) was allowed to bind in the presence or absence of 100 nM p75-binding compounds for 90 minutes at 4° C. with gentle rotation, followed by four washes in PBS. The final cell pellet was resuspended in lysis buffer.

Western blots were performed as described above. To detect the presence of p75^{NTR} antibody, blots were probed with horseradish peroxidase-linked goat anti-rabbit IgG obtained from Amersham/Pharmacia Biotech (Piscataway, N.J., United States of America). Signals were detected by the ECL chemiluminescence system obtained from Amersham Biosciences (Piscataway, N.J., United States of America). To control for variation in protein loading, the blot was stripped and reprobed with β-actin monoclonal antibody obtained from Sigma (St. Louis, Mo., United States of America).

Oligodendrocyte Culture, Pro-Neurotrophin Treatment and Cell Death Assay

Cortical oligondendrocytes from rat pups were prepared as previously described (Yoon et al. (1998) J Neurosci 18, 3273-3281, Harrington, A. W., Kim, J. Y., Yoon, S. O. (2002) J Neurosci 22, 156-166). Cells were treated with recombinant, cleavage-resistant proNGF at 0.05 nM (2.8 ng/ml). Controls were treated with equivalent volumes of proNGF purification buffer containing 350 mM imidazole. 24 hours after treatment, the cells were fixed and processed for MBP and TUNEL staining as previously described (Beattie, M. S. et al. (2002) Neuron 36, 375-386). 200-250 cells were counted per well, for a minimum of 600 cells per experimental condition.

### Example 34

### Computational Modeling, Pharmacophore Generation, Virtual and Functional Screening

In order to generate a productive pharmacophore emulating a loop structure likely to interact with a receptor, it was hypothesized that (1) the degrees of freedom of the ligand peptide structure are restricted by its residence in the protein, and (2) there is little “induced fit” involving changes in loop structure at the targeted receptor subsite, or it is accommodated by flexibility of the small molecule ligand. When both of these conditions apply, they allow an interacting/activating small molecule conformation that interacts with the receptor in a manner similar to that of the native ligand.

Computational studies of active dimeric cyclic peptides mimicking NGF β-hairpin loops suggest that energetic and structural constraints would disallow simultaneous β-hairpin folding of both peptide subunits, implying that the peptides act in a monomeric fashion. Additionally, based on early virtual screening of 3D conformer libraries, the presence of many functionally dimeric non-peptide molecules having a molecular weight of less than 500D was unlikely.

Therefore, efforts were focused on locating compounds emulating a single loop 1 structure. Compounds were selected for screening in cell survival assays using the protocol outlined in FIG. 1. Computational studies suggested that in situ, the tethered loop 1 backbone and proximal portions of the side chain structure had restricted degrees of freedom, and an intermediate structure chosen from an ensemble of samples from loop molecular dynamics simulations was extracted and used to build a novel pharmocophoric model (FIG. 1a). Guidance for the placement of pharmacophore features was obtained from consideration of loop phylogeny, side chain chemistry, and the inventors' experience with synthetic active peptides.

As a first approximation, it was assumed that analogous loop 1 domains from neurotrophins of different species and different neurotrophin family members should bind similarly to p75^{NTR}. The primary structure of BDNF loop 1 diverged significantly from NGF and NT-3, and in efforts to reduce pharmacophore complexity, only the latter two were utilized. The propensity of histidine to act as a hydrogen bond donor (McDonald, I, Thornton, J. M., (1994) WWW Edition December 1994) suggested its use at position 34, while the K to R transition at position 32 suggested the use of a positively ionizable feature at that location (FIGS. 1b and 1c).

An average of 35 conformers of each of over 800,000 compounds were screened against the novel pharmacophore, yielding approximately 800 that fit with a calculated internal energy of less than 10 kcal/mol (FIG. 1d). This number was reduced to approximately 60 by visual inspection on the basis of likely steric compatibility with a hypothetical shallow receptor binding pocket, and maximal flexibility of the functional groups. 35 compounds were obtained initially, of which 23 were soluble in water.

In preliminary studies using a chicken DRG neuronal survival assay, 4 of 23 compounds tested showed significant activity. Further analysis was carried out using embryonic mouse hippocampal neurons. Under conditions of low density and lack of glial support, the survival of these neurons was dependent, in part, on the addition of neurotrophins to the cultures.

Screening of 23 previously tested compounds in these hippocampal cultures demonstrated activity of three of the compounds (Compounds (ii-iv)) identified as having activity using DRG cultures, and also identified fourth and fifth active compounds (Compounds (i) and (vii)) through DRG and hippocampal assays. The results corresponded to a 17% yield for the screening procedure. In further support for this method, in preliminary studies based on models of NGF loop 4 (LM14A), a high positive rate of identification of active compounds was achieved (3 positive of 8 compounds screened (37%)).

A regression analysis of NGF-p75^{NTR} binding in the presence of the p75-binding compounds was performed. The data was fit using the Nonlinear Regression module of Sigmaplot, to a modification of the Gaddum/Schild equation (adapted from Motulsky, H. J., and Christopoulos, A. (2003) A Practical Guide to Curve Fitting, 2^{nd} edn. (San Diego, Calif., GraphPad Software, Inc.):

$$Signal = \frac{top - bottom}{1 + {( \frac{{{10}^{lo gEC 50} [ 1 + ( \frac{[ C ]}{{10}^{lo {gA}_{2}}} ) ]}^{S}}{[ NGF ]} )}^{HillSlope}} + bottom$$

where, top=maximal signal; bottom=basal signal; [C]=compound concentration; S=Schild coefficient; HillSlope=Hill coefficient; [NGF]=NGF concentration, EC₅₀=concentration of NGF resulting in 50% maximal (top-bottom) binding; and A₂=concentration of compound resulting in a doubling of the EC₅₀ from the unshifted curve. Herein, the calculated Hill slope ranged from 1.0 to 1.6 and was generally not significantly different from 1.

### Example 35

### Compounds Promote Hippocampal Neuron Survival

High-throughput virtual screening based on neurotrophin loop 1 models and small-scale in vitro bioassays were used to identify chemically diverse compounds with potent neurotrophic activity (FIG. 1). Approximately 800,000 compounds were screened in silico to produce a high yield of 4 positives out of 23 compounds submitted to in vitro screening (17%).

In order to understand the mechanisms of action of the selected compounds and test the conjecture that they work via the targeted receptor, p75^{NTR}, the dose-dependent relationships of the survival-promoting activities of the p75-binding compounds compared to NGF and BDNF using embryonic hippocampal neurons in culture conditions in which NGF promotes neural survival were examined. In the cultures, neurotrophic activity was mediated by BDNF principally through TrkB and p75^{NTR}, and by NGF primarily through p75^{NTR}, as they express little TrkA (Brann, A. B., et al. (1999) J Neurosci 19, 8199-8206; Bui, N. T., et al. (2002) J Neurochem 81, 594-605).

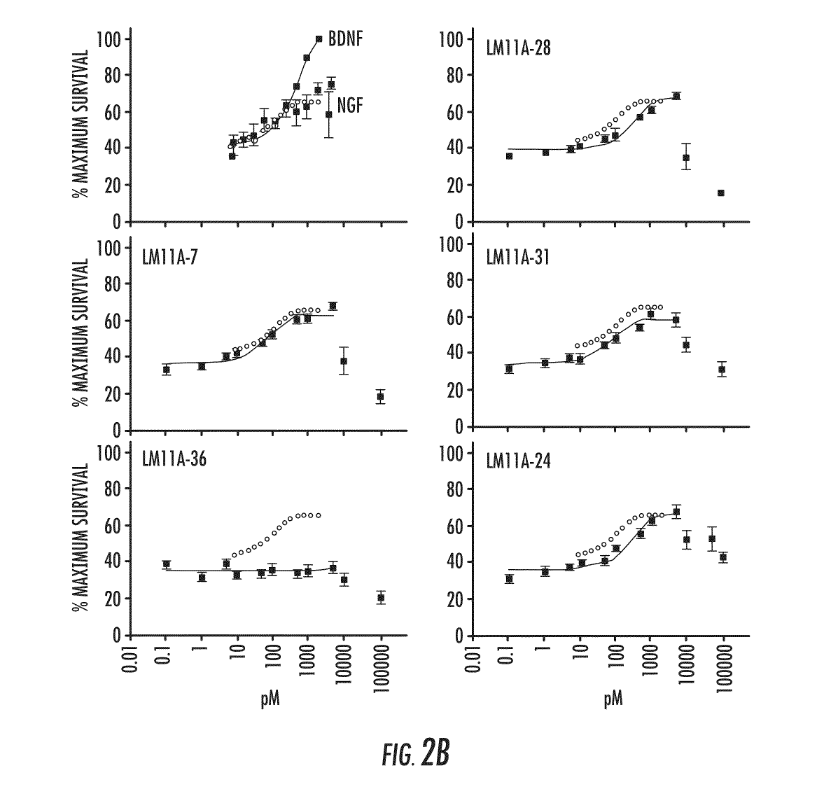

Addition of Compounds (i-iv) (FIG. 2a, structures) increased the number of GAP-43 positive neurite-bearing cells, consistent with increased neuronal survival (FIG. 2a, photomicrographs). Dose-response profiles of the active compounds (FIG. 2b) demonstrated EC₅₀ values in the range of 100-300 μM and intrinsic activities 80-100% of the NGF response. Independently synthesized preparations of Compound (iii) and Compound (iv) showed similar results. Compound (v), a compound structurally similar to Compound (iii), showed little or no neurotrophic activity. The structures of Compounds (i-iv) are illustrated in Table I.

For each compound, at concentrations greater than 5 nM, survival was reduced to baseline or below (FIG. 2b) as a result of mechanisms unrelated to p75^{NTR}, or from modulation of receptor multimer formation and survival signaling at higher receptor occupancies, even by a compound without neurotrophic activity (e.g., Compound (v)). Response curves similar to that of NGF in the hippocampal neuron system are consistent with activation of survival signaling through the NGF binding region of p75^{NTR}.

Of the four compounds initially identified, the two (Compound (iii), a derivative of caffeine, and Compound (iv), an amino acid derivative) were predicted to have the most “drug-like” character by the Lipinski criteria (Lipinski, C. A. (2000) J Pharm Toxicol Methods 44, 235-249) and blood-brain barrier calculations (Fu, X. C., Chen, C. X., Liang, W. Q., Yu, Q. S. (2001) Acta Pharmacol Sin 22, 663-668; Clark, D. E. (2002) J Pharm Sci 88, 815-821) were selected for more detailed mechanistic study. Compound (iv) was prioritized, as preliminary studies indicated that it exhibits significant oral uptake and blood-brain barrier penetration. The relatively inactive Compound (v) was chosen as a negative control due to its structural similarity to Compound (iii) (FIG. 2a).

### Example 36

### Compounds Interact with and Work Through p75^{NT} Receptors not Trk Receptors

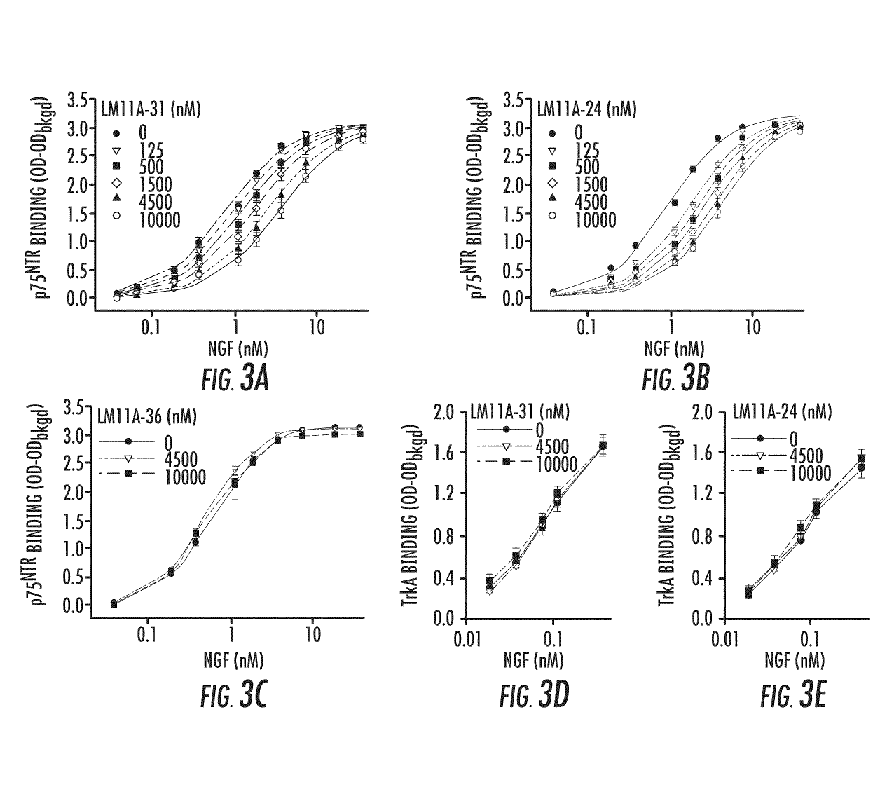

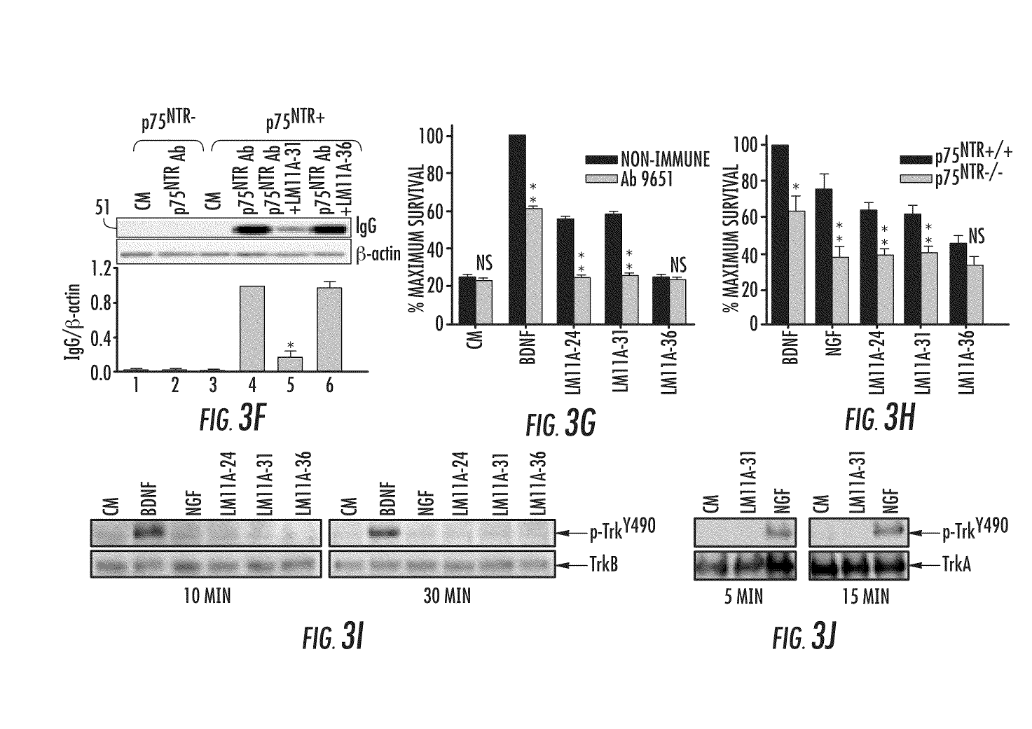

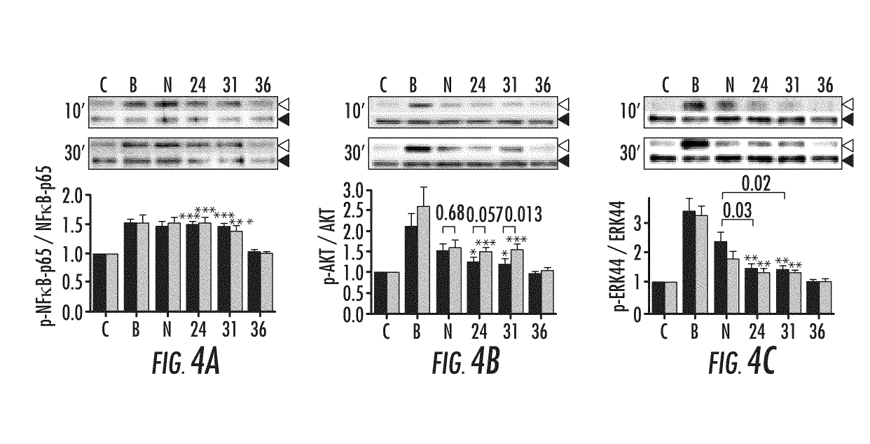

In order to assess the interactions of the p75-binding compounds with neurotrophin receptors, the effects of increasing concentrations of compounds on NGF binding to the recombinant chimeric proteins p75^{NTR}-Fc and TrkA-Fc were examined. In these experiments, Compound (iv) (FIG. 3a) and Compound (iii) (FIG. 3b), but not Compound (v) (FIG. 3c), shifted the NGF/p75^{NTR}-Fc biding curve significantly to the right. The inhibition of NGF binding caused by each active compound was reversed with increasing NGF concentration, consistent with a mechanism that is, at least in part, competitive in nature. When the data was fit to the Gaddum-Schild equation that describes ligand binding in the presence of an inhibitor (Motulsky, H. J., and Christopoulos, A. (2003) A Practical Guide to Curve Fitting, 2^{nd} edn. (San Diego, Calif., GraphPad Software, Inc.)), the resulting Schild coefficients were significantly less than 1.0 for both active compounds (Compound (iv), 0.58+/−0.04; Compound (iii), 0.26+/−0.01), suggesting a more complex model, e.g., multiple ligand-receptor binding sites (Lutz, M., and Kenakin, T. (1999) Quantitative Molecular Pharmacology and Informatics in Drug Discovery (Hoboken, N.J.: John Wiley & Sons); Neubig, R. R., Spedding, M, Kenakin, T., and Christopoulos, A. (2003) Pharmacol Rev 55, 597-606).

The results are consistent with models in which the effects of the compounds are due either to interaction with only a portion of the NGF binding surface of the receptor, and/or to allosteric effects indirectly affecting NGF binding. Gaddum-Schild analysis also yields a measure of potency known as A₂ (i.e., the concentration of compound that shifts the EC₅₀ twofold to the right) that can be equated with compound K_{D} when the Schild coefficient is 1. The A₂ values derived for Compound (iv) and Compound (iii) were 1192+/−1.2 and 31.6+/−1.3 nM, respectively. However, since the Schild coefficients are significantly different from 1, the true K_{D} values are unable to be determined.

In the case of Compound (iv), the EC₅₀ value for its biologic effect is approximately 150 pM, while its A₂ is nearly four orders of magnitude greater. Large differences between biologic potency of small molecules and binding estimated by ligand displacement are common (Lutz, M., and Kenakin, T. (1999) Quantitative Molecular Pharmacology and Informatics in Drug Discovery (Hoboken, N.J.: John Wiley & Sons)), and may have several causes, including: differences between receptor states in binding versus functional assays; post-receptor signal amplification, such that maximal biologic effects are seen at very low receptor occupancies; partial displacement of a multivalent ligand by a smaller antagonist; and that the compound works through a mechanism independent of the targeted receptor. The latter possibility was addressed using p75^{NTR} blocking antibody and p75^{NTR-/-} neurons to assess p75^{NTR} dependence. Additionally, the specificity of p75-binding compounds for p75^{NTR} is supported by the finding that the active compounds have no effect on NGF binding to TrkA (FIGS. 3d, 3e).

It was then determined that p75-binding compound activity is p75^{NTR}-dependent. Ab 9651, previously shown to block neurotrophic activity of NGF and NGF loop 1 peptide mimetics in mouse dorsal root ganglion neurons (Longo, F. M., Manthorpe, M., Xie, Y. M., Varon, S. (1997) J Neurosci Res 48, 1-17), partially blocked the neurotrophic activity of BDNF (FIG. 3g) and completely blocked the neurotrophic activity of Compound (iii) and Compound (iv), while non-immune antibody had no effect. Another independently-derived rabbit polyclonal anti-p75^{NTR} antibody (Ab 9650) gave virtually identical results, further corroborating the specificity of the p75^{NTR} blockade. Neither of the antibodies produced changes in baseline survival, suggesting that in these cultures the antibody preparations do not promote or inhibit survival. These results are consistent with BDNF acting through both TrkB and p75^{NTR} while NGF and p75-binding compounds act primarily through p75^{NTR}.

In addition, the response of p75^{NTR}-deficient (−/−) cells to neurotrophins and the p75-binding compounds were examined (FIG. 3h). Baseline survival under these culture conditions was the same in wild type and deficient cells, while p75^{NTR}-deficiency was associated with partial responsiveness to BDNF, and lack of response to NGF and the p75-binding compounds, a pattern similar to that found in the p75^{NTR}-antibody studies. Finally, treatment with 5 nM Compound (iii) or Compound (iv) along with 50 ng/ml NGF, concentrations of each which induce a maximal response, produced no additive effect on survival, further supporting the hypothesis that the p75-binding compounds act directly through binding to p75^{NTR}.

Despite the observation that Compound (iii) and Compound (iv) did not affect NGF-TrkA/Fc binding, the question remained whether the p75-binding compounds activate TrkB, the principal Trk on the hippocampal neurons, or the nominally expressed TrkA, as their primary mechanism for promoting survival. Also, ligand binding to p75^{NTR} might influence Trk activation. With these considerations, it was of interest to determine whether the p75-binding compounds promote Trk activation. Compound (iii) and Compound (iv) were assessed for the ability to activate Trk autophosphorylation, as indicated by Trk^{Y490} phosphorylation, a well-established marker of Trk activation. In hippocampal cultures, BDNF exposure resulted in robust Trk activation (FIG. 3i), while no activation was detected with NGF or the p75-binding compounds. The lack of signal with NGF confirms that these cultures produce little or no TrkA and supports the idea that the trophic effects of NGF are mediated principally by p75^{NTR}. In 3T3-TrkA cells, NGF exposure produced the expected TrkA autophosphorylation response, while the p75-binding compounds again showed no activity (FIG. 3j). These results suggested that activation of Trk receptors does not play a primary role in the promotion of cell survival by p75-binding compounds.

### Example 37

### Compounds Work Through p75^{NTR}

Together, the displacement of NGF from p75^{NTR} by the active compounds, but not with an inactive compound, the dependence of biologic function on the presence of an unoccluded p75^{NTR} lack of Trk interactions and activation and lack of additive effects between NGF and compound, strongly suggest that the p75-binding compounds directly interact with and work through p75^{NTR}.

This is consistent with the pharmacophoric model used herein to select the p75-binding compounds (FIG. 1). Given fitting to this model as the principal initial selection criterion, the identification of a high percentage of chemically diverse positives from a small group tested in vitro, and their similar actions in a variety of biochemical and biologic assays, the evidence suggests that the p75-binding compounds interact at a p75^{NTR} neurotrophin binding site rather than at other locations in the receptor.

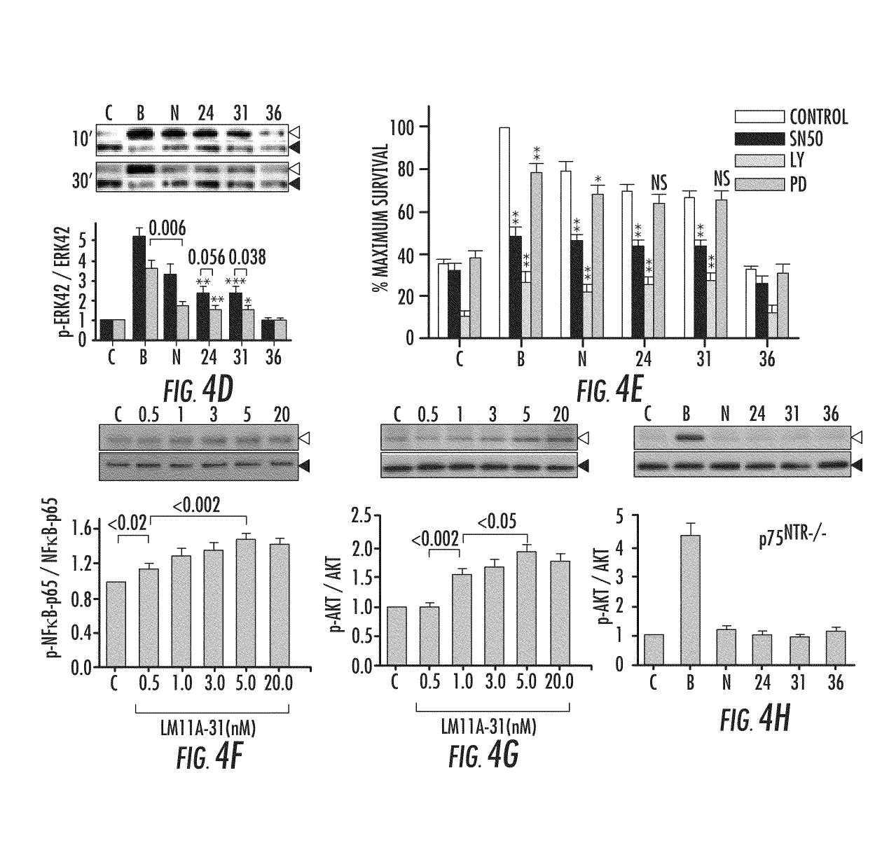

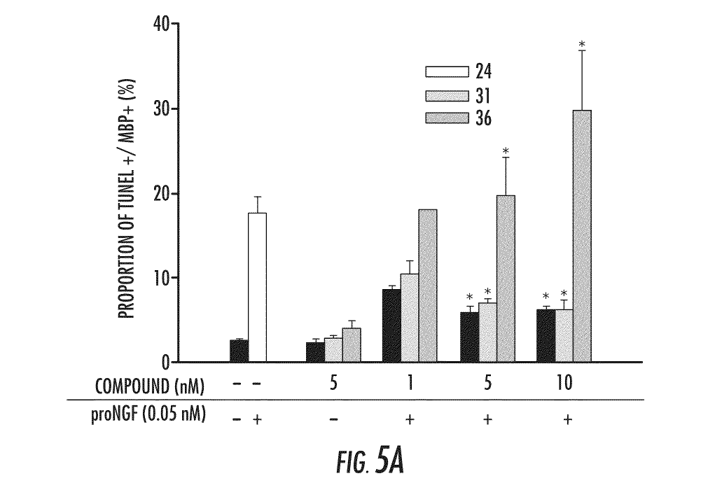

Pro-survival signaling associated with p75^{NTR} actions include activation of P13K and AKT (Roux, P. P., Bhakar, A. L., Kennedy, T. E., Barker, P. A. (2001) J Biol Chem 276, 23097-23104; Lachyankar, M. B., et al. (2003) J Neurosci Res 71, 157-172), NFκB (Mamidipudi, V., Li, X., Wooten, M. W. (2002) J Biol Chem 277, 28010-28018; Carter, B. D., et al. (1996) Science 272, 542-545; Gentry, J. J., Casaccia-Bonnefil, P., Carter, B. D. (2000) J Biol Chem 275, 7558-7565; Foehr, E. D., et al. (2003) J Neurosci Res 73, 7556-7563), and ERK (Lad, S. P., Neet, K. E. (2003) J Neurosci Res 73, 614-626). Each of these signaling intermediates has been shown to be capable of being independently regulated by Trk and p75^{NTR} through pathways with varying degrees of overlap and crosstalk, and with different kinetics. Treatment of hippocampal neurons with 20 nM Compound (iii) and Compound (iv) (a concentration in the plateau range for acute signaling activation) led to an approximately 1.5 fold increase in NFκB-p65 phosphorylation (FIG. 4a), indicative of activation of the NFκB pathway (Sakurai, H., Chiba, H., Miyoshi, H., Sugita, T., Toriumi, W. (1999) J Biol Chem 274, 30353-30356), similar in extent and time course to that induced by both neurotrophin proteins.

Compound (v) at the same concentration did not induce NFκB-p65 phosphorylation. Consistent with activation of NFκB by Compound (iii) and Compound (iv), a specific peptide inhibitor of NFκB translocation, SN50 (Lin, Y. Z., Yao, S. Y., Veach, R. A., Torgerson, T. R., Hawiger, J. (1995) J Biol Chem 270, 14255-14258), significantly reduced cell survival promoted by Compound (iii), Compound (iv) and the neurotrophins, with no effect on baseline, survival (FIG. 4e).

In studies of AKT activation, Compound (iii) and Compound (iv) at 20 nM showed similar degrees of activation to that found with NGF at 30 minutes, while BDNF induced a substantially greater response. In addition, the onset of activation stimulated by Compound (iii) and Compound (iv) was slower than that of NGF (FIG. 4b). Consistent with these findings, the P13 kinase inhibitor LY294002, which inhibits AKT activation, markedly decreased survival in all cases, including baseline survival under conditions of no treatment or exposure to Compound (v) (FIG. 4e).

Investigation of ERK signaling showed that ERK44 activation was induced to a greater extent by the neurotrophins than by the p75-binding compounds (FIG. 4c), which showed a significant but small response. ERK42 activation was more robust overall and greater with BDNF and NGF treatment than with the p75-binding compounds. There was a prominent loss of signal by 30 minutes but with greater persistence of the activated form following BDNF treatment (FIG. 4d). Consistent with the finding of greater ERK activation induced by BDNF compared to NGF and p75-binding compounds, the ERK inhibitor PD98059 significantly decreased BDNF-stimulated survival while it had a small but significant effect on NGF activity, and produced no significant decrease in survival promoted by either Compound (iii) or Compound (iv) (FIG. 4e).

These observations suggest that unlike NFκB and P13K, ERK activation is not a significant factor in the promotion of survival by the p75-binding compounds. The difference likely relates to the lower levels of ERK activation observed with the p75-binding compounds relative to the protein ligands. P13K activation can promote survival through pathways involving and not involving AKT (Zhang, Y., et al. (2003) J Neurosci 23, 7385-7394), and so the essential mechanisms downstream of P13K in this system remain to be determined. The more robust activation of AKT and ERKs by BDNF likely represents the influence of TrkB.

To further examine the relationship between compound-mediated AKT and NFκB activation, and neural survival, compound dose-activation studies were performed (FIGS. 4f, 4g). The results demonstrate that Compound (iv) induces activation of both AKT and NFκB over a concentration range of 0.5 nM to 3 nM, which is similar to that required for promotion of survival, and concordant with a role for these signaling mechanisms in mediating compound-induced survival.

Further, it was determined that NGF and compound activation of AKT signaling was completely absent in cultures of p75^{NTR-/-} neurons (FIG. 4h), consistent with the hypothesis that NGF and p75-binding compounds activate AKT survival signaling through p75^{NTR} Together with the evidence for p75^{NTR} dependence of compound-induced survival (FIGS. 3g, 3h), these findings suggest that p75-binding compounds induce survival of hippocampal neurons in culture, at least in part, through interactions with p75^{NTR} that produce activation of survival-promoting signaling pathways involving AKT and NFκB.

### Example 38

### Compound (iii) and Compound (iv) do not Promote Cell Death of Mature Oligodendrocytes, but Inhibit Pro-NGF-Induced Death

Though NGF and the p75-binding compounds promoted cell survival in the hippocampal cultures used in the studies herein, liganding of p75^{NTR} by mature NGF or proNGF, has been associated with cell death rather than promotion of survival in certain cell types (Lee, R., Kennan, P, Teng, K. K., Hempstead, B. L. (2001) Science 294, 1945-1948; Casaccia-Bonnefil, P., Carter, B. D., Dobrowsky, R. T., Chao, M. V. (1996) Nature 386, 716-719). To determine whether the p75-binding compounds disclosed herein promote survival or cause death in systems in which neurotrophins promote cell death, the survival of mature oligodendrocytes treated with p75-binding compounds and proNGF was examined.

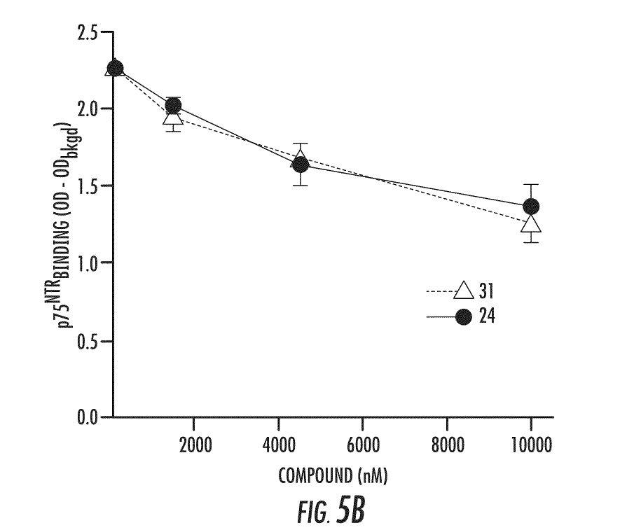

Mature oligodendrocytes express p75^{NTR} but not TrkA, and undergo apoptotic death on treatment with NGF or proNGF (Beattie, M. S., et al. (2002) Neuron 36, 375-386; Casaccia-Bonnefil, P., Carter, B. D., Dobrowsky, R. T., Chao, M. V. (1996) Nature 386, 716-719; Yoon, S. O., Casaccia-Bonnefil, P., Carter, B., Chao, M. V. (1998) J Neurosci 18, 3273-3281). Unlike NGF or proNGF, Compound (iii), Compound (iv) and Compound (iv) alone did not promote cell death (FIG. 5a). In addition, pro-NGF-induced cell death was significantly inhibited by Compound (iii) and Compound (iv) over a concentration range of 1 to 10 nM, but not by Compound (v), which appeared to decrease survival at 10 nM (FIG. 5a).

In order to determine whether p75-binding compounds block proNGF binding to p75^{NTR}, proNGF binding to p75^{NTR} was assessed over a concentration range of 1500 nM to 10,000 nM. Compound (iii) and Compound (iv) inhibited proNGF binding equally, up to an approximately 30% decrement at the highest concentration (FIG. 5b). The high concentration required to inhibit pro-NGF binding compared to those blocking proNGF-induced death suggest the possibilities that: 1) in the cell-based assay, with native receptor conformation in the presence of co-receptors (e.g., sortilin), the proNGF-p75^{NTR} interaction may be more susceptible than in the in vitro assay to disruption by the p75-binding compounds; 2) at low concentrations, the compounds qualitatively alter proNGF binding to decrease the induction of cell death, but do not decrease the total amount of proNGF binding to decrease the induction of cell death but do not decrease the total amount of proNGF binding; or 3) that the compounds induce preferential activation of pro-survival signaling by p75^{NTR} without affecting proNGF binding. Preferential survival pathway activation could result from differences in the way the compounds modulate receptor structure, as well as lack of binding to co-receptors expressed by oligodendrocytes, such as sortilin. Indeed, prior studies suggest that engagement of both sortilin and p75^{NTR} by proNGF promotes efficient ligand binding, receptor complex activation and apoptotic actions (Nykjaer, A., Willnow, T. E., and Petersen, C. M. (2005) Curr Opin Neurobiol 15, 49-57).

### Example 39

### Compound (iii) Blocks AD-Induced Neural Degeneration

Using previously well established protocols, Aβ was preincubated for 3 days in water to allow formation of oligomers. E17 hippocampal neurons were incubated for 5 days to allow for maturation prior to addition of Aβ with test compounds. Mature neurons demonstrate high Aβ vulnerability.

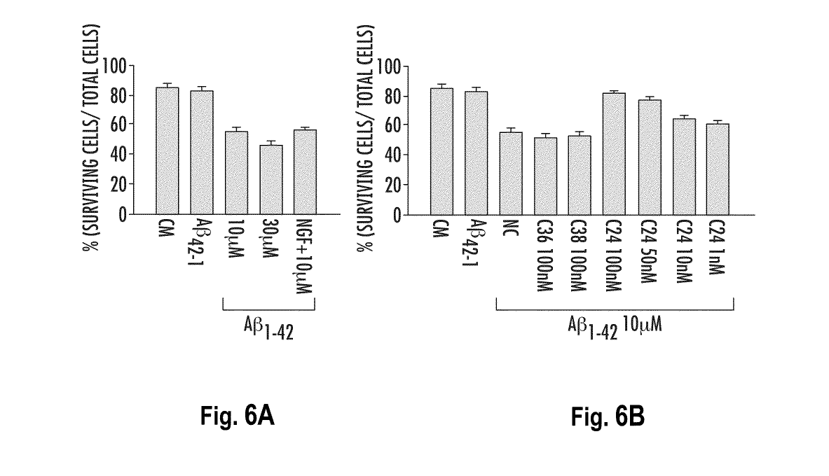

Addition of Aβ₄₂₋₁ (30 μM) as a negative control caused no cell death. Addition of Aβ₁₋₄₂ at 10 μM or 30 uM caused an approximate 40% loss of neurons after a 3 day exposure (FIG. 6a). The results are similar to in vitro Aβ-induced death levels reported previously (Michaelis, M. L., et al. (2006) J Pharm Exp Ther 312:659-668). Addition of NGF (100 pg/ml) fails to protect against Aβ₁₋₄₂, a finding previously reported (Yankner, B. A., et al. (1990) PNAS 87:9020-9023).

Addition of Aβ₁₋₄₂ in the absence of compound (NC) resulted in a 40% loss of neurons (FIG. 6b). The presence of inactive Compound (v) and Compound (vi) failed to block Aβ₁₋₄₂ toxicity. Addition of Compound (iii), however, blocks Aβ-induced death with a dose-response effect and an EC₅₀ of approximately 10 nM. Data are expressed as percentage surviving cells over total cells present in a given measurement area. Mean+/−SE is shown with at least 20 areas measured per condition over multiple bioassays. The ability of Compound (iv) to entirely block Aβ-induced degeneration at low nanomolar concentrations, its favorable molecular weight (less than 500), and a favorable Lipinski score indicate that it is a high priority lead compound for preclinical development in in vivo AD models. Compound (iv) has also been shown to block Aβ-induced degeneration of cortical and septal neurons.

### Example 40

### Compound (iv) Prevents Hair Loss in Middle Aged Mice

In a three-month toxicology trial, the presence of age-related hair loss was demonstrated in 3 of 5 vehicle-treated mice and in 0 of 5 Compound (iv)-treated male mice. In a follow up study, 4 of 10 vehicle-treated and 0 of 9 Compound (iv)-treated middle-aged male mice at the 2-month time point demonstrated hair loss.

Taken together, these studies indicate that 7 of 15 vehicle-treated, and 0 of 14 Compound (iv)-treated mice demonstrate hair loss. The resulting p value is 0.001 (Fisher's Exact test), supporting the presence of a significant effect in the preclinical studies. This data indicates p75^{NTR} regulates the death of hair follicle cells and thereby the process of hair loss (known as catagen). These findings, for the first time, indicate the efficacy of administering small molecule compounds targeting p75^{NTR} for the prevention of hair loss occurring during aging or in pathological states, such as alopecia areata.

### Summary of the Examples 34-40

In targeting one member of a group of receptors that interact with a given ligand, activation of a subset of receptor-mediated effects which can or can not be naturally occurring was anticipated. Such differences in signaling patterns, including minimizing activation of Trks, will likely prove clinically useful. For example, the compounds disclosed in the Examples can promote survival under conditions where neurotrophins promote death, and can be less likely to induce excessive sympathetic fiber sprouting and upregulation of pain transmission occurring, likely via Trk signaling, with neurotrophin treatments (Walsh, G. S., Krol, K. M., Kawaja, M. D. (1999) J Neurosci 19, 258-273).

### REFERENCES

Appel, S. H. (1981) Ann Neurol 10, 499.
Beattie, M. S. et al. (2002) Neuron 36, 375-386.
Braun, A. B., et al. (1999) J Neurosci 19, 8199-8206.
Bui, N. T., et al. (2002) J Neurochem 81, 594-605.
Carter, B. D., et al. (2002) Science 272, 542-545.
Casaccia-Bonnefil, P., Carter, B. D., Dobrowsky, R. T., Chao, M. V. (1996) Nature 386, 716-719.
Clark, D. E. (2002) J Pharm Sci 88, 815-821.
Fahnestock, M., Michalski, B., Xu, B., Coughlin, M. D. (2001) Mol Cell Neurosci 18, 210-220.
Foehr, E. D., et al. (2003) J Neurosci Res 73, 7556-7563.
Freireich et al., (1966) Cancer Chemother Rep. 50, 219-244.
Fu, X. C., Chen, C. X., Liang, W. Q., Yu, Q. S. (2001) Acta Pharmacol Sin 22, 663-668.
Gentry, J. J., Casaccia-Bonnefil, P., Carter, B. D. (2000) J Biol Chem 275, 7558-7565.
Harrington, A. W. et al. (2004) Proc Natl Acad Sci USA 101, 6226-6230.
Harrington, A. W, Kim, J. Y., Yoon, S. O. (2002) J Neurosci 22, 156-166.
He, X. L., Garcia, K. C. (2004) Science 304, 870-875.
Huang, C. S. et al. (1994) J Biol Chem 274, 36707-36714.
Huber, L. J., Chao, M. V. (1995) Dev Bio 167, 227-238.
Lachyankar, M. B., et al. (2003) J Neurosci Res 71, 157-172.
Lad, S. P., Neet, K. E. (2003) J Neurosci Res 73, 614-626.
Lee, K. F. et al. (1992) Cell 69, 737-749.
Lee, R., Kermani, P., Teng, K. K., Hempstead, B. L. (2001) Science 294, 1945-1948.
Lin, Y. Z., Yao, S. Y., Veach, R. A., Torgerson, T. R., Hawiger, J. (1995) J Biol Chem 270, 14255-14258.
Lipinski, C. A. (2000) J Pharm Toxicol Methods 44, 235-249.
Longo, F. M. et al. (1999) J Neurosci Res 55, 230-237.
Longo, F. M., Manthorpe, M., Xie, Y. M., Varon, S. (1997) J Neurosci Res 48, 1-17.
Lutz, M., and Kenakin, T. (1999) Quantitative Molecular Pharmacology and Informatics in Drug Discovery (Hoboken, N. J.: John Wiley & Sons).
Maliartchouk, S., Debeir, T., and Beglova, N. Cuello, A. C., Gehring, K, and Saragovi, H. U. (2000) J Biol Chem 275, 9946-9956.
Mamidipudi, V., Li, X., Wooten, M. W. (2002) J Biol Chem 277, 28010-28018.
McDonald, I, Thornton, J. M., (1994) WWW Edition December 1994.
Michaelis, M. L., Ansar, S., Chen, Y., Reiff, E. R., Seyb, K. I., Himes, R. H., Audus, K. L., and Georg, G. I. (2006) J Pharm Exp Ther 312:659-668.
Motulsky, H. J., and Christopoulos, A. (2003) A Practical Guide to Curve Fitting, 2^{nd} edn. (San Diego, Calif., GraphPad Software, Inc.).
Neubig, R. R., Spedding, M, Kenakin, T., and Christopoulos, A. (2003) Pharmacol Rev 55, 597-606.
Nykjaer, A. et al., (2004) Nature 427, 843-848.
Nykjaer, A., Willnow, T. E., and Petersen, C. M. (2005) Curr Opin Neurobiol 15, 49-57.
Partridge, W. M. (2002) Adv Exp Med Bio 513, 397-430).
Podulso, J. F., Curran, G. L. (1996) Brain Res Mol Brain Res 36, 280-286.
Remington: The Science and Practice of Pharmacy, A. Gennaro, ed., 20th edition, Lippincott, Williams & Wilkins, Philadelphia, Pa.
Remington's Pharmaceutical Sciences, Mack Pub. Co., Easton, Pa., 1980.
Roux, P. P., Bhakar, A. L., Kennedy, T. E., Barker, P. A. (2001) J Biol Chem 276, 23097-23104.
Sakurai, H., Chiba, H., Miyoshi, H., Sugita, T., Toriumi, W. (1999) J Biol Chem 274, 30353-30356.
Salehi, A. H., et al. (2000) Neuron 27, 279-288.
Saltzman, W. M., Mak, M. W., Mahoney, M. J., Duenas, E. T., Cleland, J. L. (1999) Pharm Res 16, 232-240.
Walsh, G. S., Krol, K. M., Kawaja, M. D. (1999) J Neurosci 19, 258-273.
Wang, J. J., Rabizadeh, S., Tasinato, A., Sperandio, S., Ye, X., Green, M., Assa-Munt, N., Spencer, D., and Bredesen, D. E. (2000) J Neurosci Res 60, 587-593.
Yang, T. et al. (2003) J Neurosci 23, 3353-3363.
Yankner, B. A., Caceres, A., Duffy, L. K. (1990) PNAS 87:9020-9023.
Yoon, S. O., Casaccia-Bonnefil, P., Carter, B., Chao, M. V. (1998) J Neurosci 18, 3273-3281.
Zhang, Y., et al. (2003) J Neurosci 23, 7385-7394.
Zhou, J., Holtzman, D. M., Weiner, R. I., Mobley, W. C. (1994) Proc Natl Acad Sci USA 91, 3824.
Zhou, J., Valletta, J. S., Grimes, M. L., Mobley, W. C. (1995) J Neurochem 65, 1146-1156.

The patents and publications listed herein describe the general skill in the art and are hereby incorporated by reference in their entireties for all purposes and to the same extent as if each was specifically and individually indicated to be incorporated by reference. In the case of any conflict between a cited reference and this specification, the specification shall control. In describing embodiments of the present application, specific terminology is employed for the sake of clarity. However, the invention is not intended to be limited to the specific terminology so selected. Nothing in this specification should be considered as limiting the scope of the present invention. All examples presented are representative and non-limiting. The above-described embodiments may be modified or varied, without departing from the invention, as appreciated by those skilled in the art in light of the above teachings. It is therefore to be understood that, within the scope of the claims and their equivalents, the invention may be practiced otherwise than as specifically described.

1. A compound of Formula I: 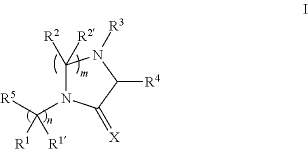
or a pharmaceutically acceptable salt, ester, prodrug or solvate thereof, wherein:
each of R¹, R^{1′}, R², R^{2′}, R³, and R⁴ is independently hydrogen or optionally substituted alkyl; or R² and R^{2′} taken together form ═O, ═S or ═CH₂;
R⁵ is heterocycloalkyl;
X is CH₂, NH, O or S;
n is 0, 1, 2, 3, 4, or 5; and
m is 1 or 2.

2-5. (canceled)

6. A compound of Formula II: 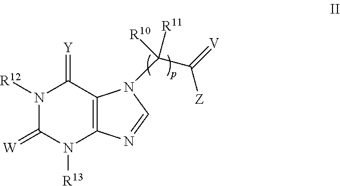
or a pharmaceutically acceptable salt, ester, prodrug or solvate thereof, wherein:
p is 0, 1, 2, 3, 4, 5, or 6;
each of Y, V, and W is independently CH₂, NH, O or S;
each of R¹⁰ and R¹¹ is independently hydrogen or optionally substituted alkyl;
each of R¹² and R¹³ is independently hydrogen, —NR^{a}R^{b}, —OH, —C(═O)OR^{a}, —C(═O)NHR^{a}, —NHC(═O)R^{a}, —NHS(═O)₂R^{a}, or optionally substituted alkyl;
each of R^{a} and R^{b} is independently hydrogen or optionally substituted alkyl; and
Z is an optionally substituted heterocycloalkyl or an optionally substituted heteroaryl.

7-8. (canceled)

9. A compound of Formula III: 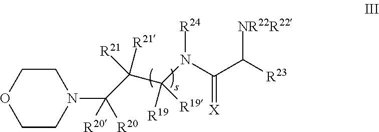
or a pharmaceutically acceptable salt, ester, prodrug or solvate thereof, wherein:
X is CH₂, NH, O or S;
s is 0, 1, 2, 3 or 4;
each of R¹⁹, R^{19′}, R²⁰, R^{20′}, R²¹, R^{21′}, R²², R^{22′} and is independently absent, hydrogen or optionally substituted alkyl; or
R²⁰ and R^{20′} taken together form ═O, ═S, or ═CH₂; or
R²⁰ and R²¹ taken together with the atoms to which they are attached form an optionally substituted cycloalkyl; or
R²⁰ and R²¹ taken together with the atoms to which they are attached form an optionally substituted aryl; or
R¹⁹ and R²⁰ taken together with the atoms to which they are attached form an optionally substituted cycloalkyl; or
R¹⁹ and R²⁰ taken together with the atoms to which they are attached form an optionally substituted aryl; and
R²³ is hydrogen, optionally substituted alkyl, optionally substituted cycloalkyl or optionally substituted aryl; or
R²² and R²³ taken together with the atoms to which they are attached form an optionally substituted heterocycloalkyl;
with the proviso that the compound of Formula III is not 2-amino-3-methyl-N-(2-morpholinoethyl)-butanamide.

10. (canceled)

11. The compound according to claim 9 having a structural formula selected from the group consisting of:

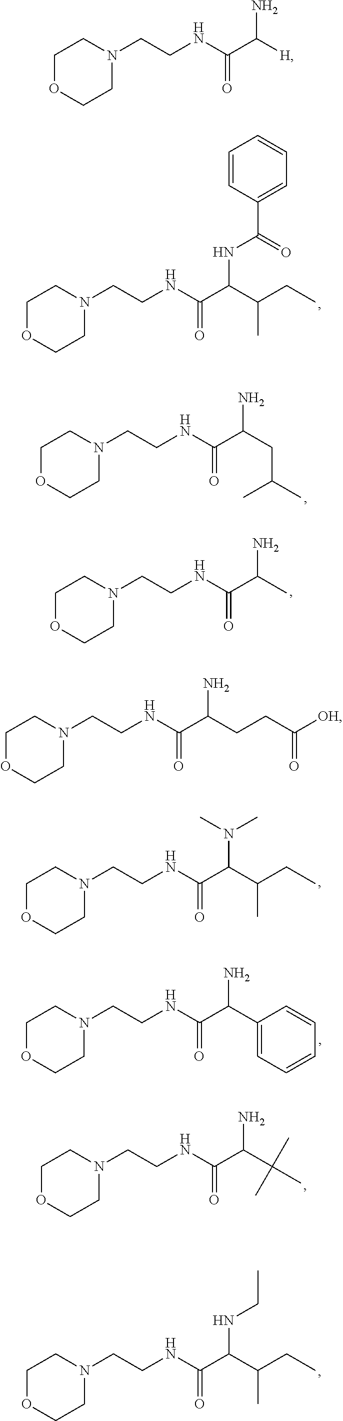

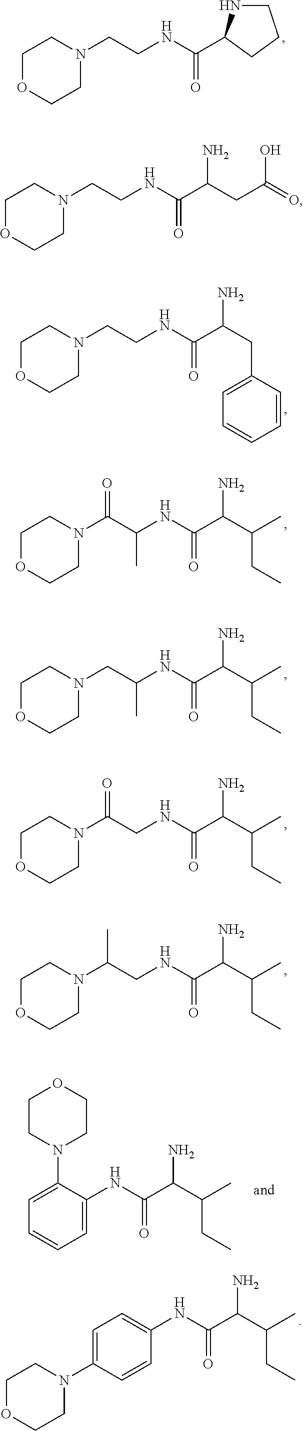

12. A compound of Formula IV: 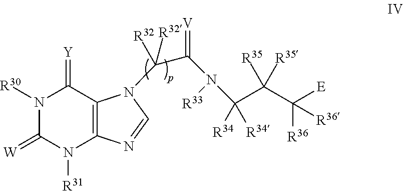
or a pharmaceutically acceptable salt, ester, prodrug or solvate thereof, wherein:
p is 1, 2, 3, 4, 5, or 6;
each of Y, V, and W is independently CH₂, NH, O or S;
each of R³⁰, R³¹, R³², R^{32′} R³³, R³⁴, R^{34′}, R³⁵, R^{35′}, R³⁶, and R^{36′} is independently absent, hydrogen or optionally substituted alkyl; or
R³⁴ and R³⁶ taken together with the atoms to which they are attached form an optionally substituted carbocyclic ring;
E is —CHR^{c}R^{d}, —NR^{c}R^{d}, —OR^{c}, or —SR^{c}; and
each of R^{c} and R^{d} is independently hydrogen or optionally substituted alkyl; or
R^{c} and R^{d} taken together with the nitrogen atom to which they are attached form an optionally substituted heterocyclic ring; or
R^{c} and R^{d} taken together with the carbon atom to which they are attached form an optionally substituted carbocyclic ring;
with the proviso that the compound of Formula IV is not N-(3-(diethylamino)propyl)-2-(4,6-dimethyl-5,7-dioxo-4,5,6,7-tetrahydro-1H-benzo[d]imidazol-1-yl)acetamide.

13-14. (canceled)

15. The compound according to claim 12 having a structural formula selected from the group consisting of:

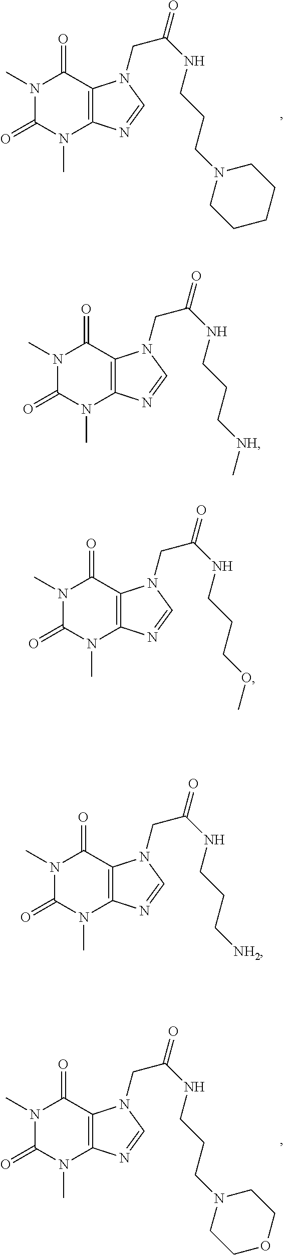

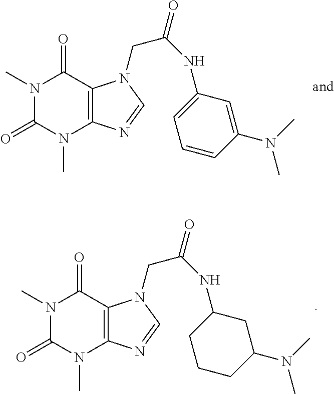

16. A compound selected from the group consisting of
(2R,3R)-2-amino-3-methyl-N-(2-morpholinoethyl)-pentanamide;
(2R,3S)-2-amino-3-methyl-N-(2-morpholinoethyl)-pentanamide; and
(2S,3R)-2-amino-3-methyl-N-(2-morpholinoethyl)-pentanamide;
or a pharmaceutically acceptable salt, solvate, ester, or prodrug thereof.

17. A mixture of two or more compounds selected from the group consisting of
(2S,3S)-2-amino-3-methyl-N-(2-morpholinoethyl)-pentanamide;
(2R,3R)-2-amino-3-methyl-N-(2-morpholinoethyl)-pentanamide;
(2R,3S)-2-amino-3-methyl-N-(2-morpholinoethyl)-pentanamide; and
(2S,3R)-2-amino-3-methyl-N-(2-morpholinoethyl)-pentanamide;
or a pharmaceutically acceptable salt, solvate, ester, or prodrug thereof,
with the proviso that when the mixture consists of (2S,3S)-2-amino-3-methyl-N-(2-morpholinoethyl)-pentanamide and (2R,3R)-2-amino-3-methyl-N-(2-morpholinoethyl)-pentanamide, or a pharmaceutically acceptable salt, solvate, ester, or prodrug thereof; then (2S,3S)-2-amino-3-methyl-N-(2-morpholinoethyl)-pentanamide, or a pharmaceutically acceptable salt, solvate, ester, or prodrug thereof, is in an amount not less than about 5% by weight based on the total amount of the mixture.

18. A mixture of (2R,3R)-2-amino-3-methyl-N-(2-morpholinoethyl)-pentanamide and (2S,3S)-2-amino-3-methyl-N-(2-morpholinoethyl)-pentanamide, or a pharmaceutically acceptable salt, solvate, ester, or prodrug thereof, with the proviso that (2S,3S)-2-amino-3-methyl-N-(2-morpholinoethyl)-pentanamide, or a pharmaceutically acceptable salt, solvate, ester, or prodrug thereof, is in an amount not less than about 5% by weight based on the total amount of the mixture.

19. A mixture of (2R,3S)-2-amino-3-methyl-N-(2-morpholinoethyl)-pentanamide and (2S,3R)-2-amino-3-methyl-N-(2-morpholinoethyl)-pentanamide, or a pharmaceutically acceptable salt, solvate, ester, or prodrug thereof.

20. A pharmaceutical composition comprising the compound of claim 16, or a pharmaceutically acceptable salt, solvate, ester, or prodrug thereof; and a pharmaceutically acceptable carrier.

21. A pharmaceutical composition comprising the mixture of claim 18, or a pharmaceutically acceptable salt, solvate, ester, or prodrug thereof; and a pharmaceutically acceptable carrier.

22. A pharmaceutical composition comprising a pharmaceutically acceptable diluent or carrier and a compound having a structure of one of Formula I, IA, IB, II, IIA, IIB, III, IV or IVA,
wherein Formula (I) has the structure: 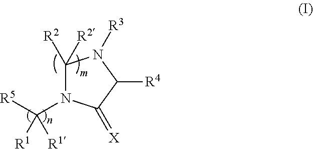
wherein:
each of R¹, R^{1′}, R², R^{2′}, R³, and R⁴ is independently hydrogen or optionally substituted alkyl; or R² and R^{2′} taken together form ═O, ═S or ═CH₂;
R⁵ is heterocycloalkyl;
X is CH₂, NH, O or S;
n is 0, 1, 2, 3, 4, or 5; and
m is 1 or 2;
wherein Formula (IA) has the structure: 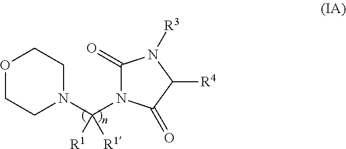
wherein
each of R¹, R^{1′}, R³, and R⁴ independently is hydrogen or optionally substituted alkyl; and
n is 0, 1, 2, 3, 4, or 5;
wherein Formula (IB) has the structure: 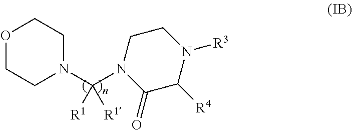
wherein
each of R¹, R^{1′}, R³, and R⁴ is independently hydrogen or optionally substituted alkyl;
wherein Formula (II) has the structure: 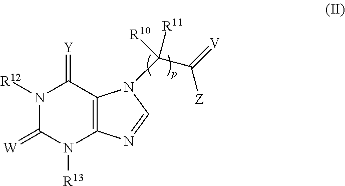
wherein:
p is 0, 1, 2, 3, 4, 5, or 6;
each of Y, V, and W is independently CH₂, NH, O or S;
each of R¹⁰ and R¹¹ is independently hydrogen or optionally substituted alkyl;
each of R¹² and R¹³ is independently hydrogen, —NR^{a}R^{b}, —OH, —C(═O)OR^{a}, —C(═O)NHR^{a}, —NHC(═O)R^{a}, —NHS(═O)₂R^{a}, or optionally substituted alkyl;
each of R^{a} and R^{b} is independently hydrogen or optionally substituted alkyl; and
Z is heterocycloalkyl or heteroaryl wherein each heterocycloalkyl or heteroaryl is bound via a heteroatom and is optionally substituted;
wherein Formula (IIA) has the structure: 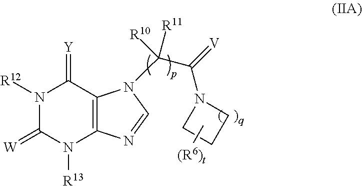
wherein:
p is 0, 1, 2, 3, 4, 5, or 6;
q is 1, 2, 3, or 4;
t is 0, 1, 2, or 3;
each of Y, V, and W is independently O or S; and
each of R⁶ is independently —NR^{a}R^{b}, —OH, —C(═O)OR^{a}, —C(═O)NHR^{a}, —NHC(═O)R^{a}, —NHS(═O)₂R^{a}, or optionally substituted alkyl;
each of R¹⁰ and R¹¹ is independently hydrogen or optionally substituted alkyl;
each of R¹² and R¹³ is independently hydrogen, —NR^{a}R^{b}, —OH, —C(═O)OR^{a}, —C(═O)NHR^{a}, —NHC(═O)R^{a}, —NHS(═O)₂R^{a}, or optionally substituted alkyl;
wherein Formula (IIB) has the structure: 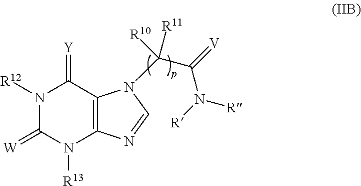
wherein:
p is 0, 1, 2, 3, 4, 5, or 6;
each of Y, V, and W is independently O or S;
each of R¹⁰ and R¹¹ is independently hydrogen or optionally substituted alkyl;
each of R¹² and R¹³ is independently hydrogen, —NR^{a}R^{b}, —OH, —C(═O)OR^{a}, —C(═O)NHR^{a}, —NHC(═O)R^{a}, —NHS(═O)₂R^{a}, or optionally substituted alkyl; and
R′ and R″ taken together with the nitrogen to which they are attached form an optionally substituted heterocyclic aryl;
wherein Formula (III) has the structure: 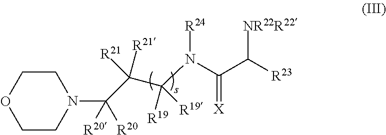
wherein:
X is CH₂, NH, O or S;
s is 0, 1, 2, 3 or 4;
each of R¹⁹, R^{19′}, R²⁰, R^{20′}, R²¹, R^{21′}, R²², R^{22′} and R²⁴ is independently absent, hydrogen or optionally substituted alkyl; or
R²⁰ and R^{20′} taken together form ═O, ═S, or ═CH₂; or
R²⁰ and R²¹ taken together with the atoms to which they are attached form an optionally substituted cycloalkyl; or
R²⁰ and R²¹ taken together with the atoms to which they are attached form an optionally substituted aryl; or
R¹⁹ and R²⁰ taken together with the atoms to which they are attached form an optionally substituted cycloalkyl; or
R¹⁹ and R²⁰ taken together with the atoms to which they are attached form an optionally substituted aryl; and
R²³ is hydrogen, optionally substituted alkyl, optionally substituted cycloalkyl or optionally substituted aryl;
wherein Formula (IV) has the structure: 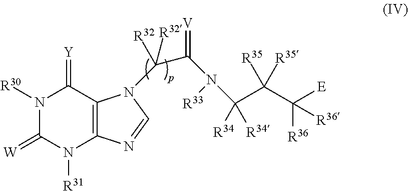
wherein:
p is 1, 2, 3, 4, 5, or 6;
each of Y, V, and W is independently CH₂, NH, O or S;
each of R³⁰, R³¹, R³², R^{32′} R³³, R³⁴, R^{34′}, R³⁵, R^{35′}, R³⁶, and R^{36′} is independently absent, hydrogen or optionally substituted alkyl; or
R³⁴ and R³⁶ taken together with the atoms to which they are attached form an optionally substituted carbocyclic ring;
E is —CHR^{c}R^{d}, —NR^{c}R^{d}, —OR^{c}, or —SR^{c}; and
each of R^{c} and R^{d} is independently hydrogen or optionally substituted alkyl; or
R^{c} and R^{d} taken together with the nitrogen atom to which they are attached form an optionally substituted heterocyclic ring; or
R^{c} and R^{d} taken together with the carbon atom to which they are attached form an optionally substituted carbocyclic ring; and
wherein Formula (IVA) has the structure: 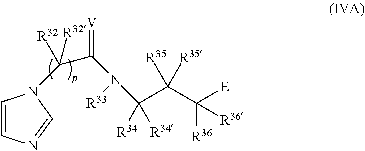
wherein
p is 1, 2, 3, 4, 5, or 6;
V is CH₂, NH, O or S;
each of R³², R^{32′}, R³³, R³⁴, R^{34′}, R³⁵, R^{35′}, R³⁶, and R^{36′} is independently absent, hydrogen or optionally substituted alkyl; or
R³⁴ and R³⁶ taken together with the atoms to which they are attached form an optionally substituted carbocyclic ring;
E is —CHR^{c}R^{d}, —NR^{c}R^{d}, —OR^{c}, and —SR^{c}; and each of R^{c} and R^{d} is independently hydrogen or optionally substituted alkyl; or
R^{c} and R^{d} taken together with the nitrogen atom to which they are attached form an optionally substituted heterocyclic ring; or
R^{c} and R^{d} taken together with the carbon atom to which they are attached form an optionally substituted carbocyclic ring;
or a pharmaceutically acceptable salt, ester, prodrug or solvate thereof.

23. The pharmaceutical composition of claim 22 wherein said compound has the structural formula selected from the group consisting of:

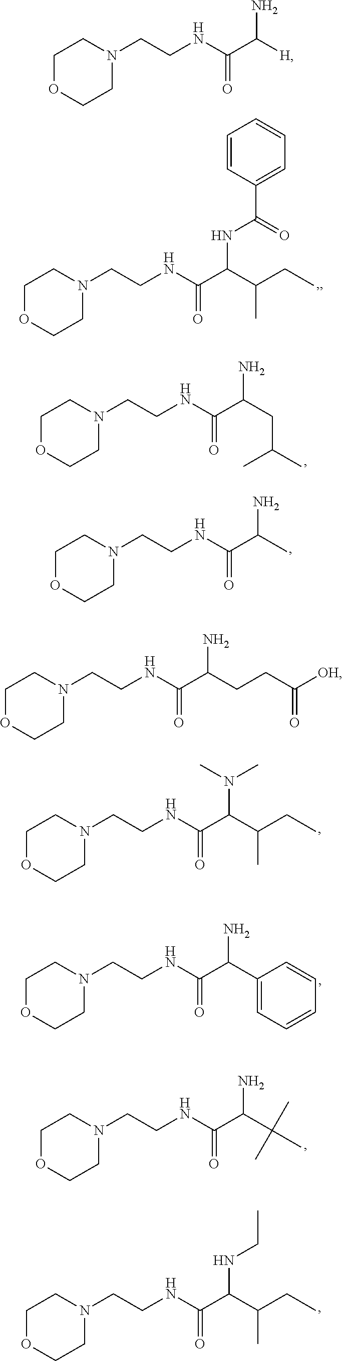

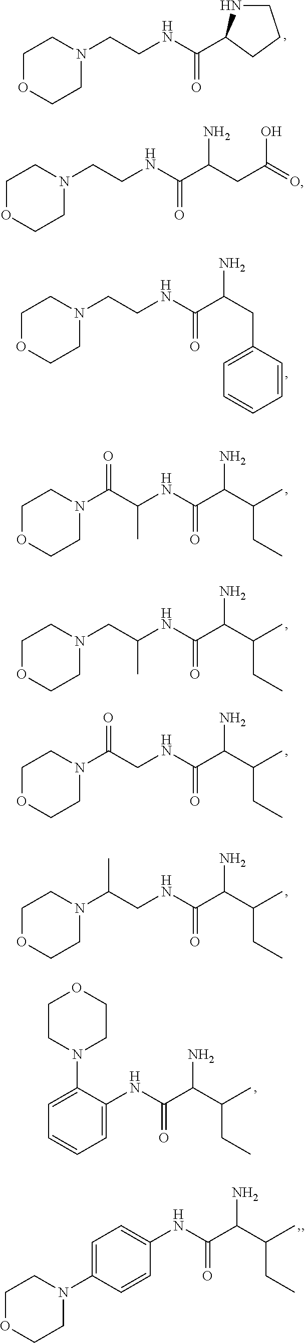

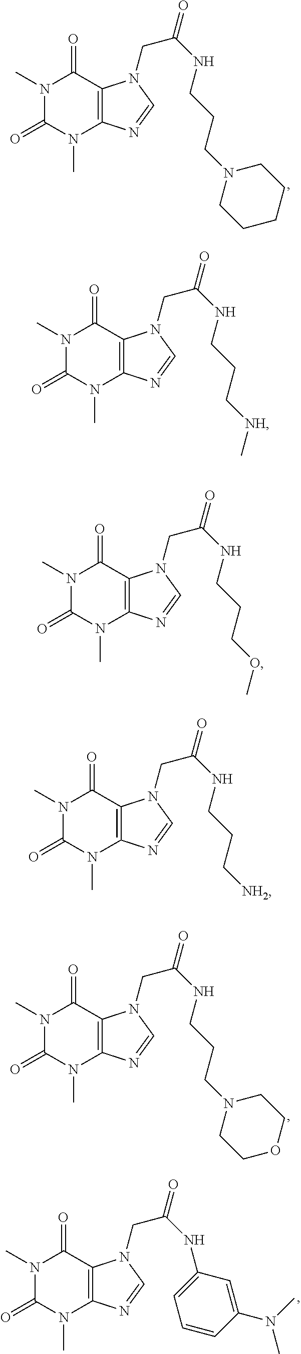

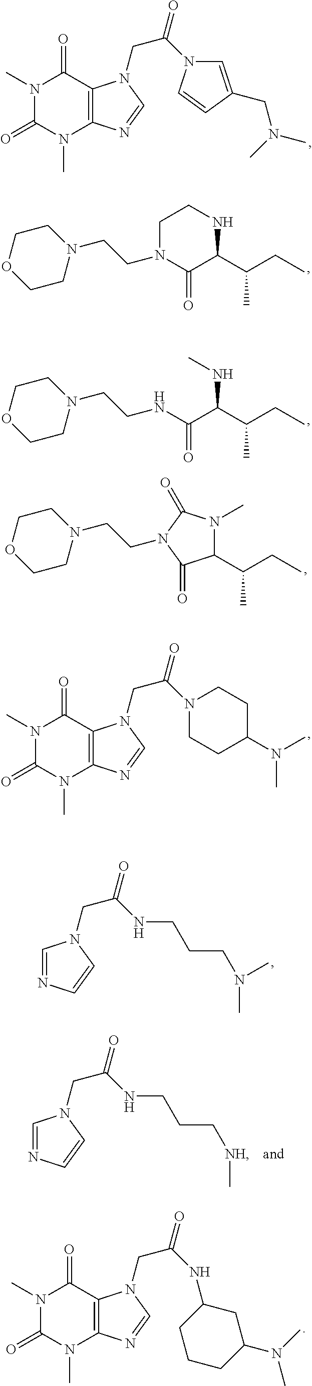

24. A method for treating a disorder associated with p75 expression comprising administering to a patient in need of such treatment a compound having a structure of one of Formula I, IA, IB, II, IIA, IIB, III, IV or IVA, wherein Formula (I) has the structure: 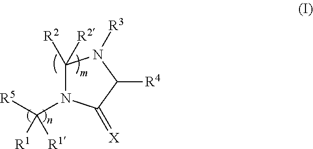
wherein:
each of R¹, R^{1′}, R², R^{2′}, R³, and R⁴ is independently hydrogen or optionally substituted alkyl; or R² and R^{2′} taken together form ═O, ═S or ═CH₂;
R⁵ is heterocycloalkyl;
X is CH₂, NH, O or S;
n is 0, 1, 2, 3, 4, or 5; and
m is 1 or 2;
wherein Formula (IA) has the structure: 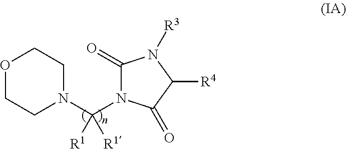
wherein
each of R¹, R^{1′}, R³, and R⁴ independently is hydrogen or optionally substituted alkyl; and
n is 0, 1, 2, 3, 4, or 5;
wherein Formula (IB) has the structure: 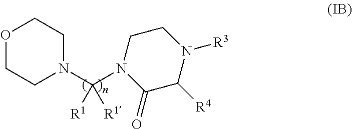
wherein
each of R¹, R^{1′}, R³, and R⁴ is independently hydrogen or optionally substituted alkyl;
wherein Formula (II) has the structure: 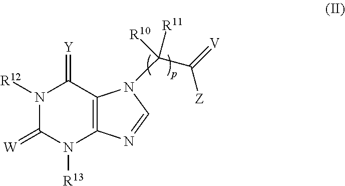
wherein:
p is 0, 1, 2, 3, 4, 5, or 6;
each of Y, V, and W is independently CH₂, NH, O or S;
each of R¹⁰ and R¹¹ is independently hydrogen or optionally substituted alkyl;
each of R¹² and R¹³ is independently hydrogen, —NR^{a}R^{b}, —OH, —C(═O)OR^{a}, —C(═O)NHR^{a}, —NHC(═O)R^{a}, —NHS(═O)₂R^{a}, or optionally substituted alkyl;
each of R^{a} and R^{b} is independently hydrogen or optionally substituted alkyl; and
Z is heterocycloalkyl or heteroaryl wherein each heterocycloalkyl or heteroaryl is bound via a heteroatom and is optionally substituted;
wherein Formula (IIA) has the structure: 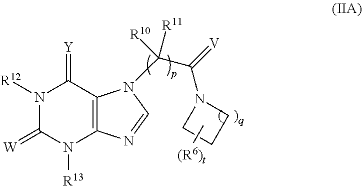
wherein:
p is 0, 1, 2, 3, 4, 5, or 6;
q is 1, 2, 3, or 4;
t is 0, 1, 2, or 3;
each of Y, V, and W is independently O or S; and
each of R⁶ is independently —NR^{a}R^{b}, —OH, —C(═O)OR^{a}, —C(═O)NHR^{a}, —NHC(═O)R^{a}, —NHS(═O)₂R^{a}, or optionally substituted alkyl;
each of R¹⁰ and R¹¹ is independently hydrogen or optionally substituted alkyl;
each of R¹² and R¹³ is independently hydrogen, —NR^{a}R^{b}, —OH, —C(═O)OR^{a}, —C(═O)NHR^{a}, —NHC(═O)R^{a}, —NHS(═O)₂R^{a}, or optionally substituted alkyl;
wherein Formula (IIB) has the structure: 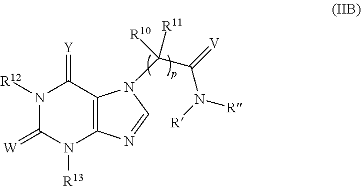
wherein:
p is 0, 1, 2, 3, 4, 5, or 6;
each of Y, V, and W is independently O or S;
each of R¹⁰ and R¹¹ is independently hydrogen or optionally substituted alkyl;
each of R¹² and R¹³ is independently hydrogen, —NR^{a}R^{b}, —OH, —C(═O)OR^{a}, —C(═O)NHR^{a}, —NHC(═O)R^{a}, —NHS(═O)₂R^{a}, or optionally substituted alkyl; and
R′ and R″ taken together with the nitrogen to which they are attached form an optionally substituted heterocyclic aryl;
wherein Formula (III) has the structure: 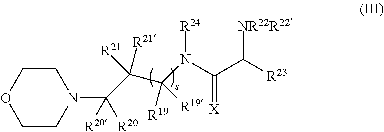
wherein:
X is CH₂, NH, O or S;
s is 0, 1, 2, 3 or 4;
each of R¹⁹, R^{19′}, R²⁰, R^{20′}, R²¹, R^{21′}, R²², R^{22′} and R²⁴ is independently absent, hydrogen or optionally substituted alkyl; or
R²⁰ and R^{20′} taken together form ═O, ═S, or ═CH₂; or
R²⁰ and R²¹ taken together with the atoms to which they are attached form an optionally substituted cycloalkyl; or
R²⁰ and R²¹ taken together with the atoms to which they are attached form an optionally substituted aryl; or
R¹⁹ and R²⁰ taken together with the atoms to which they are attached form an optionally substituted cycloalkyl; or
R¹⁹ and R²⁰ taken together with the atoms to which they are attached form an optionally substituted aryl; and
R²³ is hydrogen, optionally substituted alkyl, optionally substituted cycloalkyl or optionally substituted aryl;
wherein Formula (IV) has the structure: 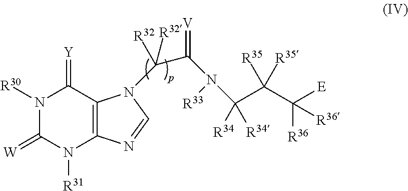
wherein:
p is 1, 2, 3, 4, 5, or 6;
each of Y, V, and W is independently CH₂, NH, O or S;
each of R³⁰, R³¹, R³², R^{32′} R³³, R³⁴, R^{34′}, R³⁵, R^{35′}, R³⁶, and R^{36′} is independently absent, hydrogen or optionally substituted alkyl; or
R³⁴ and R³⁶ taken together with the atoms to which they are attached form an optionally substituted carbocyclic ring;
E is —CHR^{c}R^{d}, —NR^{c}R^{d}, —OR^{c}, or —SR^{c}; and
each of R^{c} and R^{d} is independently hydrogen or optionally substituted alkyl; or
R^{c} and R^{d} taken together with the nitrogen atom to which they are attached form an optionally substituted heterocyclic ring; or
R^{c} and R^{d} taken together with the carbon atom to which they are attached form an optionally substituted carbocyclic ring; and
wherein Formula (IVA) has the structure: 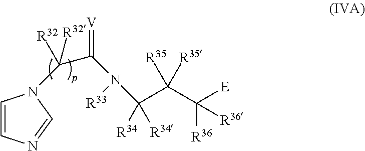
wherein
p is 1, 2, 3, 4, 5, or 6;
V is CH₂, NH, O or S;
each of R³², R^{32′} R³³, R³⁴, R^{34′}, R³⁵, R^{35′}, R³⁶ and R^{36′} is independently absent, hydrogen or optionally substituted alkyl; or
R³⁴ and R³⁶ taken together with the atoms to which they are attached form an optionally substituted carbocyclic ring;
E is —CHR^{c}R^{d}, —NR^{c}R^{d}, —OR^{c}, and —SR^{c}; and each of R^{c} and R^{d} is independently hydrogen or optionally substituted alkyl; or
R^{c} and R^{d} taken together with the nitrogen atom to which they are attached form an optionally substituted heterocyclic ring; or
R^{c} and R^{d} taken together with the carbon atom to which they are attached form an optionally substituted carbocyclic ring;
or a pharmaceutically acceptable salt, ester, prodrug or solvate thereof.

25-27. (canceled)

28. The method according to claim 24 wherein said compound has the structural formula selected from the group consisting of:

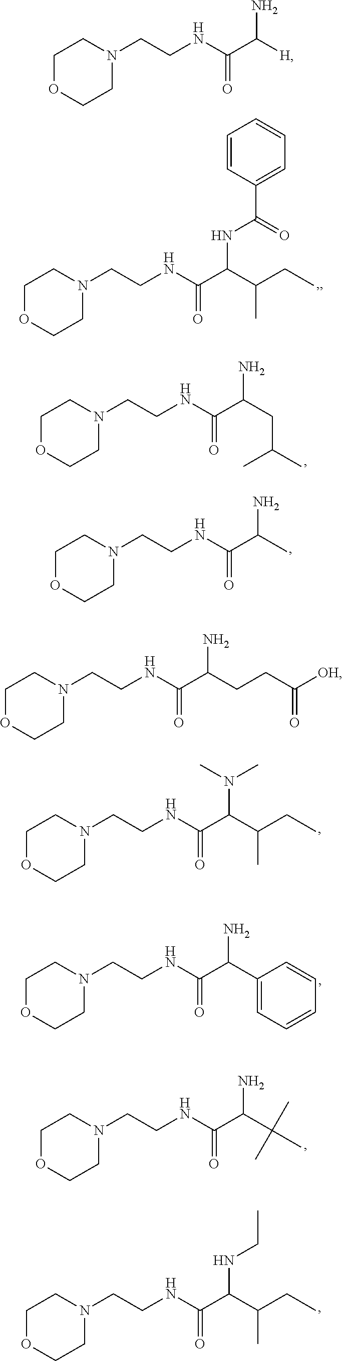

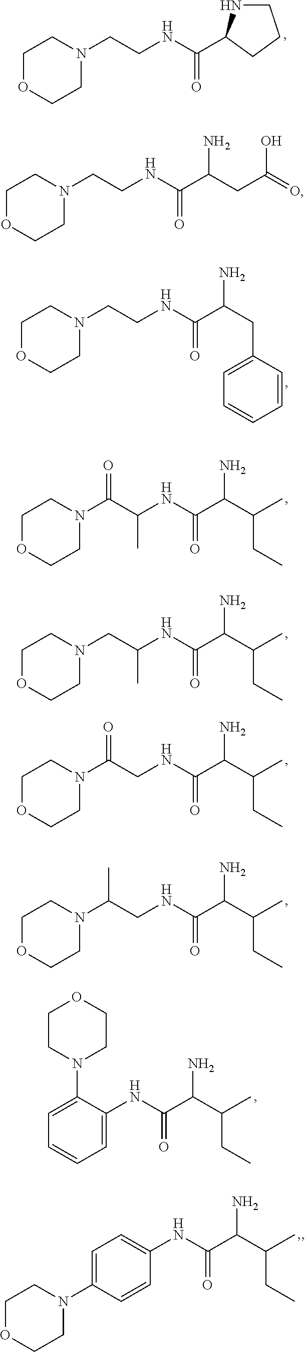

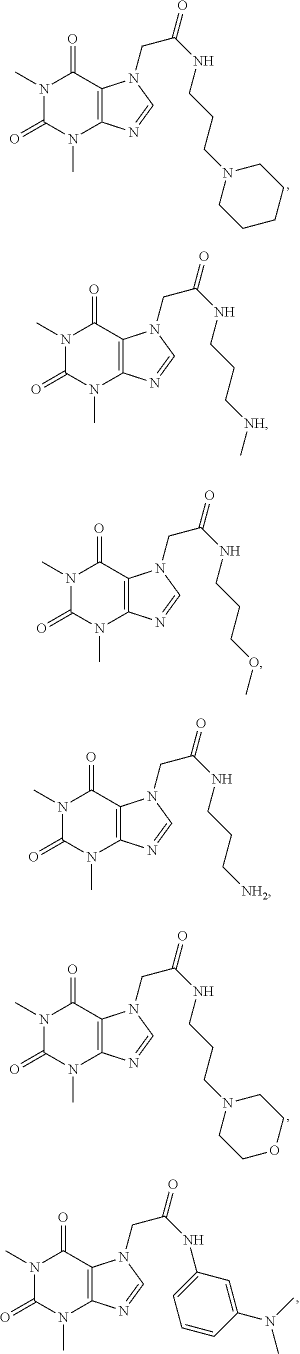

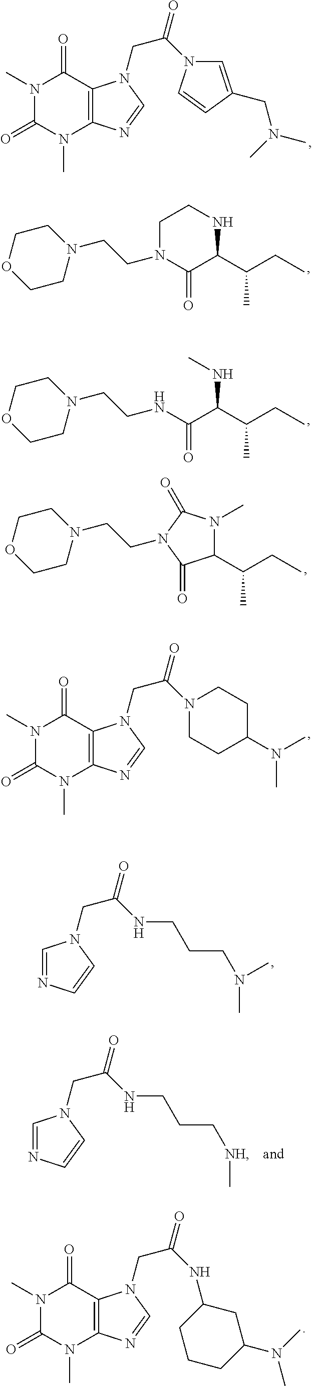

29. A method for treating a disorder associated with p75 expression comprising administering to a patient in need of such treatment a pharmaceutical composition comprising compound selected from the group consisting of
(2R,3R)-2-amino-3-methyl-N-(2-morpholinoethyl)-pentanamide;
(2R,3S)-2-amino-3-methyl-N-(2-morpholinoethyl)-pentanamide; and
(2S,3R)-2-amino-3-methyl-N-(2-morpholinoethyl)-pentanamide;
or a pharmaceutically acceptable salt, solvate, ester, or prodrug thereof.

30. A method for treating a disorder associated with p75 expression comprising administering to a patient in need of such treatment a pharmaceutical composition comprising a mixture of two or more compounds selected from the group consisting of
(2S,3S)-2-amino-3-methyl-N-(2-morpholinoethyl)-pentanamide;
(2R,3R)-2-amino-3-methyl-N-(2-morpholinoethyl)-pentanamide;
(2R,3S)-2-amino-3-methyl-N-(2-morpholinoethyl)-pentanamide; and
(2S,3R)-2-amino-3-methyl-N-(2-morpholinoethyl)-pentanamide;
or a pharmaceutically acceptable salt, solvate, ester, or prodrug thereof,
with the proviso that when the mixture consists of (2S,3S)-2-amino-3-methyl-N-(2-morpholinoethyl)-pentanamide and (2R,3R)-2-amino-3-methyl-N-(2-morpholinoethyl)-pentanamide, or a pharmaceutically acceptable salt, solvate, ester, or prodrug thereof; then (2S,3S)-2-amino-3-methyl-N-(2-morpholinoethyl)-pentanamide, or a pharmaceutically acceptable salt, solvate, ester, or prodrug thereof, is in an amount not less than about 5% by weight based on the total amount of the mixture.

31. A method for treating a disorder associated with p75 expression comprising administering to a patient in need of such treatment a pharmaceutical composition comprising a mixture of (2R,3R)-2-amino-3-methyl-N-(2-morpholinoethyl)-pentanamide and (2S,3S)-2-amino-3-methyl-N-(2-morpholinoethyl)-pentanamide, or a pharmaceutically acceptable salt, solvate, ester, or prodrug thereof, with the proviso that (2S,3S)-2-amino-3-methyl-N-(2-morpholinoethyl)-pentanamide, or a pharmaceutically acceptable salt, solvate, ester, or prodrug thereof, is in an amount not less than about 5% by weight based on the total amount of the mixture.

32. A method for treating a disorder associated with p75 expression comprising administering to a patient in need of such treatment a pharmaceutical composition comprising a mixture of (2R,3S)-2-amino-3-methyl-N-(2-morpholinoethyl)-pentanamide and (2S,3R)-2-amino-3-methyl-N-(2-morpholinoethyl)-pentanamide, or a pharmaceutically acceptable salt, solvate, ester, or prodrug thereof.


SEQUENCE LISTING (3 sequences)

SEQ ID NO: 1 - protein, 5 aa, Homo sapiens
IKGKE

SEQ ID NO: 2 - protein, 5 aa, Rattus sp.
IKGNE

SEQ ID NO: 3 - protein, 5 aa, Homo sapiens
IRGHQ